(19) United States
(12) Patent Application Publication
(10) Pub. No.: US 2025/0228826 A1
(43) Pub. Date: Jul. 17, 2025

(54) COMBINATION OF A GREMLIN-1 ANTAGONIST WITH AN INHIBITOR OF RAS-RAF-MEK-ERK SIGNALLING

(71) Applicant: UCB BIOPHARMA SRL, BRUSSELS (BE)

(72) Inventors: GARETH CHARLES GLYNDWR DAVIES, SLOUGH BERKSHIRE (GB); PAUL JONES, SLOUGH BERKSHIRE (GB); BRAM JEROEN VAN DE SANDE, BRUSSELS (BE); JENNIFER PATRICIA MORTON, GLASGOW (GB)

(21) Appl. No.: 18/854,625

(22) PCT Filed: Apr. 6, 2023
(86) PCT No.: PCT/EP2023/059270

§ 371 Date: Oct. 7, 2024

(30) Foreign Application Priority Data
Apr. 8, 2022 (GB) 2205203.9

(51) Int. Cl. A61K 31/4365; A61K 31/4184; A61K 31/4439; A61K 31/519; A61K 39/395; A61K 45/06; A61P 35/00; C07K 16/18; C12N 15/113; C12Q 1/6886

(52) U.S. Cl. CPC A61K 31/4365; A61K 31/4184; A61K 31/4439; A61K 31/519; A61K 39/3955; A61K 45/06; A61P 35/00; C07K 16/18; C12N 15/113; C12Q 1/6886; C07K 2317/21; C07K 2317/24; C07K 2317/569; C07K 2317/622; C12N 2310/11; C12N 2310/14; C12N 2310/531; C12Q 2600/106; C12Q 2600/158

(57) ABSTRACT

The present invention relates to an anti-GREM1 antagonist for use in a method for the treatment or prevention of a cancer in combination with an inhibitor of Ras-Raf-MEK-ERK signalling.

### FIELD OF THE INVENTION

The present invention relates to combination therapies for the treatment or prevention of a cancer. In particular, the present invention relates to an anti-GREM1 antagonist for use in a method for the treatment or prevention of a cancer in combination with an inhibitor of Ras-Raf-MEK-ERK signalling, and related compositions and kits. The present invention also relates to methods of predicting whether or not a patient is likely to respond to a combination therapy based on stromal GREM1 overexpression and Ras-Raf-MEK-ERK signalling pathway signalling or mutations in a RAS gene or a RAF gene.

### BACKGROUND OF THE INVENTION

Mitogen-activated protein kinase (MAPK) signal transduction pathways (such as the Ras-Raf-MEK-ERK signalling pathway) play a critical role in cell survival, proliferation and differentiation. Dysregulated signalling in these pathways is associated with various diseases, including cancer, due to uncontrolled cell proliferation. Furthermore, upregulated signalling in MAPK pathways has been reported as a resistance mechanism in response to a broad range of anti-cancer therapies, including cytotoxics, immunomodulators and EGFR inhibitors. (Martinelli, E. et al., 2017; Kozar, I. et al., 2019; Kobayashi, Y. et al., 2020). Inhibitors of the MAPK signal transduction pathways, in particular the Ras-Raf-MEK-ERK signal transduction pathway, such as MEK and ERK inhibitors, have been described for treating cancer. For example, MEK inhibitors have been approved for use in treating BRAF-mutated melanoma and KRAS/BRAF mutated colorectal cancer. However, acquired resistance is common in patients receiving treatment with an inhibitor of the MAPK signal transduction pathways, such as the Ras-Raf-MEK-ERK signal transduction pathway. Thus, there is a need to develop new therapies for the treatment and prevention of cancer. Combination therapies that are able to improve the efficacy of existing MAPK pathway inhibitors are also needed.

One cancer that presents a significant challenge is pancreatic cancer. Pancreatic cancer is a fatal disease with increased incidence in recent years. Environmental risk factors associated with pancreatic cancer include smoking, pancreatitis, alcohol, obesity, infection and diet, meaning that pancreatic cancer is becoming increasingly common in developed countries. Surgical resection (pancreatico-duodenectomy) combined with chemotherapy presents the only long-term potential cure for pancreatic cancer. However, limited screening coupled with lack of symptoms means that patients are typically diagnosed with late stage, advanced disease, limiting the chances of surgical resection. As a result, pancreatic cancer has one of the lowest survival rates of all common cancers, with a 5-year survival rate of less than 7% and a poor survival rate of 5% over 10 years or more. Over the last 40 years, there has been limited improvement in survival rate, reinforcing the urgent need for new therapeutic treatments for pancreatic cancer. In non-resectable tumours, the standard of care for pancreatic cancer is chemotherapy regimens. However, chemotherapy is associated with side effects and cancer recurrence is common. Late diagnosis combined with poor prognosis and limited treatment strategies for pancreatic cancer necessitate a need for improved therapies.

### SUMMARY OF THE INVENTION

The inventors have demonstrated that genes involved in Ras-Raf-MEK-ERK signalling, such as NRas, Myc and Mapk1 are upregulated in a LSL-Kras^{G12D/+}; LSL-Trp53^{R172H/+}; Pdx1-Cre (KPC) mouse model upon exposure with an anti-GREM1 antagonist (such as Ab7326). Moreover, the inventors have surprisingly shown that GREM1 antagonists can be administered advantageously in combination with inhibitors of Ras-Raf-MEK-ERK signalling, such as MEK inhibitors, for the treatment or prevention of pancreatic cancer. It is envisaged by the inventors based on these results that combination therapies comprising GREM1 antagonists and inhibitors of Ras-Raf-MEK-ERK signalling, such as MEK inhibitors and ERK inhibitors, will be of general utility in the treatment and prevention of cancers in which Ras-Raf-MEK-ERK signalling is induced/upregulated following treatment with an anti-GREM1 antagonist. Moreover, it is envisaged by the inventors based on these results that combination therapies comprising GREM1 antagonists and inhibitors of Ras-Raf-MEK-ERK signalling, such as MEK and ERK inhibitors, will be of general utility in the treatment and prevention of cancers associated with Ras-Raf-MEK-ERK pathway dysregulation or cancers containing mutations in a Ras or Raf gene. The in vivo results provided herein illustrate significantly increased survival in a LSL-Kras^{G12D/+}; LSL-Trp53^{RI72H/+}; Pdx1-Cre (KPC) mouse model by administration of a GREM1 antagonist in combination with a MEK inhibitor, as well as slower tumour growth and tumour shrinkage. The inventors' findings provide for an improved approach to the prevention and treatment of cancer, in particular, pancreatic cancer.

Thus, in a first aspect of the present invention there is provided an anti-GREM1 antagonist for use in a method for the treatment or prevention of a cancer, wherein the method further comprises administering an inhibitor of Ras-Raf-MEK-ERK signalling.

In a further aspect of the present invention there is provided an inhibitor of Ras-Raf-MEK-ERK signalling for use in a method for the treatment or prevention of cancer wherein the method further comprises administering an anti-GREM1 antagonist.

In another aspect of the present invention there is provided a method of treating a cancer comprising administering a therapeutically effective amount of an anti-GREM1 antagonist in combination with a therapeutically effective amount of an inhibitor of Ras-Raf-MEK-ERK signalling to a subject in need thereof.

In yet another aspect of the present invention there is provided a composition or kit comprising an anti-GREM1 antagonist and an inhibitor of Ras-Raf-MEK-ERK signalling.

In a further aspect of the present invention there is provided a method for determining whether or not a patient having or suspected of having or being at risk of developing cancer is likely to respond to a combination treatment with a GREM1 antagonist and an inhibitor of Ras-Raf-MEK-ERK signalling, which method comprises measuring Ras-Raf-MEK-ERK signalling in the patient in response to treatment with an anti-GREM1 antagonist, and thereby predicting whether or not the patient is likely to respond to treatment with the combination.

In a further aspect of the present invention there is provided a method for determining whether or not a patient having or suspected of having or being at risk of developing cancer is likely to respond to a combination treatment with a GREM1 antagonist and an inhibitor of Ras-Raf-MEK-ERK signalling, which method comprises measuring stromal expression of GREM1, epithelial expression of GREM1 and/or Ras-Raf-MEK-ERK signalling in the patient, and thereby predicting whether or not the patient is likely to respond to treatment with the combination.

In yet another aspect of the present invention there is provided a method for determining whether or not a patient having or suspected of having or being at risk of developing cancer is likely to respond to a combination treatment with a GREM1 antagonist and an inhibitor of Ras-Raf-MEK-ERK signalling, which method comprises measuring stromal expression of GREM1 and/or epithelial expression of GREM1 in the patient and determining whether the patient has a mutation in a RAS gene or a RAF gene, and thereby predicting whether or not the patient is likely to respond to treatment with the combination.

### BRIEF DESCRIPTION OF THE FIGURES

FIG. 1—Kaplan-Meier analysis showing survival of PDAC patients with gremlin-1 expression above or below median as indicated. Patients with tumours expressing high levels of gremlin 1 have a significantly poorer prognosis compared to those with low expression. Generated from KMplotter.

FIG. 2—Schematic showing (a) breeding strategy and (b) experimental design.

FIG. 3—Gene expression in KPC mouse model of 46 genes profiled via NanoString technology. A. Unadjusted p-values depicted in volcano plots. B. Linear model of Nras expression. C. Linear model of Myc expression. D. Linear model of Mapk1 expression.

FIG. 4—Pathway enrichment demonstrating the effect of Ab7326 mIgG1 exposure on KRAS pathways.

FIG. 5—Graph showing tumour burden, as measured by high-resolution ultrasound, in individual mice on treatment (as indicated) in the pharmacodynamic study.

FIG. 6—Graph showing tumour burden, as measured by high-resolution ultrasound, in individual mice on treatment (as indicated) in the survival study cohorts.

FIG. 7—Kaplan-Meier analysis showing survival of Pdx1-Cre; LSL-Kras^{G12D/+}; LSL-Trp53^{R172H/+} (KPC) mice from start of treatment with Ab7326 mIgG1 (n=9), Selumetinib (n=8), or Ab7326 mIgG1 in combination with Selumetinib (n=9), as indicated.

FIG. 8—Graphs of scoring (by HALO software analysis) of IHC using antibodies against markers of cancer associated fibroblasts (aSMA and Podoplanin), and of Picrosirius Red staining for collagen I and III, in mice treated as indicated.

FIG. 9a—Kaplan-Meier analysis showing survival of Pdx1-Cre; LSL-Kras^{G12D/+}; LSL-Trp53^{R172H/+} (KPC) mice from start of treatment with vehicle control (n=10), Ab7326 mIgG1 (n=10), UCB-554 (n=10), or Ab7326 mIgG1 in combination with UCB-554 (n=11), as indicated.

FIG. 9b—Box and whisker plot showing tumour volume on day 7 after start of treatment, as measured by high-resolution ultrasound, in individual KPC mice treated with vehicle control (n=10), Ab7326 mIgG1 (n=8), UCB-554 (n=10), or Ab7326 mIgG1 in combination with UCB-554 (n=11). Each box depicts the interquartile range and the whiskers the min. and max. observations. The solid horizontal bar represents the median and the dashed horizontal line represents the mean volume.

FIG. 9c—Graph showing tumour burden over time, as measured by high-resolution ultrasound, in individual KPC mice on treatment with vehicle control (n=10) or Ab7326 mIgG1 in combination with UCB-554 (n=11).

FIG. 10a—Kaplan-Meier analysis showing survival of Pdx1-Cre; LSL-Kras^{G12D/+}; LSL-Trp53^{R172H/+} (KPC) mice from start of treatment with vehicle control (n=10), Ab7326 mIgG1 (n=19), Selumetinib (n=17), or Ab7326 mIgG1 in combination with Selumetinib (n=19), as indicated. Data are a combined dataset including those shown in FIG. 7.

FIG. 10b—Box and whisker plot showing tumour volume on day 7 after start of treatment, as measured by high-resolution ultrasound, in individual KPC mice treated with vehicle control (n=10), Ab7326 mIgG1 (n=8), Selumetinib (n=9), or Ab7326 mIgG1 in combination with Selumetinib (n=10). Each box depicts the interquartile range and the whiskers the min. and max. observations. The solid horizontal bar represents the median and the dashed horizontal line represents the mean volume.

FIG. 10c—Graph showing tumour burden over time, as measured by high-resolution ultrasound, in individual KPC mice on treatment with vehicle control (n=10) or Ab7326 mIgG1 in combination with Selumetinib (n=10).

### BRIEF DESCRIPTION OF THE TABLES

Table 1—Table showing grem1 upregulated in KPC tumours, and in all pancreatic mouse models combined, compared with Kras^{G12D+} mouse pancreatic duct cells.

Table 2—Table showing dose and schedule of treatments given, number of mice on each treatment, and median survival in each treatment cohort.

Table 3—Table showing dose and schedule of treatments given, number of mice per arm, and timepoint of sampling.

Table 4—Table showing dose and schedule of treatments given, number of mice on each treatment, and median survival in each treatment cohort. * Denotes historical data used.

Table 5—Table comparing survival statistics between the treatment cohorts (Log-Rank analysis). Mice treated with the combination of Ab7326 mIgG1 and Selumetinib show a significant increase in survival compared with Selumetinib alone.

Table 6—Table showing survival data and censoring for individual mice on treatment.

Table 7—Table showing individual mouse samples analysed.

Table 8—Table showing dose and schedule of treatments given, number of mice on each treatment, and median survival in each treatment cohort.

Table 9—Summary of survival statistics for KPC mice treated with Ab7326 and UCB-554 combinations.

Table 10—Summary of survival statistics for KPC mice treated with Ab7326 and Selumetinib combinations.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition, as used in this specification and the appended claims, the singular forms “a”, “an”, and “the” include plural referents unless the content clearly dictates otherwise. Thus, for example, reference to “an inhibitor” includes two or more such inhibitors, or reference to “an oligonucleotide” includes two or more such oligonucleotide and the like.

All publications, patents and patent applications cited herein, whether supra or infra, are hereby incorporated by reference in their entirety.

### Combination Therapy Using an Anti-GREM1 Antagonist and an Inhibitor of Ras-Raf-MEK-ERK Signalling

The present invention provides an anti-GREM1 antagonist in combination with an inhibitor of Ras-Raf-MEK-ERK signalling, such as a MEK or ERK inhibitor, for use in a method for the treatment or prevention of cancer. The cancer is typically a cancer having GREM1 overexpression and/or a cancer associated with Ras-Raf-MEK-ERK signal transduction pathway dysregulation. The cancer may additionally or alternatively be a cancer that displays Ras-Raf-MEK-ERK pathway dysregulation following exposure to/treatment with an anti-GREM1 antagonist. In particular, the cancer may be a cancer in which Ras-Raf-MEK-ERK pathway signalling is induced/upregulated following treatment with an anti-GREM1 antagonist as described herein. Such a cancer may not display Ras-Raf-MEK-ERK pathway dysregulation in the absence of anti-GREM1 antagonist treatment. In other words, the cancer may exhibit normal Ras-Raf-MEK-ERK pathway signalling, relative to a reference sample or reference value, prior to treatment with an anti-GREM1 antagonist as described herein. The cancer may additionally or alternatively be characterised as comprising a mutation in a RAS or RAF gene.

The invention further provides an inhibitor of Ras-Raf-MEK-ERK signalling for use in a method for the treatment or prevention of cancer wherein the method comprises separate, sequential or simultaneous administration of an anti-GREM1 antagonist.

Inhibitor of Ras-Raf-MEK-ERK signalling

An inhibitor of Ras-Raf-MEK-ERK signalling may be any inhibitor that targets the Ras-Raf-MEK-ERK mitogen-activated protein kinase (MAPK) signal transduction cascade (which may also be referred to herein as the classical MAPK pathway, Raf/MEK/ERK, Raf-MEK-ERK, Ras-regulated Raf-MEK-ERK pathway, MAPK/ERK(MEK), Ras/Raf/MAPK(MEK)/ERK), Ras-Raf-MAPK or Raf-MEK-ERK pathway), which are involved in cell survival, proliferation and differentiation. MAPK cascades consist of multiple intracellular signalling pathways. Typically, binding of growth factors to their cognate receptors (such as EGF/EGFR) at the cell surface results in the recruitment of the small GTPase Ras, which activates the serine theronine kinase RAF, resulting in the phosphorylation and activation of mitogen-activated protein kinase kinase (MEK) enzymes, MEK1 and MEK2. MEK1 and MEK2 (also known as Map2k1 and Map2k2, respectively) are dual specificity kinases that phosphorylate Erk1 and Erk2 (MAPK), causing their translocation to the nucleus and activation of multiple transcription factors. Thus, MEK inhibitors usually block MEK1/MEK2 phosphorylation of Erk1/Erk2 and downstream signalling pathways. ERK inhibitors may also prevent phosphorylation of Erk1/Erk2 and/or inhibit the catalytic activity of Erk1/Erk2.

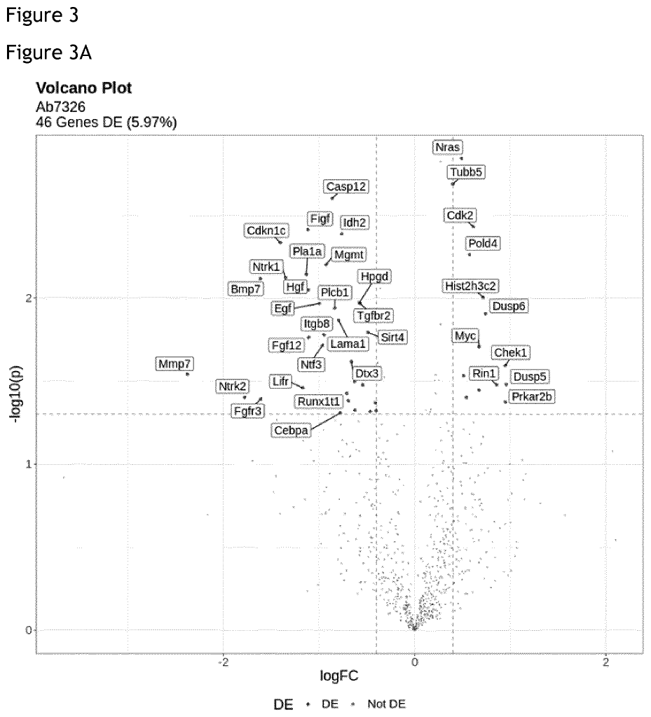

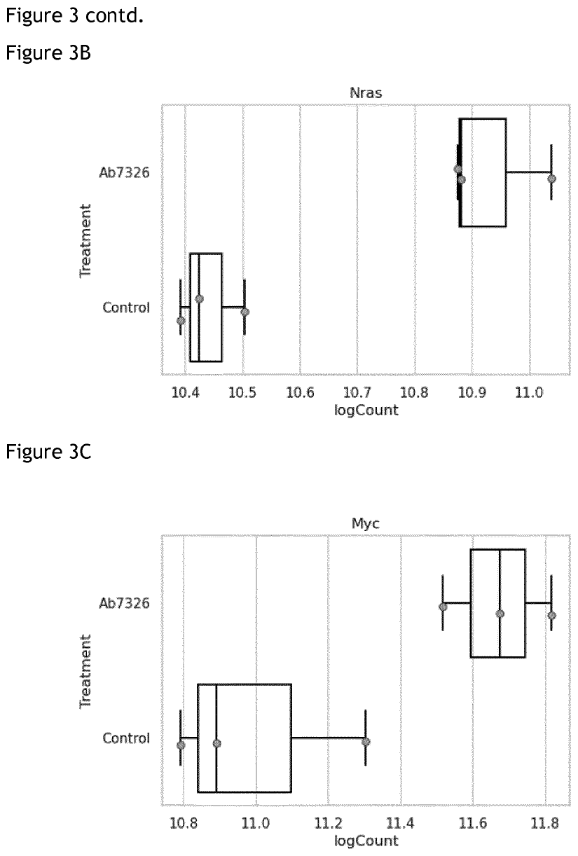

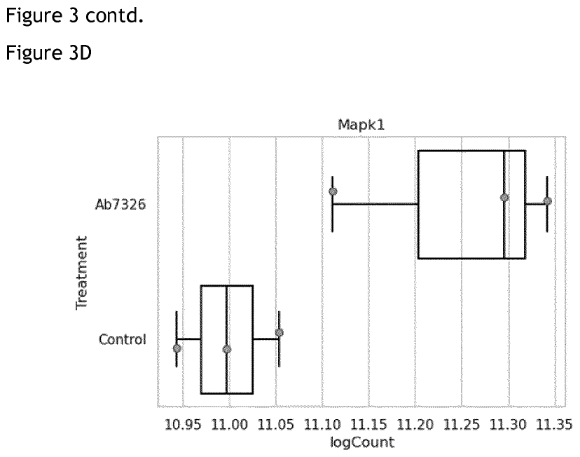

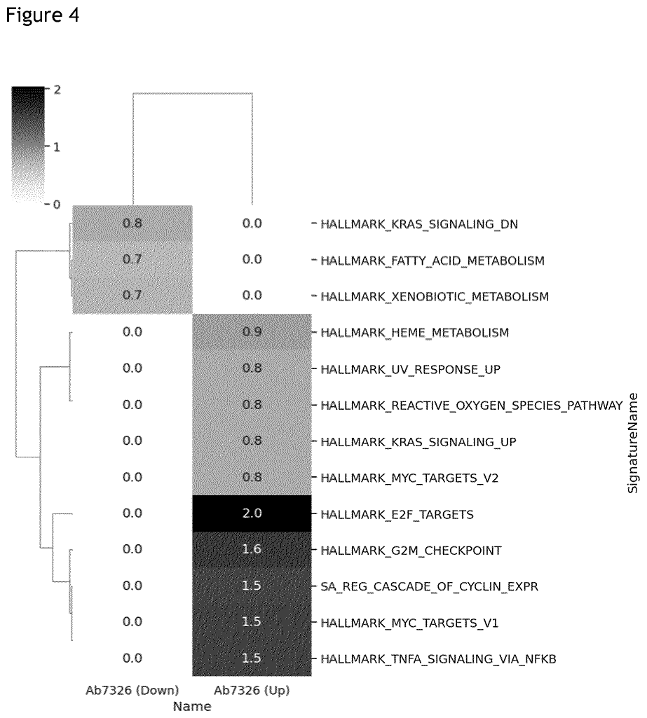

The present inventors have identified that KRAS signalling is upregulated in a KPC mouse model following exposure with an anti-GREM1 antagonist (FIG. 4). Moreover, genes involved in the Ras-Raf-MEK-ERK pathway, such as Mapk1, Nras and Myc are up-regulated upon exposure with the anti-GREM1 antibody Ab7326 in a KPC mouse model (FIG. 3). The present inventors have further shown that the combination of an anti-GREM1 antibody and a MEK inhibitor has significant anti-tumour activity in this model. Thus, a combination therapy comprising an anti-GREM1 antagonist in combination with an inhibitor of Ras-Raf-MEK-ERK signalling may be used for the treatment of cancers associated with GREM1 overexpression, Ras-Raf-MEK-ERK pathway dysregulation and/or mutations in Ras and Raf genes.

In addition, a combination therapy comprising an anti-GREM1 antagonist as defined herein and an inhibitor of Ras-Raf-MEK-ERK signalling, such as a MEK inhibitor or an ERK inhibitor, may be used for the treatment or prevention of cancers in which the Ras-Raf-MEK-ERK pathway is dysregulated following earlier exposure to/treatment with an anti-GREM1 antagonist. In particular, the cancer may be a cancer in which Ras-Raf-MEK-ERK pathway signalling is induced/upregulated following treatment with an anti-GREM1 antagonist as described herein. Such a cancer may not display Ras-Raf-MEK-ERK pathway dysregulation in the absence of anti-GREM1 antagonist treatment. In other words, the cancer may exhibit normal Ras-Raf-MEK-ERK pathway signalling, relative to a reference sample or reference value, prior to treatment with an anti-GREM1 antagonist as described herein.

By dysregulated, it is meant that the Ras-Raf-MEK-ERK pathway is upregulated or downregulated, as compared with a reference sample or reference level. The Ras-Raf-MEK-ERK pathway may be upregulated or downregulated at the mRNA or protein level. For example, the cancer may be a cancer that demonstrates upregulation of genes/proteins in the Ras-Raf-MEK-ERK pathway following exposure to an anti-GREM1 antagonist of the invention. Alternatively or additionally, the cancer may be a cancer that demonstrates increased phosphorylation of Map2kl, Map2k2, Erk1 and/or Erk2 in response to treatment with an anti-GREM1 antagonist of the invention. The cancer may be a cancer or tumour previously described for treatment with a GREM1 antagonist. Such cancers include, but are not limited to, pancreatic cancer, multiple myeloma, colorectal cancer, breast cancer and prostate cancer. In a preferred embodiment, the cancer is pancreatic cancer.

As used herein, an inhibitor of Ras-Raf-MEK-ERK signalling includes pharmaceutically acceptable salts and solvates thereof.

Any protein within the Ras-Raf-MEK-ERK pathway (the classical MAPK cascade) may be targeted by an inhibitor for use in the present invention. Thus, the inhibitor targets at least one component of the Ras-Raf-MEK-ERK pathway to inhibit Ras-Raf-MEK-ERK signalling. Inhibition of Ras-Raf-MEK-ERK signalling typically involves reduction of the activity of at least one component of the pathway. Thus, the inhibitor may be a direct or indirect inhibitor of any node (typically any protein) in the Ras-Raf-MEK-ERK pathway (the classical MAPK cascade). For example, the inhibitor of the Ras-Raf-MEK-ERK pathway may be a direct or indirect RAS inhibitor, such as an inhibitor of KRAS (e.g. ARS-1620 or MRTX849). Alternatively, the inhibitor of Ras-Raf-MEK-ERK signalling may be a RAF inhibitor, such as sorafenib, vemurafenib, dabrafenib or encorafenib. In another aspect, the inhibitor of Ras-Raf-MEK-ERK signalling may be a MEK inhibitor. Exemplary MEK inhibitors are detailed below. In yet a further aspect, the inhibitor of Ras-Raf-MEK-ERK signalling may be an ERK inhibitor, such as ulixertinib (4-[5-chloro-2-[(1-methylethyl)amino]-4-pyridinyl]-N-[(1S)-1-(3-chlorophenyl)-2-hydroxyethyl]-1H-pyrrole-2-carboxamide, monohydrochloride; (S)-4-(5-chloro-2-(isopropylamino)pyridin-4-yl)-N-(1-(3-chlorophenyl)-2-hydroxyethyl)-1H-pyrrole-2-carboxamide; BVD-523; VRT-752271), temuterkib (LY3214996; 4H-Thieno[2,3-c]pyrrol-4-one, 5,6-dihydro-6,6-dimethyl-2-[2-[(1-methyl-1Hpyrazol-5-yl)amino]-4-pyrimidinyl]-5-[2-(4-morpholinyl)ethyl]; 6,6-dimethyl-2-[2-[(2-methylpyrazol-3-yl)amino]pyrimidin-4-yl]-5-(2-morpholin-4-ylethyl)thieno[2,3-c]pyrrol-4-one), JSI-1187, ravoxertinib (1-[(1S)-1-(4-chloro-3-fluorophenyl)-2-hydroxyethyl]-4-[2-[(2-methylpyrazol-3-yl)amino]pyrimidin-4-yl]pyridin-2-one; (S)-1-(1-(4-chloro-3-fluorophenyl)-2-hydroxyethyl)-4-(2-((1-methyl-1H-pyrazol-5-yl)amino)pyrimidin-4-yl)pyridin-2(1H)-one); MK-8353 ((3S)-3-methylsulfanyl-1-[2-[4-[4-(1-methyl-1,2,4-triazol-3-yl)phenyl]-3,6-dihydro-2H-pyridin-1-yl]-2-oxoethyl]-N-[3-(6-propan-2-yloxypyridin-3-yl)-1H-indazol-5-yl]pyrrolidine-3-carboxamide; (S)-N-(3-(6-isopropoxypyridin-3-yl)-1H-indazol-5-yl)-1-(2-(4-(4-(1-methyl-1H-1,2,4-triazol-3-yl)phenyl)-3,6-dihydropyridin-1(2H)-yl)-2-oxoethyl)-3-(methylthio)pyrrolidine-3-carboxamide), or ASN007 ((S)-N-(2-amino-1-(3-chloro-5-fluorophenyl)ethyl)-1-(5-methyl-2-((tetrahydro-2H-pyran-4-yl)amino)pyrimidin-4-yl)-1H-imidazole-4-carboxamide). In a preferred embodiment, the ERK inhibitor is ulixertinib. In an especially preferred embodiment of the invention, the ERK inhibitor ulixertinib and the cancer to be treated is pancreatic cancer, preferably pancreatic ductal adenocarcinoma (PDAC). Other inhibitors of the Ras-Raf-MEK-ERK signalling pathway are well known in the art.

In a preferred aspect of the present invention, the inhibitor of Ras-Raf-MEK-ERK signalling is a MEK inhibitor. A MEK inhibitor for use in combination with the anti-GREM1 antagonist may be an allosteric MEK inhibitor and/or a MEK1 and/or MEK2 inhibitor. The term “allosteric MEK inhibitor” refers to an inhibitor that binds at an allosteric site outside the ATP-binding pocket of MEK. Allosteric MEK inhibitors (otherwise known as type-III MEK inhibitors) demonstrate high selectivity and typically function by reducing movement in the activation loop of MEK1/2. Other MEK inhibitors include Type I and Type II MEK inhibitors, which compete for ATP binding and occupy the catalytic site of MEK (Type I) or bind to adjacent sites within the ATP binding pocket (Type II). All three types of MEK inhibitors are contemplated for use in the methods of the present invention. That is, a Type I, Type II or Type III MEK inhibitor may be used in the methods of the present invention.

Exemplary allosteric MEK inhibitors include selumetinib (AZD6244; ARRY-142886; 6-(4-bromo-2-chloroanilino)-7-fluoro-N-(2-hydroxyethoxy)-3-methylbenzimidazole-5-carboxamide) or trametinib (GSK-1120212; N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl}phenyl)acetamide. Other exemplary MEK inhibitors for use in the present invention include selumetinib (AZD6244), trametinib (GSK-1120212), cobimetinib (GDC-0973; (S)-[3,4-Difluoro-2-(2-fluoro-4-iodophenylamino)phenyl][3-hydroxy-3-(piperidin-2-yl)azetidin-1-yl]methanone), binimetinib (MEK162; 5-((4-bromo-2-fluorophenyl)amino)-4-fluoro-N-(2-hydroxyethoxy)-1-methyl-1H-benzo[d]imidazole-6-carboxamide), CI-1040 (PD184352), Mirdametinib (PD0325901; N-[(2R)-2,3-dihydroxypropoxy]-3,4-difluoro-2-[(2-fluoro-4-iodophenyl)amino]benzamide), TAK733 ((R)-3-(2,3-Dihydroxypropyl)-6-fluoro-5-(2-fluoro-4-iodophenylamino)-8-methylpyrido[2,3-d]pyrimidine-4,7(3H,8H)-dione; 3-[(2R)-2,3-dihydroxypropyl]-6-fluoro-5-(2-fluoro-4-iodoanilino)-8-methylpyrido[2,3-d]pyrimidine-4,7-dione), refametinib (RDEA119/Bay 86-9766; N-[3,4-difluoro-2-(2-fluoro-4-iodoanilino)-6-methoxyphenyl]-1-[(2S)-2,3-dihydroxypropyl]cyclopropane-i-sulfonamide), RO-5126766 (3-[[2-[(Methylaminosulfonyl)amino]-3-fluoropyridin-4-yl]methyl]-4-methyl-7-[(pyrimidin-2-yl)oxy]-2H-1-benzopyran-2-one); N-[3-fluoro-4-[[4-methyl-2-oxo-7-(2-pyrimidinyloxy)-2H-1-benzopyran-3-yl]methyl]-2-pyridinyl]-N′-methyl-sulfamide), RO5068760, Pimasertib (AS703026; N-[(2S)-2,3-dihydroxypropyl]-3-(2-fluoro-4-iodoanilino)pyridine-4-carboxamide), AZD8330 (2-(2-fluoro-4-iodoanilino)-N-(2-hydroxyethoxy)-1,5-dimethyl-6-oxopyridine-3-carboxamide; 2-((2-fluoro-4-iodophenyl)amino)-N-(2-hydroxyethoxy)-15-dimethyl-6-oxo-1,6-dihydropyridine-3-carboxamide), GDC-0623 (5-(2-fluoro-4-iodoanilino)-N-(2-hydroxyethoxy)imidazo[1,5-a]pyridine-6-carboxamide), RO-4987655 (3,4-difluoro-2-(2-fluoro-4-iodoanilino)-N-(2-hydroxyethoxy)-5-[(3-oxooxazinan-2-yl)methyl]benzamide), WX-554 (UCB-554; [3-(Aminomethyl)azetidin-1-yl]-{2-[(2-fluoro-4-iodophenyl)amino]thieno[2,3-b]pyridin-3-yl}-methanone), HL-085, ARRY-300, ClnQ-03, G-573, PD184161 (5-bromo-2-[(2-chloro-4-iodophenyl)amino]-N-(cyclopropylmethoxy)-3,4-difluoro-benzamide), PD318088 (5-bromo-N-(2,3-dihydroxypropoxy)-3,4-difluoro-2-[(2-fluoro-4-iodophenyl)amino]-benzamide), PD98059 (2-(2-Amino-3-methoxyphenyl)-4H-1-benzopyran-4-one), U0126 (1,4-Diamino-2,3-dicyano-1,4-bis(2-aminophenylthio)butadiene; (2Z,3Z)-2,3-Bis[amino[(2-aminophenyl)thio]methylene]butanedinitrile) or SL327.

In a preferred embodiment of the invention, the MEK inhibitor is selumetinib (AZD6244) or trametinib (GSK-1120212). In an especially preferred embodiment of the invention, the MEK inhibitor is selumetinib (AZD6244) or trametinib (GSK-1120212) and the cancer to be treated is pancreatic cancer, preferably pancreatic ductal adenocarcinoma (PDAC).

In a preferred embodiment of the invention, the MEK inhibitor belongs to a class of thieno[2,3-b]pyridine derivatives which are substituted in the 2-position by a substituted anilino moiety. Exemplary MEK inhibitors belonging to this class include:
N-(2-fluoro-4-iodophenyl)-3-(morpholin-4-ylcarbonyl)thieno[2,3-b]pyridin-2-amine;
N-(2-fluoro-4-iodophenyl)-3-[(4-methylpiperazin-1-yl)carbonyl]thieno[2,3-b]pyridin-2-amine;
[2-[((2-fluoro-4-iodophenyl)amino]thieno[2,3-b]pyridin-3-yl](pyrrolidin-1-yl)methanone;
(1-{2-[(2-fluoro-4-iodophenyl)amino]thieno[2,3-b]pyridine-3-carbonyl}piperidin-4-yl)-carbamic acid tert-butyl ester;
2-[((2-fluoro-4-iodophenyl)amino]thieno[2,3-b]pyridine-3-carboxylic acid (4-aminopiperidin-1-yl)amide dihydro-chloride;
(1-{2-[(2-fluoro-4-iodophenyl)amino]thieno[2,3-b]pyridine-3-carbonyl}azetidin-3-ylmethyl)carbamic acid tert-butyl ester;
[3-(aminomethyl)azetidin-1-yl]-{2-[(2-fluoro-4-iodophenyl)amino]thieno[2,3-b]pyridin-3-yl}-methanone;
(1-{2-[(2-fluoro-4-iodophenyl)amino]thieno[2,3-b]pyridine-3-carbonyl}-(3R)-pyrrolidin-3-yl)carbamic acid tert-butyl ester;
(1-{2-[(2-fluoro-4-iodophenyl)amino]thieno[2,3-b]pyridine-3-carbonyl}-(3S)-pyrrolidin-3-yl)carbamic acid tert-butyl ester;
(1-{2-[(2-fluoro-4-iodophenyl)amino]thieno[2,3-b]pyridine-3-carbonyl}azetidin-3-yl)-carbamic acid tert-butyl ester;
[(3R)-3-aminopyrrolidin-1-yl]-{2-[(2-fluoro-4-iodophenyl)amino]thieno[2,3-b]pyridin-3-yl}-methanone;
[(3S)-3-aminopyrrolidin-1-yl]-{2-[(2-fluoro4-iodophenyl)amino]thieno[2,3-b]pyridin-3-yl}-methanone;
(3-aminoazetidin-1-yl)-{2-[(2-fluoro-4-iodophenyl)amino]thieno[2,3-b]pyridin-3-yl]-methanone;
{2-[((2-fluoro-4-iodophenyl)amino]thieno[2,3-b]pyridin-3-yl}-(3-hydroxyazetidin-1-yl)-methanone;
{2-[((2-fluoro-4-iodophenyl)amino]thieno[2,3-b]pyridin-3-yl}-[(3R)-3-hydroxypyrrolidin-1-yl]-methanone;
{2-[((2-fluoro-4-iodophenyl)amino]thieno[2,3-b]pyridin-3-yl}-[(3S)-3-hydroxypyrrolidin-1-yl]-methanone;
{2-[((2-fluoro-4-iodophenyl)amino]thieno[2,3-b]pyridin-3-yl}-[2-(hydroxymethyl)-piperidin-1-yl]-methanone;
{2-[((2-fluoro-4-iodophenyl)amino]thieno[2,3-b]pyridin-3-yl}-[(3S)-3-(hydroxymethyl)-morpholin-4-yl]-methanone;
4-{2-[(2-fluoro-4-iodophenyl)amino]thieno[2,3-b]pyridine-3-carbonyl}-(3R)-3-(hydroxymethyl)piperazine-1-carboxylic acid tert-butyl ester;
2-[4({2-[(2-fluoro-4-iodophenyl)amino]thieno[2,3-b]pyridin-3-yl}carbonyl)piperazin-1-yl]ethanol;
3-((1,4-diazepan-1-ylcarbonyl)-N-(2-fluoro-4-iodophenyl)thieno[2,3-b]pyridin-2-amine;
N-(2-fluoro-4-iodophenyl)-3-(piperazin-1-ylcarbonyl)thieno[2,3-b]pyridin-2-amine;
ethyl [4-({2-[(2-fluoro-4-iodophenyl)amino]thieno[2,3-b]pyridin-3-yl}carbonyl)-piperazin-1-yl]acetate;
[4-({2-[(2-fluoro-4-iodophenyl)amino]thieno[2,3-b]pyridin-3-yl}carbonyl)piperazin-1-yl]acetic acid;
{2-[((2-fluoro-4-iodophenyl)amino]thieno[2,3-b]pyridin-3-yl}-[(2R)-2-(methoxymethyl)-pyrrolidin-1-yl]-methone; and
{2-[((2-fluoro-4-iodophenyl)amino]thieno[2,3-b]pyridin-3-yl}-[(2R)-2-(hydroxymethyl)-piperazin-1-yl]-methanone.

Methods of making these MEK inhibitors are disclosed in WO 2007/088345, which is incorporated herein by reference in its entirety.

Preferred MEK inhibitors belonging to the class of thieno[2,3-b]pyridine derivatives which are substituted in the 2-position by a substituted anilino moiety for use in the present invention are represented by the compounds of formula (II), and pharmaceutically acceptable salts, solvates and N-oxides thereof:

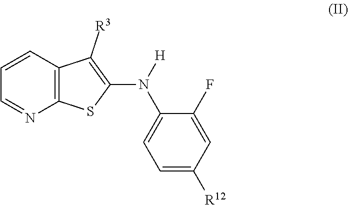

wherein
R¹² represents halogen;
R³ represents CONR^{b}R^{c}; and
R^{b} and R^{c}, when taken together with the nitrogen atom to which they are both attached, represent azetidin-1-yl, pyrrolidin-1-yl, piperidin-1-yl, morpholin-4-yl, thiomorpholin-4-yl, piperazin-1-yl, homopiperidin-1-yl, homo-morpholin-4-yl or homopiperazin-1-yl, any of which groups may be optionally substituted by one or more substituents selected from C₁₋₆ alkyl, C₁₋₆ alkoxy, hydroxy, hydroxy(C₁₋₆)akyl, amino-(C₁₋₆)alkyl, (amino)(hydroxy)(C₁₋₆)alkyl, halogen, oxo, C₂₋₆ akylcarbonyl, carboxy, C₂₋₆ alkoxycarbonyl, di(C₁₋₆)alhylhydrazinylcarbonyl, amino, C₁₋₆ alkylamino, di(C₁₋₆)alkyl-amino, C₂₋₆ alkylcarbonylamino, aminocarbonylamino, aminocarbonyl, C₁₋₆ alkylaminocarbonyl, di(C₁₋₆)alkylaminocarbonyl, aminosulfonyl, C₁₋₆ alkylsulfonyl, C₁₋₆ alkylaminocarbonyl (C₁₋₆)alkyl, C₁₋₆ alkoxy(C₁₋₆)alkyl, carboxy(C₁₋₆)alkyl, C₂₋₆ alkoxycarbonyl(C₁₋₆)alkyl, C₂₋₆ alkoxycarbonylamino and C₂₋₆ alkoxycarbonylamino-(C₁₋₆)alkyl.
   In one embodiment, R¹² is bromo. In another embodiment, R¹² is iodo.
   Methods of making compounds of formula (II) are disclosed in WO 2007/088345, which is incorporated herein by reference in its entirety.

In a preferred embodiment of the invention, the MEK inhibitor is a compound of formula (II), or pharmaceutically acceptable salt, solvate or N-oxide thereof. In an especially preferred embodiment of the invention, the MEK inhibitor is a compound of formula (II), or a pharmaceutically acceptable salt, solvate or N-oxide thereof and the cancer to be treated is pancreatic cancer, preferably pancreatic ductal adenocarcinoma (PDAC).

In a preferred embodiment of the invention, the MEK inhibitor is WX-554 (UCB-554). In an especially preferred embodiment of the invention, the MEK inhibitor is WX-554 (UCB-554) and the cancer to be treated is pancreatic cancer, preferably pancreatic ductal adenocarcinoma (PDAC).

The structure of WX-554 (UCB-554); [3-(Aminomethyl)azetidin-1-yl]-{2-[(2-fluoro-4-iodophenyl)amino]thieno[2,3-b]pyridin-3-yl}-methanone) is depicted here:

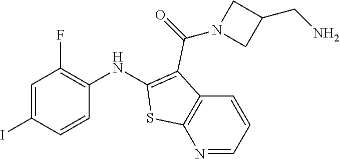 [SMILES: NCC1CN(C(=O)c2c(Nc3ccc(I)cc3F)sc3ncccc23)C1]

Methods of making WX-554 are disclosed in WO 2007/088345, which is incorporated herein by reference in its entirety.

The above described MEK inhibitors belonging to a class of thieno[2,3-b]pyridine derivatives which are substituted in the 2-position by a substituted anilino moiety are potent and selective MEK inhibitors having a binding affinity (IC₅₀) for the human MEK1 and/or MEK2 enzyme of 50 M or less, generally of 20 M or less, usually of 5 M or less, typically of 1 M or less, suitably of 500 nM or less, ideally of 100 nM or less, and preferably of 20 nM or less (the skilled person will appreciate that a lower IC₅₀ figure denotes a more active compound). These MEK inhibitors for use in the present invention may possess at least a 10-fold selective affinity, typically at least a 20-fold selective affinity, suitably at least a 50-fold selective affinity, and ideally at least a 100-fold selective affinity, for the human MEK1 and/or MEK2 enzyme relative to other human kinases.

A variety of methods can be used to determine the activity of a MEK inhibitor, such as an in vitro MEK assay. An exemplary in vitro MEK assay is described in WO 2007/088345, which is incorporated herein by reference in its entirety.

It is also contemplated that a combination therapy comprising a GREM1 antagonist as defined herein and an inhibitor of Ras-Raf-MEK-ERK signalling, such as a MEK inhibitor or an ERK inhibitor, can be used for the treatment or prevention of cancers that are typically targeted with an inhibitor of Ras-Raf-MEK-ERK signalling. The cancer may be a cancer or tumour previously described for treatment with an inhibitor of Ras-Raf-MEK-ERK signalling. Such cancers include, but are not limited to, pancreatic cancer, multiple myeloma, colorectal cancer, lung cancer and melanoma.

The cancer may be a cancer that is responsive to treatment with an inhibitor of Ras-Raf-MEK-ERK signalling and/or a cancer that is contemplated for treatment with an inhibitor of Ras-Raf-MEK-ERK signalling. Alternatively, the cancer may be a cancer that is poorly responsive, non-responsive or refractory to treatment with an inhibitor of Ras-Raf-MEK-ERK signalling. The cancer may be previously described as being unsuitable for treatment with an inhibitor of Ras-Raf-MEK-ERK signalling. In some instances, the cancer may be initially responsive to treatment with an inhibitor of Ras-Raf-MEK-ERK signalling, but develop resistance to the inhibitor of Ras-Raf-MEK-ERK signalling.

Responsiveness may be measured by any means. For example, responsiveness to treatment may be assessed by measuring tumour size before and after treatment using X-ray, CT or MRI scans or by measuring tumour markers. Blood tests to determine organ function may also be used to assess responsiveness to treatment. One exemplary blood marker for monitoring responsiveness in pancreatic cancer is CA19-9. A person skilled in the art knows how to measure responsiveness of a cancer to treatment.

### Cancer

In the context of a combination therapy comprising an anti-GREM1 antagonist and an inhibitor of Ras-Raf-MEK-ERK signalling, such as a MEK or ERK inhibitor, the cancer may be any cancer or tumour. In particular, in the context of a combination therapy comprising an anti-GREM1 antagonist and an inhibitor of Ras-Raf-MEK-ERK signalling, the cancer may be any cancer or tumour having a stroma, typically a desmoplastic stroma. The cancer may be any cancer or tumour which is GREM1-initiated. The cancer may be any cancer where stromal and/or epithelial GREM1 overexpression is observed. The cancer or tumour may have stromal GREM1 overexpression and no epithelial GREM1 overexpression. The cancer or tumour may have epithelial GREM1 overexpression and no stromal GREM1 overexpression. In preferred embodiments, the cancer or tumour has an overexpression of GREM1 in the desmoplastic stroma. The cancer or tumour may be any cancer or tumour suitable for targeting with a GREM1 antagonist, a MEK inhibitor and/or an ERK inhibitor, including any cancer or tumour known in the art to be suitable for targeting with such agent(s), and any cancer or tumour known in the art to be suitable for treatment with such agent(s).

The cancer or tumour may be a solid tumour. The solid tumour may have a desmoplastic stroma.

Anti-GREM1 antagonists have been previously shown to be effective for treatment of a range of cancers in WO 2019/243801 filed on 18 Jun. 2019, the disclosure of which is incorporated herein by reference in its entirety.

Particularly preferred cancers that may be treated include colorectal cancer, multiple myeloma, pancreatic cancer, bladder cancer, breast cancer, lung cancer, stomach cancer, duodenal cancer, oesophageal cancer, head and neck cancer, prostate cancer, glioma, endometrial cancer, ovarian cancer, liver cancer, spleen cancer, bone-resident cancer, melanoma and osteosarcoma. The cancer that may be treated may be intestinal cancer, colon cancer, or rectal cancer. The cancer to be treated may be a disseminated cancer, for example a metastatic cancer. A disseminated cancer should be understood as one that has spread from its initial site of origin within the body. For example, a disseminated cancer could be one that originated in the pancreas, bone marrow, colon, prostate, or breast tissue of a patient but has spread to e.g. the patient's liver or lung.

The combination therapy comprising a GREM1 antagonist and an inhibitor of Ras-Raf-MEK-ERK signalling may also be used to prevent the dissemination of a cancer.

The combination therapy comprising a GREM 1 antagonist and an inhibitor of Ras-Raf-MEK-ERK signalling may be used to prevent polyposis associated with a cancer.

Grading systems are used in cancer biology and medicine to categorize cancer cells with respect to their lack of cellular differentiation. This reflects the extent to which the cancer cells differ in morphology from healthy cells found in the tissue from which the cancer cell originated. The grading system can be used to provide an indication of how quickly a particular cancer might be expected to grow. Typically used grades of cancer are Grades (G) X and 1 to 4. GX indicates that the cancer grade cannot be assessed. G1 (low grade) cancer cells have a similar morphology to normal, healthy, cells (i.e. they are well differentiated) and would be expected to grow slowly, and are less likely to spread. G2 (intermediate grade) cancer cells are moderately differentiated, i.e. they appear more abnormal and would be expected to grow slightly faster than G1 cells. G3 (high grade) cancer cells have a very different morphology compared to normal cells (i.e. they are poorly differentiated) and would be expected to grow faster than G1 and G2 cells. G4 (high grade) cancer cells are undifferentiated (also referred to as anaplastic) and would be expected to have the highest capacity for proliferation.

Cancer grading is different to cancer staging, which gives an indication of how a cancer might spread. A common cancer staging system has five stages, namely Stage 0: cancer cells in situ (i.e. located in their normal tissue); Stage I: cancers are localized to one part of the body; Stage II: cancers are locally advanced; Stage III: cancers are also locally advanced (whether a cancer is designated as Stage II or Stage III can depend on the specific type of cancer); and Stage IV: cancers have often metastasized, or spread to other organs or throughout the body.

A person skilled in the art knows how to determine the grade and/or stage of a cancer. In one embodiment, the invention relates to use of an anti-GREM1 antagonist in combination with an inhibitor of Ras-Raf-MEK-ERK signalling for the treatment and/or prevention of an established cancer. In one embodiment, the cancer is an established cancer. An established cancer may be a high grade cancer, for example a G3 or a G4 cancer. An established cancer may be a cancer that is Stage II or above. An established cancer may be a Stage III or a stage IV cancer. In one embodiment, the established cancer is a Stage IV cancer that has metastasized. In one embodiment, the established cancer is an established pancreatic cancer.

In addition to specifically exemplified applications in treatment and prevention of pancreatic cancer, the inventors envisage that the therapeutic efficacy of GREM1 antagonists in combination with an inhibitor of Ras-Raf-MEK-ERK signalling as illustrated in the Examples will also be applicable to treatment of other cancers having corresponding features as described herein.

In particular, it is envisaged that combinations comprising GREM1 antagonists will be useful in preventing or treating cancers in which the Ras-Raf-MEK-ERK pathway is dysregulated in response to treatment with/exposure to an anti-GREM1 antagonist as described herein. In particular, the cancer may be a cancer in which Ras-Raf-MEK-ERK signalling is induced/upregulated following treatment with an anti-GREM1 antagonist as described herein. Such a cancer may not display Ras-Raf-MEK-ERK pathway dysregulation in the absence of anti-GREM1 antagonist treatment. In other words, the cancer may exhibit normal Ras-Raf-MEK-ERK signalling, relative to a reference sample or reference value, prior to treatment with an anti-GREM1 antagonist as described herein.

It is also envisaged that combinations comprising GREM1 antagonists will be useful in preventing or treating cancers in which there is stromal and/or epithelial GREM1 overexpression, and this overexpression contributes to malignant cell growth. Such cancers include pancreatic cancer, bladder cancer, lung cancer, stomach cancer, duodenal cancer, oesophageal cancer, head and neck cancer, glioma, endometrial cancer, liver cancer, spleen cancer, bone-resident cancer, and osteosarcoma.

Antagonists of the present invention are used in treating or preventing cancer in combination with an inhibitor of Ras-Raf-MEK-ERK signalling (such as a MEK inhibitor or an ERK inhibitor). Prevention of cancer may include preventing the subject from ever being diagnosed with cancer or deferring the onset of cancer. Prevention of cancer may also include prevention of relapse or recurrence of cancer in a subject who has been previously diagnosed with cancer. Prevention of cancer may additionally include increasing the survival of a subject who has not been diagnosed with cancer or who has been previously diagnosed with cancer.

Treatment of cancer may ameliorate one or more symptoms of, induce or prolong remission from, or delay relapse or recurrence of the cancer. Treatment may cure, alleviate or partially arrest the cancer. It may result in a decrease in severity of disease symptoms, or an increase in frequency or duration of symptom-free periods. Treatment of cancer may also include preventing a cancer (e.g. an established cancer) from spreading from its initial site within a patient's body to one or more secondary sites within the patient's body. Thus, treatment of cancer may include prevention of the dissemination or the metastasis of an existing cancer. In a preferred embodiment, antagonists of the present invention are used in treating or preventing pancreatic cancer in combination with an inhibitor of Ras-Raf-MEK-ERK signalling (such as a MEK inhibitor or ERK inhibitor). Treatment of pancreatic cancer may result in a reduction in primary tumour size, for example as assayed by CT or endoscopic ultrasound. Such a reduction may facilitate Whipple procedure (pancreaticoduodenectomy) for the removal of tumours from the head of the pancreas. The inventors' have demonstrated that treatment with an anti-GREM1 antagonist in combination with a MEK inhibitor can increase survival in a KPC mouse model. Thus, treatment of pancreatic cancer with an anti-GREM1 antagonist and a MEK inhibitor may also improve patient survival. It is envisaged that treatment of cancer, particularly pancreatic cancer, with an ERK inhibitor would elicit similar advantageous results.

### Pancreatic Cancer

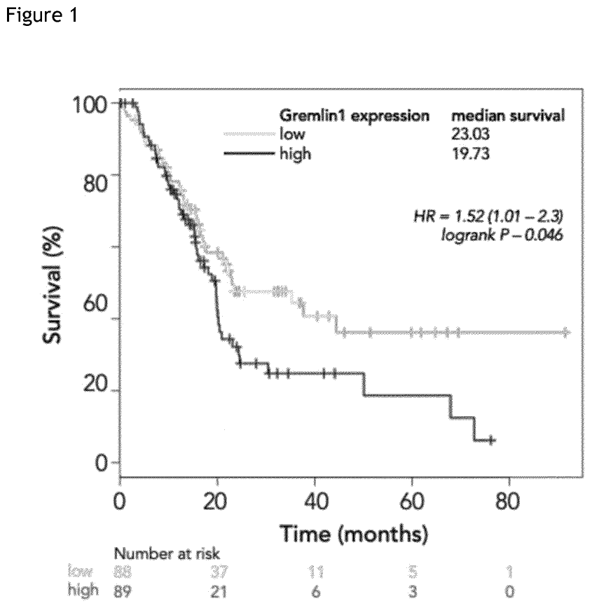

The invention is preferably directed to the treatment or prevention of pancreatic cancer. As described in more detail in the Examples, the present inventors have confirmed GREM1 overexpression in human pancreatic ductal adenocarcinoma samples, and demonstrated that high expression of Grem1 mRNA is significantly associated with poor prognosis (FIG. 1). Moreover, the present inventors have demonstrated that KRAS signalling is upregulated in a KPC mouse model following exposure with an anti-GREM1 antagonist (FIG. 4) and that genes involved in the Ras-Raf-MEK-ERK signalling pathway, such as Mapk1, Nras and Myc are up-regulated upon exposure with the anti-GREM1 antibody Ab7326 in a KPC mouse model (FIG. 3). The present inventors have also shown that a combination therapy comprising an anti-GREM1 antagonist and a MEK inhibitor significantly increased survival, slowed tumour growth and promoted tumour shrinkage in a KPC mouse model. KPC mice develop tumours that are similar to human pancreatic tumours in terms of histology and pathology and mimic human pancreatic cancer.

Accordingly, in one embodiment, the pancreatic cancer is a pancreatic cancer that is characterised by having overexpression of GREM1. In another embodiment, the pancreatic cancer may be characterised as having exocrine tumours or neuroendocrine tumours. Pancreatic neuroendocrine cancers (also known as islet cell tumours) develop in the endocrine gland of the pancreas. An especially preferred form of pancreatic cancer is an exocrine pancreatic cancer, such as pancreatic ductal adenocarcinoma (PDAC). PDAC has been described to account for 90% of all pancreatic carcinomas (Feldmann et al. J Hepatobiliary Pancreat Surg. 2007; 14(3): 224-32). The pancreatic cancer may be any cancer which develops in the lining in the ducts of the pancreas.

The Ras-Raf-MEK-ERK signalling pathway has been reported to have an important role in pancreatic ductal adenocarcinomas (Collisson E A. et al. (2012) A central role for RAF→MEK→ERK signaling in the genesis of pancreatic ductal adenocarcinoma. Cancer Discov. 2012 2(8):685-93). For example, activating mutations in the KRAS oncogene are known to drive PDAC formation by promoting enhanced cellular proliferation, motility and survival, yet targeting KRAS signalling remains challenging and clinical trials using MEK inhibitors have largely failed to achieve statistically significant results in patients (Brauswetter et al. (2017) Molecular subtype specific efficacy of MEK inhibitors in pancreatic cancers. PLoS ONE 12(9): e0185687). Thus, in one embodiment, the pancreatic cancer for treatment is a pancreatic cancer having Ras-Raf-MEK-ERK pathway dysregulation and/or mutations in a Ras (such as KRAS, NRAS and/or HRAS) or Raf gene (such as ARAF, BRAF and/or CRAF), as described herein. For example, the pancreatic cancer may be a pancreatic cancer tumour having a KRAS^{G12D} or KRAS^{G12R} mutation/substitution. Alternatively, the pancreatic cancer may be a pancreatic cancer tumour having a BRAF^{V600E} mutation/substitution.

In another embodiment, the pancreatic cancer is a pancreatic cancer in which the Ras-Raf-MEK-ERK pathway is dysregulated in response to treatment with/exposure to an anti-GREM1 antagonist as described herein. In particular, the pancreatic cancer may be a pancreatic cancer in which Ras-Raf-MEK-ERK signalling is induced/upregulated following treatment with an anti-GREM1 antagonist. Such a pancreatic cancer may not display Ras-Raf-MEK-ERK pathway dysregulation in the absence of anti-GREM1 antagonist treatment. In other words, the pancreatic cancer may exhibit normal Ras-Raf-MEK-ERK pathway signalling, relative to a reference sample or reference value, prior to treatment with an anti-GREM1 antagonist as described herein.

Other exocrine pancreatic cancers include squamous cell carcinomas, which form in pancreatic ducts; adenosquamous carcinomas; signet ring cell carcinomas; and colloid carcinomas, which typically develop from intraductal papillary mucinous neoplasms.

A preferred type of pancreatic cancer for treatment may be resistant to one or more known anti-cancer agents (such as chemotherapy). The pancreatic cancer may be a disseminated pancreatic cancer. The pancreatic cancer may be metastatic pancreatic cancer. Metastatic cancer should be understood as one that has spread from its initial site of origin within the body. Thus, metastatic pancreatic cancer refers to a cancer that starts in the pancreas and spreads to other organs, such as the lungs, liver, bones and brain. Typically, metastatic pancreatic cancer refers to a cancer that starts in the pancreas and spreads to the lungs and/or liver. The pancreatic cancer may also be recurrent pancreatic cancer. In other words, the pancreatic cancer to be treated by the methods of the present invention includes pancreatic cancer that has returned after months or even years after earlier treatment, such as chemotherapy, radiotherapy or curative surgery. A preferred type of pancreatic cancer for treatment may be resistant to one or more known anti-cancer agents (such as chemotherapeutic agents), as described further below.

In some aspects, the pancreatic cancer may be a pancreatic cancer that is responsive to treatment with an inhibitor of Ras-Raf-MEK-ERK signalling, for example, a pancreatic cancer that is responsive to treatment with a MEK or ERK inhibitor as described herein. The pancreatic cancer may alternatively be a pancreatic cancer that is poorly responsive, non-responsive or refractory to treatment with an inhibitor of Ras-Raf-MEK-ERK signalling, for example, a pancreatic cancer that is poorly responsive, non-responsive or refractory to treatment with a MEK or ERK inhibitor as described herein. The pancreatic cancer may be previously considered unsuitable for treatment with an inhibitor of Ras-Raf-MEK-ERK signalling, for example a pancreatic cancer that was previously considered unsuitable for treatment with a MEK or ERK inhibitor as described herein. In some instances, the pancreatic cancer may be initially responsive to treatment with the inhibitor of Ras-Raf-MEK-ERK signalling, but develop resistance to the inhibitor of Ras-Raf-MEK-ERK signalling. For example, the pancreatic cancer may be initially responsive to treatment with a MEK or ERK inhibitor, but develop resistance to the MEK or ERK inhibitor as described herein.

### Lung Cancer

In a further aspect, the present invention relates to the treatment or prevention of lung cancer. The lung cancer is most preferably non-small cell lung cancer (NSCLC). The NSCLC may be advanced NSCLC, such as Stage III or IV NSCLC. The NSCLC may be a squamous cell carcinoma, adenocarcinoma or large cell carcinoma. The cancer may be small cell lung cancer. The lung cancer may be unresectable. The lung cancer may be non-angiogenic and/or may exhibit vessel co-option. The lung cancer may comprise alveolar and/or interstitial tumour growth. The lung cancer may be a primary lung cancer or any secondary cancer that has spread to the lung such as a breast or pancreatic cancer. The lung cancer may be a disseminated lung cancer. The lung cancer may be a metastatic lung cancer. The lung cancer may be a lung cancer that is characterised by having overexpression of GREM1. The lung cancer may also be recurrent lung cancer. In other words, the lung cancer to be treated by the methods of the present invention includes lung cancer that has returned after months or even years after earlier treatment, such as chemotherapy, radiotherapy or curative surgery. A preferred type of lung cancer for treatment may be resistant to one or more known anti-cancer agents (such as chemotherapeutic agents), as described further below.

In some aspects, the lung cancer may be a lung cancer that is responsive to treatment with an inhibitor of Ras-Raf-MEK-ERK signalling, for example, a lung cancer that is responsive to treatment with a MEK or ERK inhibitor as described herein. The lung cancer may alternatively be a lung cancer that is poorly responsive, non-responsive or refractory to treatment with an inhibitor of Ras-Raf-MEK-ERK signalling, for example, a lung cancer that is poorly responsive, non-responsive or refractory to treatment with a MEK or ERK inhibitor as described herein. The lung cancer may be previously considered unsuitable for treatment with an inhibitor of Ras-Raf-MEK-ERK signalling, for example, lung cancer that was previously considered unsuitable for treatment with a MEK or ERK inhibitor as described herein. In some instances, the lung cancer may be initially responsive to treatment with the inhibitor of Ras-Raf-MEK-ERK signalling, but develop resistance to the inhibitor of Ras-Raf-MEK-ERK signalling. For example, the lung cancer may be initially responsive to treatment with a MEK or ERK inhibitor, but develop resistance to the MEK or ERK inhibitor as described herein.

In a further aspect, the lung cancer for treatment is a lung cancer having Ras-Raf-MEK-ERK pathway dysregulation and/or mutations in a Ras (such as KRAS, NRAS and/or HRAS) or Raf gene (such as ARAF, BRAF and/or CRAF), as described herein. For example, the lung cancer for treatment may be a non-small cell lung cancer having MAPK/ERK pathway dysregulation and/or mutations in a Ras (such as KRAS, NRAS and/or HRAS) or Raf gene (such as ARAF, BRAF and/or CRAF), as described herein.

In another embodiment, the lung cancer is a lung cancer in which the Ras-Raf-MEK-ERK signalling pathway is dysregulated in response to treatment with/exposure to an anti-GREM1 antagonist as described herein. In particular, the lung cancer may be a lung cancer in which Ras-Raf-MEK-ERK signalling is induced/upregulated following treatment with an anti-GREM1 antagonist. Such a lung cancer may not display Ras-Raf-MEK-ERK pathway dysregulation in the absence of anti-GREM1 antagonist treatment. In other words, the lung cancer may exhibit normal Ras-Raf-MEK-ERK pathway signalling, relative to a reference sample or reference value, prior to treatment with an anti-GREM1 antagonist as described herein.

### Melanoma

In a further aspect, the present invention relates to the treatment or prevention of melanoma. The melanoma may be a cutaneous/skin melanoma, such as a superficial spreading melanoma, a nodular melanoma, a lentigo maligna melanoma or an acral lentiginous melanoma. The skin melanoma may be an amelanotic melanoma, nevoid melanoma, spitzoid melanoma or a desmoplastic melanoma. In some aspects, the melanoma may be an eye/ocular melanoma, such as a conjunctival melanoma, a choroidal melanoma or an iris melanoma. In other aspects, the melanoma may be a mucosal melanoma.

The melanoma may be a metastatic melanoma. The melanoma may be a melanoma that is characterised by having overexpression of GREM1. The melanoma may also be recurrent melanoma. In other words, the melanoma to be treated by the methods of the present invention includes melanoma that has returned after months or even years after earlier treatment, such as chemotherapy, radiotherapy or curative surgery. A preferred type of melanoma for treatment may be resistant to one or more known anti-cancer agents (such as chemotherapeutic agents), as described further below.

In some aspects, the melanoma may be a melanoma that is responsive to treatment with an inhibitor of Ras-Raf-MEK-ERK signalling, for example, a melanoma that is responsive to treatment with a MEK or ERK inhibitor as described herein. The melanoma may alternatively be a melanoma that is poorly responsive, non-responsive or refractory to treatment with an inhibitor of Ras-Raf-MEK-ERK signalling, for example, a melanoma that is poorly responsive, non-responsive or refractory to treatment with a Ras-Raf-MEK-ERK inhibitor as described herein. The melanoma may be previously considered unsuitable for treatment with an inhibitor of Ras-Raf-MEK-ERK signalling, for example a melanoma that was previously considered unsuitable for treatment with a MEK or ERK inhibitor as described herein. In some instances, the melanoma may be initially responsive to treatment with the inhibitor of Ras-Raf-MEK-ERK signalling, but develop resistance to the inhibitor of Ras-Raf-MEK-ERK signalling. For example, the melanoma may be initially responsive to treatment with a MEK or ERK inhibitor, but develop resistance to the MEK or ERK inhibitor as described herein.

In a further aspect, the melanoma for treatment is a melanoma having MAPK/ERK pathway dysregulation and/or mutations in a Ras (such as KRAS, NRAS and/or HRAS) or Raf gene (such as ARAF, BRAF and/or CRAF), as described herein.

In another embodiment, the melanoma is a melanoma in which the MAPK/ERK pathway is dysregulated in response to treatment with/exposure to an anti-GREM1 antagonist as described herein. In particular, the melanoma may be a melanoma in which MAPK/ERK signalling is induced/upregulated following treatment with an anti-GREM1 antagonist. Such a melanoma may not display MAPK/ERK pathway dysregulation in the absence of anti-GREM1 antagonist treatment. In other words, the melanoma may exhibit normal MAPK/ERK pathway signalling, relative to a reference sample or reference value, prior to treatment with an anti-GREM1 antagonist as described herein.

### Colorectal Cancer

The invention relates in one aspect to prevention or treatment of colorectal cancer. By way of background, the intestinal mucosa is a complex ecosystem and the epithelium has an inter-dependent relationship with its microenvironment, particularly the underlying stroma. Mesenchymal-epithelial crosstalk is intimately involved in regulating homeostasis and is dynamically altered in intestinal regeneration and cancer. Cell-signalling networks are the effector pathways of inter-compartmental crosstalk and control epithelial cell fate determination, but can be co-opted and corrupted by the tumour microenvironment in colorectal cancer.

The current chemotherapeutic management of colorectal cancer has not substantially changed for the last 20 years and is predominantly based around the use of combination cytotoxic agents (such as FOLFOX and FOLFIRI regimens http://www.cancerresearchuk.org/about-cancer/cancer-in-general/treatment/cancer-drugs/drugs) against the proliferating tumour epithelium, and resistance to these epithelial targeted agents may arise. It is now more important than ever to identify new therapies for use in colorectal cancer.

A cancer or tumour for treatment is thus colorectal cancer or a colorectal tumour. An especially preferred form of colorectal cancer for treatment is colorectal cancer that is characterised by having overexpression of GREM1 in stromal cells, i.e. stromal GREM1 overexpression. The stromal cells may be cancer associated fibroblasts. A colorectal cancer with stromal GREM1 overexpression may display no epithelial GREM1 overexpression. A colorectal cancer with stromal GREM1 overexpression may comprise stromal Foxl1 overexpression. A particularly suitable form of colorectal cancer for treatment is colorectal cancer that is a mesenchymal subtype colorectal cancer, also described as CMS4 (Guinney et al, Nat Med 2015). Any other subtypes of colorectal cancer may also be treated including any of CMS1, CMS2 and CMS3 as described in Guinney et al supra. A colorectal cancer as described herein may be a proximal colorectal cancer (or a proximal colorectal tumour). The proximal colon is the region of the large bowel upstream of the splenic flexure, meaning the caecum, the ascending colon and the transverse colon. Cancers or tumours in this region are also referred to as right-sided cancers or tumours. The invention may concern treating right-sided colorectal cancer or a right-sided colorectal tumour.

The colorectal cancer may be distal colorectal cancer (or a distal colorectal tumour). The distal colon is the region of the large bowel downstream of the splenic flexure, meaning the descending colon, the sigmoid colon and the rectum. Cancers or tumours in this region are also referred to as left-sided cancers or tumours. The invention may concern treating left-sided colorectal cancer or a left-sided colorectal tumour. A cancer having stromal overexpression of GREM1 may preferably be a sporadic cancer. The sporadic cancer may be caused by a somatic mutation. The sporadic cancer may be caused by a carcinogenic agent. A sporadic cancer is not due to an inherited genetic mutation. The sporadic cancer may cause the stromal overexpression of GREM1. Proliferation of the sporadic cancer may be dependent on the stromal overexpression of GREM1 in the cancer.

At least three single nucleotide polymorphisms (SNPs) close to GREM1 are independently associated with risk of colorectal cancer (CRC) in white northern Europeans, and probably in other ethnic groups (Tomlinson et al, PLos Genet, 2011). There is a direct link with GREM1 expression and it is likely that the other SNPs have similar effects. In addition, two common SNPs near BMP2, two near BMP4 and one near BMP7 influence the expression of BMP ligands and affect CRC risk. Thus, the cancer may comprise one or more of the above SNPs.

A further type of cancer or tumour for treatment according to the invention is one that exhibits overexpression of GREM1 in epithelial cells. The overexpression of GREM1 in epithelial cells may cause the cancer. Proliferation of the cancer may be dependent on the epithelial overexpression of GREM1. Thus, the cancer may be of epithelial origin. The cancer may be colorectal cancer or duodenal cancer. The cancer may be GREM1-initiated. By GREM1-initiated it is meant that a mutagenic event enhancing activity or expression of GREM1 is causative of the cancer. Such a cancer may be due to an inherited genetic mutation. The cancer may thus be a familial cancer (see below).

A preferred type of colorectal cancer for treatment may be resistant to one or more known anti-cancer agents (such as chemotherapeutic agents), as described further below.

The colorectal cancer may be a disseminated colorectal cancer. The colorectal cancer may be a metastatic colorectal cancer. The colorectal cancer may be metastatic colorectal cancer of the lung. The colorectal cancer may be metastatic colorectal cancer of the liver. The colorectal cancer may be metastatic colorectal cancer of the bone.

The colorectal cancer may be characterised by stromal overexpression of the Fox1. The colorectal cancer may be characterised by stromal overexpression of one or more Wnt ligand. For example, the colorectal cancer may be characterised by stromal overexpression of Wnt5A and/or Wnt2B. A colorectal cancer may be particularly suitable for prevention or treatment using a GREM1 antagonist if said colorectal cancer has stromal overexpression of Foxl1 and/or a Wnt ligand e.g. Wnt5A or Wnt2B.

In some aspects, the colorectal cancer may be a colorectal cancer that is responsive to treatment with an inhibitor of Ras-Raf-MEK-ERK signalling, for example, a colorectal cancer that is responsive to treatment with a MEK or ERK inhibitor as described herein. The colorectal cancer may alternatively be a colorectal cancer that is poorly responsive, non-responsive or refractory to treatment with an inhibitor of Ras-Raf-MEK-ERK signalling, for example, a colorectal cancer that is poorly responsive, non-responsive or refractory to treatment with a MEK or ERK inhibitor as described herein. The colorectal cancer may be previously considered unsuitable for treatment with an inhibitor of Ras-Raf-MEK-ERK signalling, for example, a colorectal cancer that was previously considered unsuitable for treatment with a MEK or ERK inhibitor as described herein. In some instances, the colorectal cancer may be initially responsive to treatment with the inhibitor of Ras-Raf-MEK-ERK signalling, but develop resistance to the inhibitor of Ras-Raf-MEK-ERK signalling. For example, the colorectal caner may be initially responsive to treatment with a MEK or ERK inhibitor as described herein, but develop resistance to the MEK or ERK inhibitor.

In a further aspect, the colorectal cancer for treatment is a colorectal cancer having Ras-Raf-MEK-ERK pathway dysregulation and/or mutations in a Ras (such as KRAS, NRAS and/or HRAS) or Raf gene (such as ARAF, BRAF and/or CRAF), as described herein. For example, the colorectal cancer may be a KRAS/BRAF mutated colorectal cancer. The KRAS mutated colorectal cancer may be characterised as having any of the following mutations in KRAS: G12D, G12V, G12C, G12A, G13D, Q61H, Q61L, Q61R, A146T and/or A146V. The BRAF mutated colorectal cancer may be a BRAF^{V600E}mutation.

In another embodiment, the colorectal cancer is a colorectal cancer in which the Ras-Raf-MEK-ERK pathway is dysregulated in response to treatment with/exposure to an anti-GREM1 antagonist as described herein. In particular, the colorectal cancer may be a colorectal cancer in which Ras-Raf-MEK-ERK signalling is induced/upregulated following treatment with an anti-GREM1 antagonist. Such a colorectal cancer may not display Ras-Raf-MEK-ERK pathway dysregulation in the absence of anti-GREM1 antagonist treatment. In other words, the colorectal cancer may exhibit normal Ras-Raf-MEK-ERK pathway signalling, relative to a reference sample or reference value, prior to treatment with an anti-GREM1 antagonist as described herein.

### Familial Cancer

Familial cancers include cancers resulting from a mutation or mutations in the GREM1 encoding gene, or any other mutation affecting expression of the GREM1 gene. The autosomal dominant condition Hereditary Mixed Polyposis Syndrome (HMPS) is caused by a 40kb duplication upstream of GREM1 that results in a pathological compartment expression switch from a restricted mesenchymal gradient to ectopic GREM1 gene expression throughout the epithelium.

The subject to be treated with anti-GREM1 antagonist may have been previously determined as being at risk of developing a familial cancer. For example, the subject may have been determined as being at risk on the basis of their family history and/or because the subject carries a mutation in a gene known to give rise to, or increase the risk of developing, the familial cancer.

The familial cancer may be Lynch syndrome, which is also referred to as hereditary nonpolyposis colorectal cancer (HNPCC). The familial cancer may be familial adenomatous polyposis (FAP).

Patients or subjects suffering with familial adenomatous polyposis (FAP) may be particularly suitable for treatment with the combination therapy comprising the anti-GREM1 antagonist. The familial cancer to be treated or prevented with the combination therapy comprising the anti-GREM1 antagonist (e.g. an anti-GREM1 antibody) and the inhibitor of Ras-Raf-MEK-ERK signalling may be FAP. A subject who has previously suffered from FAP may be preventatively administered with an anti-GREM1 antagonist in combination with the inhibitor of Ras-Raf-MEK-ERK signalling, e.g. to prevent relapse. A subject who has not previously suffered from FAP but has been previously determined to be at risk of developing FAP may be preventatively administered with an anti-GREM1 antagonist in combination with the inhibitor of Ras-Raf-MEK-ERK signalling. A subject may have been determined as being at risk of developing FAP because it has been found that the subject carries a deleterious mutation in their Apc gene.

In some aspects, the familial cancer may be a familial cancer that is responsive to treatment with an inhibitor of Ras-Raf-MEK-ERK signalling, for example, a familial cancer that is responsive to treatment with a MEK or ERK inhibitor as described herein. The familial cancer may alternatively be a familial cancer that is poorly responsive, non-responsive or refractory to treatment with an inhibitor of Ras-Raf-MEK-ERK signalling, for example, a familial cancer that is poorly responsive, non-responsive or refractory to treatment with a MEK or ERK inhibitor as described herein. The familial cancer may be previously considered unsuitable for treatment with an inhibitor of Ras-Raf-MEK-ERK signalling for example, the familial cancer may have been previously considered unsuitable for treatment with a MEK or ERK inhibitor as described herein. In some instances, the familial cancer may be initially responsive to treatment with the inhibitor of Ras-Raf-MEK-ERK signalling, but develop resistance to the inhibitor of Ras-Raf-MEK-ERK signalling. For example, the familial cancer may be initially responsive to treatment with the MEK or ERK inhibitor as described herein, but develop resistance to the MEK or ERK inhibitor.

In a further aspect, the familial cancer for treatment is a familial cancer having Ras-Raf-MEK-ERK pathway dysregulation and/or mutations in a Ras (such as KRAS, NRAS and/or HRAS) or Raf gene (such as ARAF, BRAF and/or CRAF), as described herein.

In another embodiment, the familial cancer is a familial cancer in which the Ras-Raf-MEK-ERK pathway is dysregulated in response to treatment with/exposure to an anti-GREM1 antagonist as described herein. In particular, the familial cancer may be a familial cancer in which Ras-Raf-MEK-ERK signalling is induced/upregulated following treatment with an anti-GREM1 antagonist. Such a familial cancer may not display Ras-Raf-MEK-ERK pathway dysregulation in the absence of anti-GREM1 antagonist treatment. In other words, the familial cancer may exhibit normal Ras-Raf-MEK-ERK pathway signalling, relative to a reference sample or reference value, prior to treatment with an anti-GREM1 antagonist as described herein.

### Multiple Myeloma

The invention relates in another aspect to treatment or prevention of multiple myeloma. Multiple Myeloma (MM) is a haematological malignancy characterised by the clonal proliferation of plasma cells (PCs) within the bone marrow (BM). It is well known that the BM supports tumour growth in MM, with bi-directional signalling between the tumour cells and the BM critical for the continued growth, spread and survival of the MM PCs. Cellular and non-cellular BM components exert different effects upon the growth and spread of MM PC. While recent studies have identified components of the BM that play a role in disease progression, and therapies targeting these have been developed, the standard-of-care treatments in MM still rely primarily on targeting the tumour cells themselves. While such therapies are effective in prolonging patient survival, due to the large role the BM plays in the growth, spread, survival and drug resistance of MM cells, more effective therapies that target this important aspect of disease are needed. Indeed, MM is a largely incurable disease, with disease relapse a key issue faced in effectively treating this disease.

The invention accordingly is also directed to treatment or prevention of multiple myeloma. Multiple myeloma typically comprises the presence of more than one mass of plasma cells within the bone marrow. Multiple myeloma is thus typically associated with aberrant proliferation of plasma cells in the bone marrow. An especially preferred form of multiple myeloma for treatment is characterised by having overexpression of GREM1 in the bone marrow. The multiple myeloma may therefore comprise stromal GREM1 overexpression. The stromal GREM1 overexpression may be present in the compact bone compartment of the bone. The stromal GREM1 overexpression may reflect an increased number of stromal cells, or an increase in the expression levels of GREM1 within existing GREM1-expressing stromal cells. The bone marrow may comprise osteochrondroreticular (OCR) stem cells. The stromal cells overexpressing GREM1 may comprise OCR stem cells. A preferred type of multiple myeloma for treatment may be resistant to one or more known anti-cancer agents (such as chemotherapeutic agents), as described further below.

In some aspects, the multiple myeloma may be a multiple myeloma that is responsive to treatment with an inhibitor of Ras-Raf-MEK-ERK signalling, for example a multiple myeloma that is responsive to treatment with a MEK or ERK inhibitor as described herein. The multiple myeloma may alternatively be a multiple myeloma that is poorly responsive, non-responsive or refractory to treatment with an inhibitor of Ras-Raf-MEK-ERK signalling, for example a multiple myeloma that is poorly responsive, non-responsive or refractory to treatment with a MEK or ERK inhibitor as described herein. The multiple myeloma may be previously considered unsuitable for treatment with an inhibitor of Ras-Raf-MEK-ERK signalling, for example a multiple myeloma that was previously considered unsuitable for treatment with a MEK or ERK inhibitor. In some instances, the multiple myeloma may be initially responsive to treatment with the inhibitor of Ras-Raf-MEK-ERK signalling, but develop resistance to the inhibitor of Ras-Raf-MEK-ERK signalling. For example, the multiple myeloma may be initially responsive to treatment with the MEK or ERK inhibitor as described herein, but develop resistance to the MEK or ERK inhibitor.

In a further aspect, the multiple myeloma for treatment is a multiple myeloma having Ras-Raf-MEK-ERK pathway dysregulation and/or mutations in a Ras (such as KRAS, NRAS and/or HRAS) or Raf gene (such as ARAF, BRAF and/or CRAF), as described herein.

In another embodiment, the multiple myeloma is a multiple myeloma in which the Ras-Raf-MEK-ERK pathway is dysregulated in response to treatment with/exposure to an anti-GREM1 antagonist as described herein. In particular, the multiple myeloma may be a multiple myeloma in which Ras-Raf-MEK-ERK signalling is induced/upregulated following treatment with an anti-GREM1 antagonist. Such a multiple myeloma may not display Ras-Raf-MEK-ERK pathway dysregulation in the absence of anti-GREM1 antagonist treatment. In other words, the multiple myeloma may exhibit normal Ras-Raf-MEK-ERK pathway signalling, relative to a reference sample or reference value, prior to treatment with an anti-GREM1 antagonist as described herein.

### Breast Cancer

The invention relates in another aspect to treatment or prevention of breast cancer. The breast cancer may be invasive breast cancer, such as invasive lobular breast cancer. The breast cancer may be triple negative breast cancer. The breast cancer may be inflammatory breast cancer. The breast cancer may be angiosarcoma of the breast. The breast cancer may be ductal carcinoma in situ or lobular carcinoma in situ.

The invention provides for treatment and prevention of breast cancer by administering an anti-GREM1 antagonist in combination with an inhibitor of Ras-Raf-MEK-ERK signalling. The breast cancer may comprise stromal GREM1 overexpression. The stromal breast cells overexpressing GREM1 may comprise stromal fibroblasts, also described herein as cancer-associated fibroblasts. The breast cancer may also be recurrent breast cancer. In other words, the breast cancer to be treated by the methods of the present invention includes breast cancer that has returned after months or even years after earlier treatment, such as chemotherapy, radiotherapy or curative surgery. A preferred type of breast cancer for treatment may be resistant to one or more known anti-cancer agents (such as chemotherapeutic agents), as described further below. The breast cancer may be a disseminated breast cancer. The breast cancer may be a metastatic breast cancer. The breast cancer may be metastatic breast cancer of the lung. The breast cancer may be metastatic breast cancer of the liver. The breast cancer may be metastatic breast cancer of the bone.

In some aspects, the breast cancer may be a breast cancer that is responsive to treatment with an inhibitor of Ras-Raf-MEK-ERK signalling, for example a breast cancer that is responsive to treatment with a MEK or ERK inhibitor as described herein. The breast cancer may alternatively be a breast cancer that is poorly responsive, non-responsive or refractory to treatment with an inhibitor of Ras-Raf-MEK-ERK signalling, for example a breast cancer that is poorly responsive, non-responsive or refractory to treatment with a MEK or ERK inhibitor as described herein. The breast cancer may be previously considered unsuitable for treatment with an inhibitor of Ras-Raf-MEK-ERK signalling, for example a breast cancer that was previously considered unsuitable for treatment with a MEK or ERK inhibitor as described herein. In some instances, the breast cancer may be initially responsive to treatment with an inhibitor of Ras-Raf-MEK-ERK signalling, but develop resistance to the inhibitor of Ras-Raf-MEK-ERK signalling. For example, the breast cancer may be initially responsive to treatment with the MEK or ERK inhibitor as described herein, but develop resistance to the MEK or ERK inhibitor.

In another embodiment, the breast cancer is a breast cancer in which the Ras-Raf-MEK-ERK pathway is dysregulated in response to treatment with/exposure to an anti-GREM1 antagonist as described herein. In particular, the breast cancer may be a breast cancer in which Ras-Raf-MEK-ERK signalling is induced/upregulated following treatment with an anti-GREM1 antagonist. Such a breast cancer may not display Ras-Raf-MEK-ERK pathway dysregulation in the absence of anti-GREM1 antagonist treatment. In other words, the breast cancer may exhibit normal Ras-Raf-MEK-ERK pathway signalling, relative to a reference sample or reference value, prior to treatment with an anti-GREM1 antagonist as described herein.

### Prostate Cancer

In a further aspect, the present invention relates to the treatment or prevention of prostate cancer. The prostate cancer may be prostatic adenocarcinoma. The prostate cancer may be a transitional cell carcinoma or urothelial cancer that has spread to the prostate.

The prostate cancer may be a prostate cancer that is characterised by having overexpression of GREM1. The prostate cancer may also be recurrent prostate cancer. In other words, the prostate cancer to be treated by the methods of the present invention includes prostate cancer that has returned after months or even years after earlier treatment, such as chemotherapy, radiotherapy or curative surgery. A preferred type of prostate cancer for treatment may be resistant to one or more known anti-cancer agents (such as chemotherapeutic agents), as described further below.

The present invention further provides for treatment and prevention of prostate cancer by administering an anti-GREM1 antagonist in combination with an inhibitor of Ras-Raf-MEK-ERK signalling. The prostate cancer may be a disseminated prostate cancer. The prostate cancer may be a metastatic prostate cancer. The prostate cancer may be metastatic prostate cancer of the lung. The prostate cancer may be metastatic prostate cancer of the liver. The prostate cancer may be metastatic prostate cancer of the bone.

In some aspects, the prostate cancer may be a prostate cancer that is responsive to treatment with an inhibitor of Ras-Raf-MEK-ERK signalling, for example a prostate cancer that is responsive to treatment with a MEK or ERK inhibitor as described herein. The prostate cancer may alternatively be a prostate cancer that is poorly responsive, non-responsive or refractory to treatment with an inhibitor of Ras-Raf-MEK-ERK signalling, for example a prostate cancer that is poorly responsive, non-responsive or refractory to treatment with a MEK or ERK inhibitor as described herein. The prostate cancer may be previously considered unsuitable for treatment with an inhibitor of Ras-Raf-MEK-ERK signalling, for example a prostate cancer that was previously considered unsuitable for treatment with a MEK or ERK inhibitor as described herein. In some instances, the prostate cancer may be initially responsive to treatment with an inhibitor of Ras-Raf-MEK-ERK signalling, but develop resistance to the inhibitor of Ras-Raf-MEK-ERK signalling. For example, the prostate cancer may be initially responsive to treatment with a MEK or ERK inhibitor as described herein, but develop resistance to the MEK or ERK inhibitor.

In another embodiment, the prostate cancer is a prostate cancer in which the Ras-Raf-MEK-ERK pathway is dysregulated in response to treatment with/exposure to an anti-GREM1 antagonist as described herein. In particular, the prostate cancer may be a prostate cancer in which Ras-Raf-MEK-ERK signalling is induced/upregulated following treatment with an anti-GREM1 antagonist. Such a prostate cancer may not display Ras-Raf-MEK-ERK pathway dysregulation in the absence of anti-GREM1 antagonist treatment. In other words, the prostate cancer may exhibit normal Ras-Raf-MEK-ERK pathway signalling, relative to a reference sample or reference value, prior to treatment with an anti-GREM1 antagonist as described herein.

### Bladder Cancer

In a further aspect, the present invention relates to the treatment or prevention of bladder cancer. The bladder cancer may be transitional cell (urothelial) bladder cancer. The bladder cancer may arise from the epithelial lining of the urinary bladder. The bladder cancer may be non-muscle invasive bladder cancer. The bladder cancer may be squamous cell bladder cancer. The bladder cancer may be an adenocarcinoma. The bladder cancer may be a high grade T1 tumour that has grown from the bladder lining into the lamina propria. The bladder cancer may be a superficial cancer or an invasive bladder cancer. The bladder cancer may be a recurrent bladder cancer. The term recurrent bladder cancer as used herein refers to a bladder cancer that has recurred following treatment, such as surgical treatment.

The present invention further provides for treatment and prevention of bladder cancer by administering an anti-GREM1 antagonist in combination with an inhibitor of Ras-Raf-MEK-ERK signalling. The bladder cancer may be a disseminated bladder cancer. The bladder cancer may be a metastatic bladder cancer. The bladder cancer may be metastatic bladder cancer of the lung. The bladder cancer may be metastatic bladder cancer of the liver. The bladder cancer may be metastatic bladder cancer of the bone. The bladder cancer may be a bladder cancer that is characterised by having overexpression of GREM1. The bladder cancer may also be recurrent bladder cancer. In other words, the bladder cancer to be treated by the methods of the present invention includes bladder cancer that has returned after months or even years after earlier treatment, such as chemotherapy, radiotherapy or curative surgery. A preferred type of bladder cancer for treatment may be resistant to one or more known anti-cancer agents (such as chemotherapeutic agents), as described further below.

In some aspects, the bladder cancer may be a bladder cancer that is responsive to treatment with an inhibitor of Ras-Raf-MEK-ERK signalling, for example a bladder cancer that is responsive to treatment with a MEK or ERK inhibitor as described herein. The bladder cancer may alternatively be a bladder cancer that is poorly responsive, non-responsive or refractory to treatment with an inhibitor of Ras-Raf-MEK-ERK signalling, for example, a bladder cancer that is poorly responsive, non-responsive or refractory to treatment with a MEK or ERK inhibitor. The bladder cancer may be previously considered unsuitable for treatment with an inhibitor of Ras-Raf-MEK-ERK signalling, for example a bladder cancer that was previously considered unsuitable for treatment with a MEK or ERK inhibitor as described herein. In some instances, the bladder cancer may be initially responsive to treatment with an inhibitor of Ras-Raf-MEK-ERK signalling, but develop resistance to the inhibitor of Ras-Raf-MEK-ERK signalling. For example, the bladder cancer may be initially responsive to treatment with a MEK or ERK inhibitor as described herein, but develop resistance to the MEK or ERK inhibitor.

In another embodiment, the bladder cancer is a bladder cancer in which the Ras-Raf-MEK-ERK pathway is dysregulated in response to treatment with/exposure to an anti-GREM1 antagonist as described herein. In particular, the bladder cancer may be a bladder cancer in which Ras-Raf-MEK-ERK signalling is induced/upregulated following treatment with an anti-GREM1 antagonist. Such a bladder cancer may not display Ras-Raf-MEK-ERK pathway dysregulation in the absence of anti-GREM1 antagonist treatment. In other words, the bladder cancer may exhibit normal Ras-Raf-MEK-ERK pathway signalling, relative to a reference sample or reference value, prior to treatment with an anti-GREM1 antagonist as described herein.

### Ovarian Cancer

In a further aspect, the present invention relates to the treatment or prevention of ovarian cancer. The ovarian cancer may be epithelial ovarian cancer, germ cell ovarian cancer or sex cord stromal ovarian cancer. The ovarian cancer may be primary peritoneal cancer. The ovarian cancer may be fallopian tube cancer. The ovarian cancer may be characterised by borderline ovarian tumours. The ovarian cancer may be characterised by germ cell ovarian tumours. The ovarian cancer may be clear cell ovarian cancer. The cancer may be serous ovarian cancer. The ovarian cancer may be mucinous ovarian cancer. The ovarian cancer may be endometrioid cancer.

The ovarian cancer may be an ovarian cancer that is characterised by having overexpression of GREM1. The ovarian cancer may also be recurrent ovarian cancer. In other words, the ovarian cancer to be treated by the methods of the present invention includes ovarian cancer that has returned after months or even years after earlier treatment, such as chemotherapy, radiotherapy or curative surgery. A preferred type of ovarian cancer for treatment may be resistant to one or more known anti-cancer agents (such as chemotherapeutic agents), as described further below.

The present invention further provides for treatment and prevention of ovarian cancer by administering an anti-GREM1 antagonist in combination with an inhibitor of Ras-Raf-MEK-ERK signalling. The ovarian cancer may be a disseminated ovarian cancer. The ovarian cancer may be a metastatic ovarian cancer. The ovarian cancer may be metastatic ovarian cancer of the lung. The ovarian cancer may be metastatic ovarian cancer of the liver. The ovarian cancer may be metastatic ovarian cancer of the bone.

In some aspects, the ovarian cancer may be an ovarian cancer that is responsive to treatment with an inhibitor of Ras-Raf-MEK-ERK signalling, for example an ovarian cancer that is responsive to treatment with a MEK or ERK inhibitor as described herein. The ovarian cancer may alternatively be an ovarian cancer that is poorly responsive, non-responsive or refractory to treatment with an inhibitor of Ras-Raf-MEK-ERK signalling, for example an ovarian cancer that is poorly responsive, non-responsive or refractory to treatment with a MEK or ERK inhibitor as described herein. The ovarian cancer may be previously considered unsuitable for treatment with an inhibitor of Ras-Raf-MEK-ERK signalling, for example an ovarian cancer that was previously considered unsuitable for treatment with a MEK or ERK inhibitor as described herein. In some instances, the ovarian cancer may be initially responsive to treatment with an inhibitor of Ras-Raf-MEK-ERK signalling, but develop resistance to the inhibitor of Ras-Raf-MEK-ERK signalling. For example, the ovarian cancer may be initially responsive to treatment with a MEK or ERK inhibitor as described herein, but develop resistance to the MEK or ERK inhibitor.

In another embodiment, the ovarian cancer is an ovarian cancer in which the Ras-Raf-MEK-ERK pathway is dysregulated in response to treatment with/exposure to an anti-GREM1 antagonist as described herein. In particular, the ovarian cancer may be an ovarian cancer in which Ras-Raf-MEK-ERK signalling is induced/upregulated following treatment with an anti-GREM1 antagonist. Such an ovarian cancer may not display Ras-Raf-MEK-ERK pathway dysregulation in the absence of anti-GREM1 antagonist treatment. In other words, the ovarian cancer may exhibit normal Ras-Raf-MEK-ERK pathway signalling, relative to a reference sample or reference value, prior to treatment with an anti-GREM1 antagonist as described herein.

### Stroma and Epithelium

The cancers described for prevention or treatment herein using a GREM1 antagonist in combination with an inhibitor of MAPK/ERK signalling may comprise stromal and/or epithelial GREM1 overexpression.

The terms “stromal cell(s)” or “stroma” as used herein refers to structural and/or connective portions of a tissue or organ.

Stromal tissue is primarily made of extracellular matrix containing connective tissue cells. Extracellular matrix is primarily composed of ground substance—a porous, hydrated gel, made mainly from proteoglycan aggregates—and connective tissue fibers. There are three types of fibers commonly found within the stroma: collagen type I, elastic, and reticular (collagen type III) fibres. Fibroblasts and pericytes are among the most common types of stromal cells.

In the context of a cancer or tumour (e.g. initiating in the epithelium of a tissue or organ), the stroma of the tissue or organ may assist cancer growth and progression. The stroma associated with the cancer or tumour may be a desmoplastic stroma caused by growth of fibrous or connective tissue around the cancer or tumour.

The overexpression of GREM1 may be observed in any part of the stroma/any stromal cells. The stromal cells may be fibroblasts or fibroblast-like support cell. The stromal cells may be fibroblasts or fibroblast-like support cell isolated from a desmoplastic stroma of any cancer or tumour described above, such as from the pancreas, colon or rectum in a colorectal cancer, or the bone marrow in multiple myeloma. The stromal cells may be cancer-associated fibroblasts.

The term “epithelial” as used herein refers to a cell derived from the outer or inner linings of a tissue or organ. In relation to the colon, the intestinal epithelium is the layer of cells that form the luminal surface or lining of both the small and large intestine of the gastrointestinal tract. It is composed of simple columnar epithelium. The “upper barrier” is the intestinal epithelial single layer of columnar cells consisting of four intestinal epithelial cell types: the absorbent enterocytes, the goblet cells, the Paneth cells and the enteroendocrine cells. Upper barrier features are similar in small and large bowel. The main difference is constituted by the presence of elevations and projections (circular folds, villi and microvilli) in duodenum, jejunum and ileum that allows the increase of the absorption surface. This is not observed in the colon, which instead shows a flat surface. Amongst the mucous membrane protrusions termed villi, there are inflections called crypts of Lieberkuhn, which are distinct glandular invaginations. The cell in which epithelial GREM1 overexpression is observed may be any epithelial cell, such as any intestinal epithelial cell.

While not being bound by theory, the present inventors postulate that overexpression of GREM1 in the epithelium and/or stroma may promote a stem/progenitor cell phenotype (increasing the number of stem/progenitor cells), promoting epithelial stem cell behaviour and driving cancer progression and/or resistance to chemotherapeutic agents. Thus, a combination therapy comprising a GREM1 antagonist and an inhibitor of Ras-Raf-MEK-ERK signalling used according to the invention may prevent induction of an aberrant cancer stem/progenitor cell phenotype, reduce epithelial stem cell behaviour and/or decrease the number of stem/progenitor cells, in the epithelium of a tissue or organ of a subject in which cancer is to be prevented or treated. The ability of a GREM1 antagonist to affect stem cell behaviour may be assayed clinically by the assessment of known epithelial and cancer stem cell markers.

The overexpression of GREM1 in the stroma and/or epithelium may be determined by any means. The overexpression of GREM1 is typically determined by comparison to the level of the marker in normal cells of the same tissue type, i.e. basal expression level. The expression is typically normalized against the expression level of other genes, preferably comprising one or more housekeeping genes. GREM1 may also be classified as showing an overexpression or underexpression in a threshold percentage of a population of cancer patients. The overexpression in each patient in the population may be higher than 2 from the geometric mean. At least 10%, more preferably at least 15% or more of the patients in the population may display such an overexpression.

GREM1 stromal overexpression refers to stromal GREM1 levels being higher than that of a matched normal tissue. For example, stromal GREM1 levels may be at least two fold higher than that of matched normal tissue.

Where GREM1 is overexpressed, its amount may be increased by any amount relative to basal. For example, GREM1-initiated cancers such as HMPS may comprise a several thousand-fold upregulation of epithelial GREM1, whereas no GREM1 expression is observed in normal epithelium. Sporadic cancers comprising stromal GREM overexpression may comprise any level of stromal overexpression over the physiological GREM1 expression level in normal stroma of the organ. The skilled person is able to evaluate the existence of an overexpression in stroma or epithelium compared with the level of GREM1 in normal cells of the same type.

The amount determined may be the amount of mRNA. The cancer may thus comprise an overexpression of GREM1 mRNA. The cancer may comprise an increased amount of GREM1 mRNA compared with normal cells of the same tissue type. The mRNA may be increased by any amount. The amount of mRNA can be measured using a quantitative reverse transcription polymerase chain reaction (qRT-PCR), such as real time qRT-PCR, quantigene assay (Affymetrix/Thermo Fisher), by northern blotting or using microarrays, RNA sequencing. mRNA expression is preferably determined by comparing the gene expression of a sample to the distribution of expression levels of the specific gene across a reference sample composed of tumours that are diploid for that gene. A z-score may be derived using RNAseq by expectation maximisation (RSEM) algorithm (cBioportal for Cancer Genomics, www.cbioportal.org; Gao et al, 2013 and Serami eta al 2012). A z-score of 2 SD higher or lower than the mean of the reference set is preferably considered as overexpression or underexpression respectively.

The amount determined may be the amount of protein. The cancer may comprise an overexpression of GREM1 protein, such as compared with normal cells of the same tissue type. The protein may be increased by any amount. The amount of protein can be measured using immunohistochemistry, western blotting, mass spectrometry or fluorescence-activated cell sorting (FACS), including by use of an anti-GREM1 antibody of the invention. The thresholds for determining expression may vary between techniques used, and may be validated against immunohistochemistry scores.

The use of GREM1 antagonists in combination with an inhibitor of Ras-Raf-MEK-ERK signalling for treating or preventing cancer in a patient as described herein may thus comprise (a) measuring the amount of GREM1 in the cancer and (b) if the cancer comprises an overexpression of GREM1, administering to the patient the GREM1 antagonist in combination with the inhibitor of Ras-Raf-MEK-ERK signalling and thereby treating or preventing the cancer. The amount of GREM1 may be the mRNA or protein amount, and the overexpression may be any overexpression discussed above.

The above measurements may be carried out in any suitable sample from the patient. The measurements may be carried out in a cancer or tumour biopsy obtained from the patient. The stroma and/or epithelium (stromal and/or epithelial cells) may be isolated from the biopsy. The biopsy tissue may be formalin fixed paraffin embedded (FFPE) tissue or fresh tissue. The tissue may be pancreatic tissue, bladder tissue, lung tissue, endometrial tissue, breast tissue, stomach tissue, duodenal tissue, oesophageal tissue, bone marrow or colorectal tissue. Any of the methods discussed above may be carried out on the cancer biopsy. Such methods may also be carried out on cancer cells circulating in the blood of the patient. The RNA methods may be carried out on urinary or blood exosomes.

Cancer Associated with Ras-Raf-MEK-ERK Signalling Pathway Dysregulation

As noted above, the inventors have demonstrated that KRAS signalling is upregulated in a KPC mouse model following exposure with an anti-GREM1 antagonist (Ab7326) according to the invention. Moreover, genes involved in the Ras-Raf-MEK-ERK pathway, such as Mapk1, Nras and Myc are up-regulated upon exposure with Ab7326 in a KPC mouse model. Thus, a combination therapy targeting both Gremlin-1 and the Ras-Raf-MEK-ERK pathway would be advantageous.

The invention encompasses the use of an anti-GREM1 antagonist in combination with an inhibitor of Ras-Raf-MEK-ERK signalling for the treatment or prevention of a cancer associated with Ras-Raf-MEK-ERK pathway dysregulation and/or a cancer containing a mutation in a Ras or Raf gene. The Ras gene may, for example, be KRAS, NRAS and/or HRAS. The Raf gene may, for example, be ARAF, BRAF and/or CRAF. Cancers associated with MAPK/ERK pathway dysregulation and/or cancers containing a mutation in a Ras or Raf gene may include, for example, colorectal cancer, multiple myeloma, pancreatic cancer, bladder cancer, breast cancer, lung cancer, stomach cancer, ovarian cancer, duodenal cancer, oesophageal cancer, head and neck cancer, prostate cancer, glioma, endometrial cancer, liver cancer, spleen cancer, bone-resident cancer, and osteosarcoma.

The invention also encompasses a combination therapy comprising an anti-GREM1 antagonist as defined herein and an inhibitor of Ras-Raf-MEK-ERK signalling for the treatment or prevention of cancers in which the Ras-Raf-MEK-ERK pathway is dysregulated following earlier exposure to/treatment with an anti-GREM1 antagonist. In particular, the cancer may be a cancer in which Ras-Raf-MEK-ERK pathway signalling is induced/upregulated following treatment with an anti-GREM1 antagonist as described herein. Such a cancer may not display Ras-Raf-MEK-ERK pathway dysregulation in the absence of anti-GREM1 antagonist treatment. In other words, the cancer may exhibit normal Ras-Raf-MEK-ERK pathway signalling, relative to a reference sample or reference value, prior to treatment with an anti-GREM1 antagonist as described herein.

In a preferred embodiment, the invention is directed to the treatment or prevention of a pancreatic cancer. For example, the invention encompasses a combination therapy comprising an anti-GREM1 antagonist as defined herein and an inhibitor of Ras-Raf-MEK-ERK signalling for the treatment or prevention of pancreatic cancers in which the Ras-Raf-MEK-ERK pathway is dysregulated following earlier exposure to/treatment with an anti-GREM1 antagonist. Pancreatic cancer may alternatively or additionally be characterised by constitutive activation of the classical MAPK pathway. In many cases, pancreatic cancer involves gain-of-function mutations in KRAS, which encodes RAS. Typical gain of function mutations include G12D or G12R substitutions and are seen in pancreatic ductal adenocarcinoma. In other cases, pancreatic cancer involves gain-of-function mutations in BRAF, which encodes B-RAF. A typical gain of function mutation is seen at position V600E, resulting in constitutive kinase activity of RAF.

As noted above, the invention may also be directed to the treatment or prevention of a KRAS/BRAF mutated colorectal cancer. In another aspect, the invention is directed to a BRAF-mutated melanoma, such as a melanoma harbouring a V600E mutation in BRAF.

### GREM1

The terms GREM1 or Gremlin-1 as used in the present invention in the context of a protein refer to a protein that typically has the amino acid sequence as set out in the UniProt entry 060565 (SEQ ID NO: 1), human GREM1. The terms GREM1 and Gremlin-1 may also refer to a Gremlin-1 polypeptide which:
(a) comprises or consists of the amino acid sequence of SEQ ID NO: 1 with or without the N-terminal signal peptide, i.e. may comprise or consist of the mature peptide sequence as shown in SEQ ID NO: 21; or
(b) is a derivative having one or more amino acid substitutions, modifications, deletions or insertions relative to the amino acid sequence of SEQ ID NO: 1 with or without the N-terminal signal peptide (as shown in SEQ ID NO: 21), which retains the activity of Gremlin-1, such as the amino acid sequence of SEQ ID NO: 20.
(c) a variant thereof, such variants typically retain at least about 60%, 70%, 80%, 90%, 91%, 92%, 93%, 94% or 95% identity to SEQ ID NO: 1 (or SEQ ID NO: 20 or 21) (or even about 96%, 97%, 98% or 99% identity). In other words, such variants may retain about 60%—about 99% identity to SEQ ID NO: 1, suitably about 80%—about 99% identity to SEQ ID NO: 1, more suitably about 90%—about 99% identity to SEQ ID NO: 1 and most suitably about 95%—about 99% identity to SEQ ID NO: 1. Variants are described further below.

As discussed further below, residue numbers are typically quoted based on the sequence of SEQ ID NO: 1. However, residue numbering could readily be extrapolated by the skilled person to a derivative or variant sequence as discussed above. Where residue numbers are quoted, the invention also encompasses these residues on a variant or derivative sequence.

A GREM1 or Gremlin-1 nucleic acid sequence may comprise or consist of the sequence of SEQ ID NO: 36 or SEQ ID NO: 37 or a variant thereof. Variant nucleic acid sequences are described further below. A GREM1 or Gremlin-1 nucleic sequence may comprise or consist of any GREM1 transcript variant. Examples of GREM1 transcripts variants are Transcript 1 (NCBI: NM_013372.6; ENSEMBL: ENST00000560677.5); Transcript 2: NCBI: NM_001191323.1; ENSEMBL: ENST00000560830.1); Transcript 3: NCBI: NM_001191322.1; ENSEMBL: ENST00000622074.1. The sequences available at the above accession numbers as of 18 Jun. 2018 are incorporated by reference herein.

### Antagonist

An anti-GREM1 antagonist is any molecule that reduces the function or activity of GREM1. The anti-GREM1 antagonist may reduce function or activity of GREM1 by any amount. The anti-GREM1 antagonist may reduce GREM1 function or activity by at least 10%, at least 20%, at least 30% at least 40%, at least 50%, at least 60%, at least 70%, at least 80%, at least 90% or at least 95%, or may prevent any GREM1 function or activity. The extent to which an anti-GREM1 antagonist reduces GREM1 function or activity may be determined by measuring GREM1 function or activity in cells in the presence and absence of the anti-GREM1 antagonist. The cells may be normal cells or cancer cells. The cells may be cancer cells as described above. They may be pancreatic cancer cells. The pancreatic cancer cells may be present in KPC mouse models as described in the Examples. Thus, an in vivo assay for activity of a GREM1 antagonist in a combination therapy in pancreatic cancer may be performed in a mouse model. More generally, a GREM1 antagonist shown to reduce function or activity of GREM1 by any means may then be assayed in vitro or in vivo for its ability to prevent or reduce proliferation of cancer cells, such as pancreatic cancer cells, or to prevent, reduce or eliminate a cancer or tumour.

The antagonist may decrease GREM1 function by any means. It may increase or decrease the activity or amount of any molecule affecting GREM1 function directly or indirectly. It may decrease the amount of GREM1 at the mRNA or protein level. It may increase degradation of GREM1. It may decrease the function of GREM1 by inhibitory modification. It may decrease the transcription of a molecule enhancing GREM1 function. It may disrupt DNA encoding GREM1 or a molecule enhancing GREM1 function, using an agent such as a zinc finger nuclease.

The antagonist may be an agent interacting with Gremlin-1. An agent that interacts with Gremlin-1 is typically an agent which binds Gremlin-1. Agents that interact with Gremlin-1 may modulate Gremlin-1. An inhibitory modulating agent may have an effect on any of the functions of Gremlin-1, but typically reduces binding of Gremlin-1 to BMP (BMP 2/4/7). The antagonist may be a BMP-7 mimetic molecule Gremlin-1 is a negative regulator of BMP, so reduced binding increases signalling through BMP. An activating modulating agent may increase binding of Gremlin-1 to BMP.

BMP binding and signalling may be detected by any method known in the art.

The antagonist may act by binding the active site of GREM1 or act allosterically by binding at a different site. The antagonist may act by binding a regulator or ligand for GREM1, to thereby reduce activation of GREM1. The antagonist may be reversible or irreversible.

A GREM1 antagonist may be a small molecule inhibitor, a peptide, a protein, an antibody, a polynucleotide, an oligonucleotide, an antisense RNA, small interfering RNA (siRNA) or small hairpin RNA (shRNA).

An antagonist of GREM1 may be an oligonucleotide which specifically hybridises to an mRNA encoding GREM1 or an mRNA encoding a molecule which enhances GREM1 activity. An antagonist of GREM1 may be a polynucleotide encoding any molecule that decreases GREM1 function. For example, the GREM1 antagonist may be a polynucleotide encoding an anti-GREM1 antibody described herein.

An antagonist of GREM1 may be an antibody which specifically binds to any target molecule (typically a protein) so as to decrease GREM1 function directly or indirectly. The antagonist may be an antibody specifically binding GREM1. In this aspect, the antibody may decrease GREM1 function by allosteric inactivation or by blocking interaction between its target and a ligand required for activity.

Interaction of an antagonist agent with protein residues may be determined by any appropriate method known in the art, such as distances between the residue and agent as determined by x-ray crystallography (typically less than 6 A, or less than 4 A). The region of Gremlin-1 which may be targeted by a therapeutic may include amino acids Asp92-Leu99, Arg116-His130, Ser137-Ser142, Cys176-Cys178. These are within 6A of those mutated on the surface of Gremlin-1.

### Antibody Antagonists

The term “antibody” as referred to herein includes whole antibodies and any antigen binding fragment (i.e., “antigen-binding portion”) or single chains thereof. An antibody refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as HCVR or V_{H}) and a heavy chain constant region. Each light chain is comprised of a light chain variable region (abbreviated herein as LCVR or V_{L}) and a light chain constant region. The variable regions of the heavy and light chains contain a binding domain that interacts with an antigen. The V_{H} and V_{L} regions can be further subdivided into regions of hypervariability, termed complementarity determining regions (CDR), interspersed with regions that are more conserved, termed framework regions (FR).

The constant regions of the antibodies may mediate the binding of the immunoglobulin to host tissues or factors, including various cells of the immune system (e.g., effector cells) and the first component (Clq) of the classical complement system.

An antibody used according to the invention may be a monoclonal antibody or a polyclonal antibody, and will typically be a monoclonal antibody. An antibody used according to the invention may be a chimeric antibody, a CDR-grafted antibody, a nanobody, a human or humanised antibody or an antigen-binding portion of any thereof. For the production of both monoclonal and polyclonal antibodies, the experimental animal is typically a non-human mammal such as a goat, rabbit, rat or mouse but the antibody may also be raised in other species.

Polyclonal antibodies may be produced by routine methods such as immunisation of a suitable animal, with the antigen of interest. Blood may be subsequently removed from the animal and the IgG fraction purified.

Antibodies against Gremlin-1 may be obtained, where immunisation of an animal is necessary, by administering the polypeptides to an animal, e.g. a non-human animal, using well-known and routine protocols, see for example Handbook of Experimental Immunology, D. M. Weir (ed.), Vol 4, Blackwell Scientific Publishers, Oxford, England, 1986). Many warm-blooded animals, such as rabbits, mice, rats, sheep, cows, camels or pigs may be immunized. However, mice, rabbits, pigs and rats are generally most suitable.

Monoclonal antibodies may be prepared by any method known in the art such as the hybridoma technique (Kohler & Milstein, 1975, Nature, 256:495-497), the trioma technique, the human B-cell hybridoma technique (Kozbor et al., 1983, Immunology Today, 4:72) and the EBV-hybridoma technique (Cole et al., Monoclonal Antibodies and Cancer Therapy, pp77-96, Alan R Liss, Inc., 1985).

Antibodies used according to the invention may also be generated using single lymphocyte antibody methods by cloning and expressing immunoglobulin variable region cDNAs generated from single lymphocytes selected for the production of specific antibodies by for example the methods described by Babcook, J. et al., 1996, Proc. Natl. Acad. Sci. USA 93(15): 7843-78481; WO92/02551; WO2004/051268 and WO2004/106377.

The antibodies can also be generated using various phage display methods known in the art and include those disclosed by Brinkman et al. (in J. Immunol. Methods, 1995, 182: 41-50), Ames et al. (J. Immunol. Methods, 1995, 184:177-186), Kettleborough et al. (Eur. J. Immunol. 1994, 24:952-958), Persic et al. (Gene, 1997 187 9-18), Burton et al. (Advances in Immunology, 1994, 57:191-280) and WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

Fully human antibodies are those antibodies in which the variable regions and the constant regions (where present) of both the heavy and the light chains are all of human origin, or substantially identical to sequences of human origin, but not necessarily from the same antibody. Examples of fully human antibodies may include antibodies produced, for example by the phage display methods described above and antibodies produced by mice in which the murine immunoglobulin variable and optionally the constant region genes have been replaced by their human counterparts e.g. as described in general terms in EP 0546073, U5,545,806, U.S. Pat. Nos. 5,569,825, 5,625,126, 5,633,425, 5,661,016, 5,770,429, EP 0438474 and EP 0463151.

Alternatively, an antibody used according to the invention may be produced by a method comprising immunising a non-human mammal with a Gremlin-I immunogen; obtaining an antibody preparation from said mammal; deriving therefrom monoclonal antibodies that recognise Gremlin-1.

The antibody molecules used according the present invention may comprise a complete antibody molecule having full length heavy and light chains or a fragment or antigen-binding portion thereof. The term “antigen-binding portion” of an antibody refers to one or more fragments of an antibody that retain the ability to selectively bind to an antigen. It has been shown that the antigen-binding function of an antibody can be performed by fragments of a full-length antibody. The antibodies and fragments and antigen binding portions thereof may be, but are not limited to Fab, modified Fab, Fab′, modified Fab′, F(ab′)₂, Fv, single domain antibodies (e.g. VH or VL or VHH), scFv, bi, tri or tetra-valent antibodies, Bis-scFv, diabodies, triabodies, tetrabodies and epitope-binding fragments of any of the above (see for example Holliger and Hudson, 2005, Nature Biotech. 23(9):1126-1136; Adair and Lawson, 2005, Drug Design Reviews—Online 2(3), 209-217). The methods for creating and manufacturing these antibody fragments are well known in the art (see for example Verma et al., 1998, Journal of Immunological Methods, 216, 165-181). Other antibody fragments for use in the present invention include the Fab and Fab′ fragments described in International patent applications WO 2005/003169, WO 2005/003170 and WO 2005/003171 and Fab-dAb fragments described in International patent application WO2009/040562. Multi-valent antibodies may comprise multiple specificities or may be monospecific (see for example WO 92/22853 and WO 05/113605). These antibody fragments may be obtained using conventional techniques known to those of skill in the art, and the fragments may be screened for utility in the same manner as intact antibodies.

The constant region domains of the antibody molecule, if present, may be selected having regard to the proposed function of the antibody molecule, and in particular the effector functions which may be required. For example, the constant region domains may be human IgA, IgD, IgE, IgG or IgM domains. In particular, human IgG constant region domains may be used, especially of the IgG1 and IgG3 isotypes when the antibody molecule is intended for therapeutic uses and antibody effector functions are required. Alternatively, IgG2 and IgG4 isotypes may be used when the antibody molecule is intended for therapeutic purposes and antibody effector functions are not required.

An antibody used according to the invention may be prepared, expressed, created or isolated by recombinant means, such as (a) antibodies isolated from an animal (e.g., a mouse) that is transgenic or transchromosomal for the immunoglobulin genes of interest or a hybridoma prepared therefrom, (b) antibodies isolated from a host cell transformed to express the antibody of interest, e.g., from a transfectoma, (c) antibodies isolated from a recombinant, combinatorial antibody library, and (d) antibodies prepared, expressed, created or isolated by any other means that involve splicing of immunoglobulin gene sequences to other DNA sequences.

An antibody used according to the invention may be a human antibody or a humanised antibody. The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies described herein may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

Such a human antibody may be a human monoclonal antibody. Such a human monoclonal antibody may be produced by a hybridoma that includes a B cell obtained from a transgenic nonhuman animal, e.g., a transgenic mouse, having a genome comprising a human heavy chain transgene and a light chain transgene fused to an immortalized cell.

Human antibodies may be prepared by in vitro immunisation of human lymphocytes followed by transformation of the lymphocytes with Epstein-Barr virus.

The term “human antibody derivatives” refers to any modified form of the human antibody, e.g., a conjugate of the antibody and another agent or antibody.

The term “humanized antibody” is intended to refer to CDR-grafted antibody molecules in which CDR sequences derived from the germline of another mammalian species, such as a mouse, have been grafted onto human framework sequences. Additional framework region modifications may be made within the human framework sequences.

As used herein, the term ‘CDR-grafted antibody molecule’ refers to an antibody molecule wherein the heavy and/or light chain contains one or more CDRs (including, if desired, one or more modified CDRs) from a donor antibody (e.g. a murine or rat monoclonal antibody) grafted into a heavy and/or light chain variable region framework of an acceptor antibody (e.g. a human antibody). For a review, see Vaughan et al, Nature Biotechnology, 16, 535-539, 1998. In one embodiment rather than the entire CDR being transferred, only one or more of the specificity determining residues from any one of the CDRs described herein above are transferred to the human antibody framework (see for example, Kashmiri et al., 2005, Methods, 36, 25-34). In one embodiment only the specificity determining residues from one or more of the CDRs described herein above are transferred to the human antibody framework. In another embodiment only the specificity determining residues from each of the CDRs described herein above are transferred to the human antibody framework.

When the CDRs or specificity determining residues are grafted, any appropriate acceptor variable region framework sequence may be used having regard to the class/type of the donor antibody from which the CDRs are derived, including mouse, primate and human framework regions. Suitably, the CDR-grafted antibody according to the present invention has a variable domain comprising human acceptor framework regions as well as one or more of the CDRs or specificity determining residues described above. Thus, provided in one embodiment is a neutralising CDR-grafted antibody wherein the variable domain comprises human acceptor framework regions and non-human donor CDRs.

Examples of human frameworks which can be used in the present invention are KOL, NEWM, REI, EU, TUR, TEI, LAY and POM (Kabat et al., supra). For example, KOL and NEWM can be used for the heavy chain, REI can be used for the light chain and EU, LAY and POM can be used for both the heavy chain and the light chain. Alternatively, human germline sequences may be used; these are available for example at: http://www.vbase2.org/ (see Retter et al, Nucl. Acids Res. (2005) 33 (supplement 1), D671-D674).

In a CDR-grafted antibody described herein, the acceptor heavy and light chains do not necessarily need to be derived from the same antibody and may, if desired, comprise composite chains having framework regions derived from different chains.

Also, in a CDR-grafted antibody described herein, the framework regions need not have exactly the same sequence as those of the acceptor antibody. For instance, unusual residues may be changed to more frequently occurring residues for that acceptor chain class or type. Alternatively, selected residues in the acceptor framework regions may be changed so that they correspond to the residue found at the same position in the donor antibody (see Reichmann et al., 1998, Nature, 332, 323-324). Such changes should be kept to the minimum necessary to recover the affinity of the donor antibody. A protocol for selecting residues in the acceptor framework regions which may need to be changed is set forth in WO 91/09967.

It will also be understood by one skilled in the art that antibodies may undergo a variety of posttranslational modifications. The type and extent of these modifications often depends on the host cell line used to express the antibody as well as the culture conditions. Such modifications may include variations in glycosylation, methionine oxidation, diketopiperazine formation, aspartate isomerization and asparagine deamidation. A frequent modification is the loss of a carboxy-terminal basic residue (such as lysine or arginine) due to the action of carboxypeptidases (as described in Harris, R J. Journal of Chromatography 705:129-134, 1995).

In one embodiment the antibody heavy chain comprises a CH1 domain and the antibody light chain comprises a CL domain, either kappa or lambda.

Biological molecules, such as antibodies or fragments, contain acidic and/or basic functional groups, thereby giving the molecule a net positive or negative charge. The amount of overall “observed” charge will depend on the absolute amino acid sequence of the entity, the local environment of the charged groups in the 3D structure and the environmental conditions of the molecule. The isoelectric point (pI) is the pH at which a particular molecule or surface carries no net electrical charge. In one embodiment the antibody or fragment according to the present disclosure has an isoelectric point (pI) of at least 7. In one embodiment the antibody or fragment has an isoelectric point of at least 8, such as 8.5, 8.6, 8.7, 8.8 or 9. In one embodiment the pI of the antibody is 8. Programs such as ** ExPASY http://www.expasy.ch/tools/pi tool.html (see Walker, The Proteomics Protocols Handbook, Humana Press (2005), 571-607) may be used to predict the isoelectric point of the antibody or fragment.

In order to characterise preferred Gremlin-1 epitopes, the inventors have crystallised human Gremlin-1 alone, and in complex with an antibody termed Ab 7326 (Fab fragments). Crystallisation of Gremlin-1 has allowed putative residues in the BMP binding site to be determined. Furthermore, crystallisation with Ab 7326, which is an allosteric inhibitory antibody, has allowed residues in the antibody epitope to be determined. Antibodies binding this epitope have particular potential as therapeutic agents in the treatment of diseases associated with Gremlin-1.

The preferred Ab 7326 antibody described herein has been identified to bind the following residues of Gremlin-1: Ile10 (131), Lys126 (147), Lys127 (148), Phe128 (149), Thr129 (150), Thr130 (151), Arg148 (169), Lys153 (174) and Gln154 (175), where Lys126 (147), Lys127 (148), Phe128 (149), Thr129 (150), Thr130 (151), Arg148 (169), Lys153 (174) and Gln154 (175) are present on one Gremlin-1 monomer and Ile110 (131) is present on the second Gremlin-1 monomer. Numbering not in brackets is based on the structural file and (which matches the numbering of mouse Gremlin-2 based on structural alignment). The numbers in brackets represent the residues based on the UniProt entry 060565 of SEQ ID NO: 1. These epitope residues were identified using NCONT analysis at 4 Å from the Gremlin-1-Ab 7326 Fab complex.

Antibodies described herein may therefore bind to an epitope which comprises at least one residue selected from Ile131, Lys147, Lys148, Phe149, Thr150, Thr151, Arg169, Lys174 and Gln175 (with residue numbering based on SEQ ID NO: 1). Antibodies described herein may bind an epitope which comprises 2, 3, 4, 5, 6, 7, 8 or all 9 of these residues (preferably at least 5 residues).

Antibodies described herein may also recognise an epitope where Ile131 is present on a different Gremlin-1 monomer to the other residues.

Although these residues are provided for a particular sequence of human Gremlin-1, the skilled person could readibly extrapolate the positions of these residues to to other corresponding Gremlin sequences (e.g. mouse) using routine techniques. Antibodies binding to epitopes comprising the corresponding residues within these other Gremlin sequences are therefore also provided by the invention.

To screen for antibodies that bind to a particular epitope, a routine cross-blocking assay such as that described in Antibodies, Harlow and Lane (Cold Spring Harbor Press, Cold Spring Harb., NY) can be performed. Other methods include alanine scanning mutants, peptide blots (Reineke (2004) Methods Mol Biol 248:443-63), or peptide cleavage analysis. In addition, methods such as epitope excision, epitope extraction and chemical modification of antigens can be employed (Tomer (2000) Protein Science 9: 487-496). Such methods are well known in the art.

Antibody epitopes may also be determined by x-ray crystallography analysis. Antibodies of the present disclosure may therefore be assessed through x-ray crystallogray analysis of the antibody bound to Gremlin-1. Epitopes may, in particular, be identified in this way by determining residues on Gremlin-1 within 4 Å of an antibody paratope residue.

An antibody as described herein may thus bind to an epitope on Gremlin-1 comprising at least one residue selected from Trp93, Phe117, Tyr119, Phe125, Tyr126 and Phe138, wherein the residue numbering is according to SEQ ID NO: 1. Further described herein is an antibody which binds an epitope comprising all of Trp93, Phe117, Tyr119, Phe125, Tyr126 and Phe138. Additionally described is an antibody which binds an epitope which comprises the following residues: Ile131, Lys147, Lys148, Phe149, Thr150, Thr151, Arg169, Lys174 and Gln175. Preferably, Lys147, Lys148, Phe149, Thr150, Thr151, Arg169, Lys174 and Gln175 are located on one monomer of Gremlin-1 and Ile131 is located on the other monomer of Gremlin-1 (Gremlin-1 dimers bind to BMP dimers).

An antibody may bind an above Gremlin-1 residue if the antibody paratope is within 4 Å of the Gremlin-1 residue as determined by x-ray crystallography.

Antibodies which bind to an epitope disclosed herein may comprise at least one, at least two or all three heavy chain CDR sequences of SEQ ID NOS: 4 to 6 (HCDR1/HCDR2/HCDR3 respectively). These are the HCDR1/HCDR2/HCDR3 sequences of the Ab 7326 antibody of the Examples as determined using Kabat methodology.

The Kabat and Chothia methods for determining CDR sequences are well known in the art (as well as other techniques). CDR sequences may be determined using any appropriate method and in the present invention, whilst Kabat is typically employed, other techniques could be used as well. In the present instance, SEQ ID NO: 3 presents the Ab 7326 HCDR1 sequence as determined using a combined Chothia & Kabat definition.

Antibodies used according to the invention may comprise at least one, at least two or all three light chain CDR sequences of SEQ ID NOS: 7 to 9 (LCDR1/LCDR2/LCDR3 respectively). These are the LCDR1/LCDR2/LCDR3 sequences of Ab 7326 using Kabat methodology.

The antibody preferably comprises at least a HCDR3 sequence of SEQ ID NO: 6.

Typically, the antibody comprises at least one heavy chain CDR sequence selected from SEQ ID NOS: 4 to 6 and at least one light chain CDR sequence selected from SEQ ID NOS 7 to 9. The antibody may comprise at least two heavy chain CDR sequences selected from SEQ ID NOS: 4 to 6 and at least two light chain CDR sequences selected from SEQ ID NOS: 7 to 9. The antibody typically comprises all three heavy chain CDR sequences of SEQ ID NOS: 4 to 6 (HCDR1/HCDR2/HCDR3 respectively) and all three light chain CDR sequences SEQ ID NOS: 7 to 9 (LCDR1/LCDR2/LCDR3 respectively). The antibodies may be chimeric, human or humanised antibodies.

The antibody may comprise a heavy chain variable region (HCVR) sequence of SEQ ID NO: 10 or 12 (the HCVR of Ab 7326 variants 1 and 2). The antibody may comprise a light chain variable region (LCVR) sequence of SEQ ID NO: 11 or 13 (the LCVR of Ab 7326 variants 1 and 2). The antibody preferably comprises the heavy chain variable region sequence of SEQ ID NO: 10 or 12 and the light chain variable region sequence of SEQ ID NO: 11 or 13 (especially HCVR/LVCR pairs of SEQ ID NOs: 10/11 or 12/13).

The antibody may comprise a heavy chain (H-chain) sequence of
SEQ ID NO: 14 mouse full length IgG1 heavy chain variant 1, or
SEQ ID NO: 28 mouse full length IgG1 heavy chain variant 2, or
SEQ ID NO: 30 human full length IgG1 heavy chain variant 1, or
SEQ ID NO: 16 human full length IgG1 heavy chain variant 2, or
SEQ ID NO: 22 human full length IgG4P heavy chain variant 1, or
SEQ ID NO: 34 human full-length IgG4P heavy chain variant 2, or
SEQ ID NO: 18 Fab heavy chain variant 1, or
SEQ ID NO: 32 Fab heavy chain variant 2.

The antibody may comprise a light chain (L-chain) sequence of
SEQ ID NO: 15 mouse full length IgG1 light chain variant 1, or
SEQ ID NO: 29 mouse full length IgG1 light chain variant 2, or
SEQ ID NO: 31 human full length IgG1 light chain variant 1, or
SEQ ID NO: 17 human full length IgG1 light chain variant 2, or
SEQ ID NO: 23 human full length IgG4P light chain variant 1, or
SEQ ID NO: 35 human full-length IgG4P light chain variant 2, or
SEQ ID NO: 19 Fab light chain variant 1, or
SEQ ID NO: 33 Fab light chain variant 2.

In one example, the antibody comprises a heavy chain/light chain sequence pair of
SEQ ID NOs: 14/15 mouse full length IgG1 variant 1, or
SEQ ID NOs: 28/29 mouse full length IgG1 variant 2, or
SEQ ID NOs: 30/31 human full length IgG1 variant 1, or
SEQ ID NOs: 16/17 human full length IgG1 variant 2, or
SEQ ID NOs: 22/23 human full length IgG4P variant 1, or
SEQ ID NOs: 34/35 human full-length IgG4P variant 2, or
SEQ ID NOs: 18/19 Fab light chain variant 1, or
SEQ ID NOs: 32/33 Fab light chain variant 2.

The variant forms of corresponding sequences may be interchanged. For example, the antibody may comprise a heavy chain/light chain sequence pair of
SEQ ID NOs: 14/29 mouse full length IgG1 heavy chain variant 1/light chain variant 2, or
SEQ ID NOs: 28/15 mouse full length IgG1 heavy chain variant 2/light chain variant 1, or
SEQ ID NOs: 30/17 human full length IgG1 heavy chain variant 1/light chain variant 2, or
SEQ ID NOs: 16/31 human full length IgG1 heavy chain variant 2/light chain variant 1, or
SEQ ID NOs: 22/35 human full length IgG4P heavy chain variant 1/light chain variant 2, or
SEQ ID NOs: 34/23 human full-length IgG4P heavy chain variant 2/light chain variant 1, or
SEQ ID NOs: 18/33 Fab heavy chain variant 1/light chain variant 2, or
SEQ ID NOs: 32/19 Fab heavy chain variant 2/light chain variant 1.

The antibodies may be chimeric, human or humanised antibodies.

The antibody may alternatively be or may comprise a variant of one of the specific sequences recited above. The following description of antibody variants is also applicable to selection of GREM1 polypeptide variants as described above.

For example, a variant may be a substitution, deletion or addition variant of any of the above amino acid sequences.

A variant antibody may comprise 1, 2, 3, 4, 5, up to 10, up to 20 or more (typically up to a maximum of 50) amino acid substitutions and/or deletions from the specific sequences discussed above. “Deletion” variants may comprise the deletion of individual amino acids, deletion of small groups of amino acids such as 2, 3, 4 or 5 amino acids, or deletion of larger amino acid regions, such as the deletion of specific amino acid domains or other features. “Substitution” variants typically involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Some properties of the 20 main amino acids which can be used to select suitable substituents are as follows:

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

“Derivatives” or “variants” generally include those in which instead of the naturally occurring amino acid the amino acid which appears in the sequence is a structural analog thereof. Amino acids used in the sequences may also be derivatized or modified, e.g. labelled, providing the function of the antibody is not significantly adversely affected.

Derivatives and variants as described above may be prepared during synthesis of the antibody or by post-production modification, or when the antibody is in recombinant form using the known techniques of site-directed mutagenesis, random mutagenesis, or enzymatic cleavage and/or ligation of nucleic acids.

Variant antibodies may have an amino acid sequence which has more than about 60%, or more than about 70%, e.g. 75 or 80%, typically more than about 85%, e.g. more than about 90 or 95% amino acid identity to the amino acid sequences disclosed herein (particularly the HCVR/LCVR sequences and the H- and L-chain sequences). Furthermore, the antibody may be a variant which has more than about 60%, or more than about 70%, e.g. 75 or 80%, typically more than about 85%, e.g. more than about 90 or 95% amino acid identity to the HCVR/LCVR sequences and the H- and L-chain sequences disclosed herein, whilst retaining the exact CDRs disclosed for these sequences. Variants may retain at least about 90%, 91%, 92%, 93%, 94%, 95%, 96%, 97%, 98% or 99% identity to the HCVR/LCVR sequences and to the H- and L-chain sequences disclosed herein (in some circumstances whilst retaining the exact CDRs).

Variants typically retain about 60%—about 99% identity, about 80%—about 99% identity, about 90%—about 99% identity or about 95%—about 99% identity. This level of amino acid identity may be seen across the full length of the relevant SEQ ID NO sequence or over a part of the sequence, such as across about 20, 30, 50, 75, 100, 150, 200 or more amino acids, depending on the size of the full length polypeptide.

In connection with amino acid sequences, “sequence identity” refers to sequences which have the stated value when assessed using ClustalW (Thompson et al., 1994, supra) with the following parameters:

Pairwise alignment parameters —Method: accurate, Matrix: PAM, Gap open penalty: 10.00, Gap extension penalty: 0.10;

Multiple alignment parameters —Matrix: PAM, Gap open penalty: 10.00, % identity for delay: 30, Penalize end gaps: on, Gap separation distance: 0, Negative matrix: no, Gap extension penalty: 0.20, Residue-specific gap penalties: on, Hydrophilic gap penalties: on, Hydrophilic residues: GPSNDQEKR. Sequence identity at a particular residue is intended to include identical residues which have simply been derivatized.

Antibodies having specific sequences and variants which maintain the function or activity of these chains are therefore provided.

Antibodies may compete for binding to Gremlin-1 with, or bind to the same epitope as, those defined above in terms of H-chain/L-chain, HCVR/LCVR or CDR sequences. In particular, an antibody may compete for binding to Gremlin-1 with, or bind to the same epitope as, an antibody which comprises a HCDR1/HCDR2/HCDR3/LCDR1/LCDR2/LCDR3 sequence combination of SEQ ID NOs: 4/5/6/7/8/9. An antibody may compete for binding to Gremlin-1 with, or bind to the same epitope as, an antibody which comprises a HCVR and LCVR sequence pair of SEQ ID NOs: 10/11 or 12/13 or full length chains of SEQ ID Nos: 14/15 or 16/17.

The term “epitope” is a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

One can easily determine whether an antibody binds to the same epitope as, or competes for binding with, a reference antibody by using routine methods known in the art. For example, to determine if a test antibody binds to the same epitope as a reference antibody of the invention, the reference antibody is allowed to bind to a protein or peptide under saturating conditions. Next, the ability of a test antibody to bind to the protein or peptide is assessed. If the test antibody is able to bind to the protein or peptide following saturation binding with the reference antibody, it can be concluded that the test antibody binds to a different epitope than the reference antibody. On the other hand, if the test antibody is not able to bind to protein or peptide following saturation binding with the reference antibody, then the test antibody may bind to the same epitope as the epitope bound by the reference antibody of the invention.

To determine if an antibody competes for binding with a reference antibody, the above-described binding methodology is performed in two orientations. In a first orientation, the reference antibody is allowed to bind to a protein/peptide under saturating conditions followed by assessment of binding of the test antibody to the protein/peptide molecule. In a second orientation, the test antibody is allowed to bind to the protein/peptide under saturating conditions followed by assessment of binding of the reference antibody to the protein/peptide. If, in both orientations, only the first (saturating) antibody is capable of binding to the protein/peptide, then it is concluded that the test antibody and the reference antibody compete for binding to the protein/peptide. As will be appreciated by the skilled person, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50%, 75%, 90% or even 99% as measured in a competitive binding assay (see, e.g., Junghans et al., Cancer Res, 1990:50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding.

Experiments of this sort can be performed using ELISA, RIA, surface plasmon resonance, flow cytometry or any other quantitative or qualitative antibody-binding assay available in the art.

Antibodies can be tested for binding to Gremlin-1 by, for example, standard ELISA or Western blotting. An ELISA assay can also be used to screen for hybridomas that show positive reactivity with the target protein. The binding selectivity of an antibody may also be determined by monitoring binding of the antibody to cells expressing the target protein, for example by flow cytometry. Thus, a screening method may comprise the step of identifying an antibody that is capable of binding Gremlin-1 by carrying out an ELISA or Western blot or by flow cytometry.

Antibodies may selectively (or specifically) recognise Gremlin-1. An antibody, or other compound, “selectively binds” or “selectively recognises” a protein when it binds with preferential or high affinity to the protein for which it is selective but does not substantially bind, or binds with low affinity, to other proteins. The selectivity of an antibody may be further studied by determining whether or not the antibody binds to other related proteins as discussed above or whether it discriminates between them. Antibodies used according to the invention typically recognise human Gremlin-1.

Antibodies may also have cross-reactivity for related proteins, or for human Gremlin-1 and for Gremlin-1 from other species.

By specific (or selective), it will be understood that the antibody binds to the protein of interest with no significant cross-reactivity to any other molecule. Cross-reactivity may be assessed by any suitable method described herein. Cross-reactivity of an antibody may be considered significant if the antibody binds to the other molecule at least about 5%, 10%, 15%, 20%, 25%, 30%, 35%, 40%, 45%, 50%, 55%, 60%, 65%, 70%, 75%, 80%, 85%, 90% or 100% as strongly as it binds to the protein of interest. An antibody that is specific (or selective) may bind to another molecule at less than about 90%, 85%, 80%, 75%, 70%, 65%, 60%, 55%, 50%, 45%, 40%, 35%, 30%, 25% or 20% the strength that it binds to the protein of interest. The antibody may bind to the other molecule at less than about 20%, less than about 15%, less than about 10% or less than about 5%, less than about 2% or less than about 1% the strength that it binds to the protein of interest.

Anti-gremlin antibodies have been previously described, for example WO2014/159010A1 (Regeneron) describes anti-gremlin antibodies that inhibit Gremlin-1 activity, with binding affinity K_{D} values ranging from 625 pM to 270 nM at 25° C. Ciuclan et al (2013) describe an anti-Gremlin-1 monoclonal antibody with a binding affinity K_{D}5.6×10⁻¹⁰ M.

The anti-Gremlin-1 antibodies described herein (and also in WO/2018/115017 filed 19 Dec. 2017 and WO 2019/243801 filed 18 Jun. 2019, both of which are incorporated herein by reference in its entirety) are allosteric inhibitors of Gremlin-1 activity, and bind to a novel epitope as described above, distal from the BMP binding site. The antibodies bind to Gremlin-1 with exceptionally high affinity with Kd values <100 pM. The antibodies therefore represent a significant improvement over currently available antibodies and are expected to be particularly useful for the treatment of Gremlin-1 mediated diseases.

Thus, antibodies suitable for use with the present invention may have a high affinity binding for (human) Gremlin-1. The antibody may have a dissociation constant (K_{D}) of less than <1 nM, and preferably <500 pM. In one example, the antibody has a dissociation constant (K_{D}) of less than 200 pM. In one example, the antibody has a dissociation constant (K_{D}) of less than 100 pM. A variety of methods can be used to determine the binding affinity of an antibody for its target antigen such as surface plasmon resonance assays, saturation assays, or immunoassays such as ELISA or RIA, as are well known to persons of skill in the art. An exemplary method for determining binding affinity is by surface plasmon resonance analysis on a BIAcore™ 2000 instrument (Biacore AB, Freiburg, Germany) using CM5 sensor chips, as described by Krinner et al., (2007) Mol. Immunol. February; 44 (5):916-25. (Epub 2006 May 11)).

Antibodies used according to the invention are typically inhibitory antibodies. Gremlin-1 negatively regulates BMP-2, 4 and 7, so inhibition of Gremlin-1 results in increased signalling through BMP.

Particular functional assays that may be used for screening whether an antibody is capable of inhibiting Gremlin 1 include the SMAD phosphorylation assay and the Hek Id1 reporter gene assay. Typically, an inhibitory antibody restores SMAD phosphorylation and/or restores signalling of BMP in the Hek Id1 reporter gene assay. SMAD phosphorylation may be restored to at least 80%, 90% or 100% when compared with a BMP control. In the Hek Id1 reporter gene assay, an inhibitory antibody may have an IC₅₀ of less than 10 nM, preferably less than 5 nM.

Once a suitable antibody has been identified and selected, the amino acid sequence of the antibody may be identified by methods known in the art. The genes encoding the antibody can be cloned using degenerate primers. The antibody may be recombinantly produced by routine methods.

The present disclosure also provides an isolated DNA sequence encoding the heavy and/or light chain variable regions(s) (or the full length H- and L-chains) of an antibody molecule newly described herein.

A variant polynucleotide may comprise 1, 2, 3, 4, 5, up to 10, up to 20, up to 30, up to 40, up to 50, up to 75 or more nucleic acid substitutions and/or deletions from any of the nucleic acid sequences (including GREM1 and anti-GREM1 antibody nucleic acid sequences) given in the sequence listing. Generally, a variant has 1-20, 1-50, 1-75 or 1-100 substitutions and/or deletions.

Suitable variants may be at least about 70% homologous to a polynucleotide of any one of nucleic acid sequences disclosed herein, typically at least about 80 or 90% and more suitably at least about 95%, 97% or 99% homologous thereto. Variants may retain at least about 90%, 91%, 92%, 93%, 94%, 95%, 96%, 97%, 98% or 99% identity. Variants typically retain about 60%—about 99% identity, about 80%—about 99% identity, about 90%—about 99% identity or about 95%—about 99% identity. Homology and identity at these levels is generally present at least with respect to the coding regions of the polynucleotides. Methods of measuring homology are well known in the art and it will be understood by those of skill in the art that in the present context, homology is calculated on the basis of nucleic acid identity. Such homology may exist over a region of at least about 15, at least about 30, for instance at least about 40, 60, 100, 200 or more contiguous nucleotides (depending on the length). Such homology may exist over the entire length of the unmodified polynucleotide sequence.

Methods of measuring polynucleotide homology or identity are known in the art. For example the UWGCG Package provides the BESTFIT program which can be used to calculate homology (e.g. used on its default settings) (Devereux et al (1984) Nucleic Acids Research 12, p387-395).

The PILEUP and BLAST algorithms can also be used to calculate homology or line up sequences (typically on their default settings), for example as described in Altschul S. F. (1993) J Mol Evol 36:290-300; Altschul, S, F et al (1990) J Mol Biol 215:403-10.

Software for performing BLAST analysis is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89:10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90:5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, typically less than about 0.1, suitably less than about 0.01, and most suitably less than about 0.001. For example, the smallest sum probability may be in the range of about 1—about 0.001, often about 0.01—about 0.001.

The homologue may differ from a sequence in the relevant polynucleotide by less than about 3, 5, 10, 15, 20 or more mutations (each of which may be a substitution, deletion or insertion). For example, the homologue may differ by 3-50 mutations, often 3-20 mutations. These mutations may be measured over a region of at least 30, for instance at least about 40, 60 or 100 or more contiguous nucleotides of the homologue.

In one embodiment, a variant sequence may vary from the specific sequences given in the sequence listing by virtue of the redundancy in the genetic code. The DNA code has 4 primary nucleic acid residues (A, T, C and G) and uses these to “spell” three letter codons which represent the amino acids the proteins encoded in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons. A variant polynucleotide of the invention may therefore encode the same polypeptide sequence as another polynucleotide of the invention, but may have a different nucleic acid sequence due to the use of different codons to encode the same amino acids.

The DNA sequence may comprise synthetic DNA, for instance produced by chemical processing, cDNA, genomic DNA or any combination thereof.

DNA sequences which encode an antibody molecule described herein can be obtained by methods well known to those skilled in the art. For example, DNA sequences coding for part or all of the antibody heavy and light chains may be synthesised as desired from the determined DNA sequences or on the basis of the corresponding amino acid sequences.

General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing.

### Nucleic Acid Antagonists

A polynucleotide, such as a nucleic acid, is a polymer comprising two or more nucleotides. The nucleotides can be naturally occurring or artificial. A nucleotide typically contains a nucleobase, a sugar and at least one linking group, such as a phosphate, 2′O-methyl, 2′ methoxy-ethyl, phosphoramidate, methylphosphonate or phosphorothioate group. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The nucleotide is typically a ribonucleotide or deoxyribonucleotide. The nucleotide typically contains a monophosphate, diphosphate or triphosphate. Phosphates may be attached on the 5′ or 3′ side of a nucleotide.

Nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), 5-methylcytidine monophosphate, 5-methylcytidine diphosphate, 5-methylcytidine triphosphate, 5-hydroxymethylcytidine monophosphate, 5-hydroxymethylcytidine diphosphate, 5-hydroxymethylcytidine triphosphate, cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP), 5-methyl-2′-deoxycytidine monophosphate, 5-methyl-2′-deoxycytidine diphosphate, 5-methyl-2′-deoxycytidine triphosphate, 5-hydroxymethyl-2′-deoxycytidine monophosphate, 5-hydroxymethyl-2′-deoxycytidine diphosphate and 5-hydroxymethyl-2′-deoxycytidine triphosphate. The nucleotides are preferably selected from AMP, TMP, GMP, UMP, dAMP, dTMP, dGMP or dCMP.

The nucleotides may contain additional modifications. In particular, suitable modified nucleotides include, but are not limited to, 2′amino pyrimidines (such as 2′-amino cytidine and 2′-amino uridine), 2′-hyrdroxyl purines (such as, 2′-fluoro pyrimidines (such as 2′-fluorocytidine and 2′fluoro uridine), hydroxyl pyrimidines (such as 5′-α-P-borano uridine), 2′-O-methyl nucleotides (such as 2′-O-methyl adenosine, 2′-O-methyl guanosine, 2′-O-methyl cytidine and 2′-O-methyl uridine), 4′-thio pyrimidines (such as 4′-thio uridine and 4′-thio cytidine) and nucleotides have modifications of the nucleobase (such as 5-pentynyl-2′-deoxy uridine, 5-(3-aminopropyl)-uridine and 1,6-diaminohexyl-N-5-carbamoylmethyl uridine).

The nucleotides in the polynucleotide may be attached to each other in any manner. The nucleotides may be linked by phosphate, 2′O-methyl, 2′ methoxy-ethyl, phosphoramidate, methylphosphonate or phosphorothioate linkages. The nucleotides are typically attached by their sugar and phosphate groups as in nucleic acids. The nucleotides may be connected via their nucleobases as in pyrimidine dimers.

The GREM1 antagonist may be a polynucleotide encoding an anti-GREM1 antibody described herein.

The polynucleotide can be a nucleic acid, such as deoxyribonucleic acid (DNA) or a ribonucleic acid (RNA). The polynucleotide may be any synthetic nucleic acid known in the art, such as peptide nucleic acid (PNA), glycerol nucleic acid (GNA), threose nucleic acid (TNA), locked nucleic acid (LNA), morpholino nucleic acid or other synthetic polymers with nucleotide side chains. The polynucleotide may be single stranded or double stranded.

The polynucleotide sequence may be cloned into any suitable expression vector. In an expression vector, the polynucleotide sequence encoding a construct is typically operably linked to a control sequence which is capable of providing for the expression of the coding sequence by the host cell. Such expression vectors can be used to express a construct.

In one embodiment, the anti-GREM1 antagonist is a polynucleotide encoding an anti-GREM1 antibody described herein. The polynucleotide may be provided for use in gene therapy. The polynucleotide may be provided in any suitable vector capable of providing for expression of the anti-GREM1 antibody in vivo.

The polynucleotide encoding the anti-GREM1 antibody may be a DNA sequence. The DNA sequence may be provided in any suitable vector, e.g. an expression vector, for administration to a subject in need thereof. For example, the DNA sequence may be administered to the subject in an expression vector capable of providing for expression of the anti-GREM1 antibody in vivo. The expression vector may be a viral expression vector, such as an adeno-associated virus (AAV) vector. In one embodiment, the anti-GREM1 antagonist is a DNA sequence which encodes an anti-GREM1 antibody described herein. In one embodiment, the anti-GREM1 antagonist is a DNA sequence for use in gene therapy, wherein the DNA sequence encodes an anti-GREM1 antibody described herein. In one embodiment, the anti-GREM1 antagonist is an AAV comprising a DNA sequence which encodes an anti-GREM1 antibody described herein. In one embodiment, the anti-GREM1 antagonist is an AAV for use in gene therapy, wherein the AAV comprises a DNA sequence which encodes an anti-GREM1 antibody described herein.

The polynucleotide encoding the anti-GREM1 antibody may be an RNA sequence. The RNA sequence may be administered to a subject in need thereof in any suitable vector. The RNA sequence may be a messenger RNA (mRNA) sequence. The mRNA sequence may be administered to a subject in need thereof in a stabilised form. For example the mRNA sequence may be provided in a lipid nanoparticle (LNP) composition. The LNP composition may comprise any suitable LNPs capable of encapsulating the mRNA sequence to provide for increased stability of said mRNA sequence. Thus, in one embodiment, the anti-GREM1 antagonist is a stabilised mRNA sequence encoding an anti-GREM1 antibody described herein. In one embodiment, the anti-GREM1 antagonist is a stabilised mRNA sequence for use in gene therapy, wherein the mRNA sequence encodes an anti-GREM1 antibody described herein. In one embodiment, the anti-GREM1 antagonist is a LNP composition which comprises an mRNA encoding an anti-GREM1 antibody described herein. In one embodiment, the anti-GREM1 antagonist is an LNP composition for use in gene therapy, wherein the LNP composition comprises an mRNA encoding an anti-GREM1 antibody described herein.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Multiple copies of the same or different polynucleotide may be introduced into the vector.

The expression vector may then be introduced into a suitable host cell. Thus, a construct can be produced by inserting a polynucleotide sequence encoding a construct into an expression vector, introducing the vector into a compatible bacterial host cell, and growing the host cell under conditions which bring about expression of the polynucleotide sequence.

A GREM1 antagonist which is nucleic acid-based may reduce expression of GREM1. Antisense and RNA interference (RNAi) technology for knocking down protein expression are well known in the art and standard methods can be employed to knock down expression of a molecule of interest. Both antisense and siRNA technology interfere with mRNA. Antisense oligonucleotides interfere with mRNA by binding to (hybridising with) a section of the mRNA. The antisense oligonucleotide is therefore designed to be complementary to the mRNA (although the oligonucleotide does not have to be 100% complementary as discussed below). In other words, the antisense oligonucleotide may be a section of the cDNA. Again, the oligonucleotide sequence may not be 100% identical to the cDNA sequence. This is also discussed below. RNAi involves the use of double-stranded RNA, such small interfering RNA (siRNA) or small hairpin RNA (shRNA), which can bind to the mRNA and inhibit protein expression.

Accordingly, the antagonist may be a oligonucleotide which specifically hybridises to an mRNA encoding GREM1, such as the encoding sequence of SEQ ID NO: 36 or SEQ ID NO: 37 or a variant thereof. An oligonucleotide “specifically hybridises” to a target sequence when it hybridises with preferential or high affinity to the target sequence but does not substantially hybridise, does not hybridise or hybridises with only low affinity to other sequences. More preferably, the oligonucleotide hybridises to the target sequence with a Tm that is at least 5° C., at least at least 10° C., at least 20° C., at least 30° C. or at least 40° C., greater than its Tm for other nucleic acids. Conditions that permit the hybridisation are well-known in the art (for example, Sambrook et al., 2001, Molecular Cloning: a laboratory manual, 3rd edition, Cold Spring Harbour Laboratory Press; and Current Protocols in Molecular Biology, Chapter 2, Ausubel et al., Eds., Greene Publishing and Wiley-Interscience, New York (1995)). The hybridisation conditions may be stringent conditions as described in the art.

Oligonucleotides are short nucleotide polymers which typically have 50 or fewer nucleotides, such 40 or fewer, 30 or fewer, 22 or fewer, 21 or fewer, 20 or fewer, 10 or fewer or 5 or fewer nucleotides. The oligonucleotide used may be 20 to 25 nucleotides in length, more preferably 21 or 22 nucleotides in length. The nucleotides can be naturally occurring or artificial. The nucleotides can be any of those described above.

The GREM1 antagonist may be an antibody that binds to GREM1, typically specifically binding GREM1. An antibody “specifically binds” to a protein when it binds with preferential or high affinity to that protein but does not substantially bind, does not bind or binds with only low affinity to other proteins. For instance, an antibody “specifically binds” a target molecule when it binds with preferential or high affinity to that target but does not substantially bind, does not bind or binds with only low affinity to other human proteins.

An antibody binds with preferential or high affinity if it binds with a Kd of 1×10-7 M or less, more preferably 5×10-8 M or less, more preferably 1×10-8 M or less or more preferably 5×10-9 M or less. An antibody binds with low affinity if it binds with a Kd of 1×10-6 M or more, more preferably 1×10-5 M or more, more preferably 1×10-4 M or more, more preferably 1×10-3 M or more, even more preferably 1×10-2 M or more.

The antibody may be, for example, a monoclonal antibody, a polyclonal antibody, a single chain antibody, a chimeric antibody, a bispecific antibody, a CDR-grafted antibody or a humanized antibody. The antibody may be an intact immunoglobulin molecule or a fragment thereof such as a Fab, F(ab′)₂ or Fv fragment.

### Patient

Any patient may be treated in accordance with the invention. The patient is typically human. However, the patient may be another mammalian animal, such as a commercially farmed animal, such as a horse, a cow, a sheep, a fish, a chicken or a pig, a laboratory animal, such as a mouse or a rat, or a pet, such as a guinea pig, a hamster, a rabbit, a cat or a dog.

### Pharmaceutical Compositions, Dosages and Dosage Regimes

A GREM1 antagonist for use in a method of the invention may be provided in a pharmaceutical composition. The inhibitor of Ras-Raf-MEK-ERK signalling (such as a MEK inhibitor or ERK inhibitor as described herein) for use in a method of the invention may also be provided as part of the same pharmaceutical composition or in a separate pharmaceutical composition. For example, a GREM1 antagonist for use in a method of the invention may be provided in a pharmaceutical composition together with a MEK inhibitor or ERK inhibitor as described herein, or other inhibitors of Ras-Raf-MEK-ERK signalling. The pharmaceutical composition will normally be sterile and will typically include a pharmaceutically acceptable carrier and/or adjuvant. These compositions may comprise, in addition to the therapeutically active ingredient(s), a pharmaceutically acceptable excipient, carrier, diluent, buffer, stabiliser or other materials well known to those skilled in the art. Such materials should be non-toxic and should not interfere with the efficacy of the active ingredient. The pharmaceutical carrier or diluent may be, for example, an isotonic solution.

As used herein, “pharmaceutically acceptable carrier” includes any and all solvents, dispersion media, coatings, antibacterial and antifungal agents, isotonic and absorption delaying agents, and the like that are physiologically compatible. The precise nature of the carrier or other material may depend on the route of administration, e.g. oral, intravenous, cutaneous or subcutaneous, nasal, intramuscular and intraperitoneal routes.

The carrier may be suitable for parenteral, e.g. intravenous, intramuscular, intradermal, intraocular, intraperitoneal, subcutaneous, spinal or other parenteral routes of administration, for example by injection or infusion. Alternatively, the carrier may be suitable for non-parenteral administration, such as a topical, epidermal or mucosal route of administration. The carrier may be suitable for oral administration. Depending on the route of administration, the modulator may be coated in a material to protect the compound from the action of acids and other natural conditions that may inactivate the compound. For example, solid oral forms may contain, together with the active substance, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Other oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the pharmaceutical composition is lyophilised, the lyophilised material may be reconstituted prior to administration, e.g. a suspension. Reconstitution is preferably effected in buffer.

Capsules, tablets and pills for oral administration to an individual may be provided with an enteric coating comprising, for example, Eudragit “S”, Eudragit “L”, cellulose acetate, cellulose acetate phthalate or hydroxypropylmethyl cellulose.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active substance, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

For suppositories, traditional binders and carriers may include, for example, polyalkylene glycols or triglycerides; such suppositories may be formed from mixtures containing the active ingredient in the range of 0.5% to 10%, preferably 1% to 2%.

Polynucleotide or oligonucleotide inhibitors maybe naked nucleotide sequences or be in combination with cationic lipids, polymers or targeting systems. They may be delivered by any available technique. For example, the polynucleotide or oligonucleotide may be introduced by needle injection, preferably intradermally, subcutaneously or intramuscularly. Alternatively, the polynucleotide or oligonucleotide may be delivered directly across the skin using a delivery device such as particle-mediated gene delivery. The polynucleotide or oligonucleotide may be administered topically to the skin, or to mucosal surfaces for example by intranasal, oral, or intrarectal administration.

Uptake of polynucleotide or oligonucleotide constructs may be enhanced by several known transfection techniques, for example those including the use of transfection agents. Examples of these agents include cationic agents, for example, calcium phosphate and DEAE-Dextran and lipofectants, for example, lipofectam and transfectam. The dosage of the polynucleotide or oligonucleotide to be administered can be altered.

The pharmaceutical compositions of the invention may include one or more pharmaceutically acceptable salts. A “pharmaceutically acceptable salt” refers to a salt that retains the desired biological activity of the parent compound and does not impart any undesired toxicological effects. Examples of such salts include acid addition salts and base addition salts.

Pharmaceutically acceptable carriers comprise aqueous carriers or diluents. Examples of suitable aqueous carriers that may be employed in the pharmaceutical compositions of the invention include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. In many cases, it will be desirable to include isotonic agents, for example, sugars, polyalcohols such as mannitol, sorbitol, or sodium chloride in the composition.

Therapeutic compositions typically must be sterile and stable under the conditions of manufacture and storage. The composition can be formulated as a solution, microemulsion, liposome, or other ordered structure suitable to high drug concentration.

Pharmaceutical compositions of the invention may comprise additional active ingredients.

Also within the scope of the present disclosure are kits comprising the combination therapies described herein and instructions for use. The kit may further contain one or more additional reagents, such as an additional therapeutic or prophylactic agent as discussed herein.

The antagonists described herein or formulations or compositions thereof may be administered for prophylactic and/or therapeutic treatments.

In therapeutic applications, compounds are administered to a subject already suffering from a disorder or condition as described above, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as a “therapeutically effective amount”.

In prophylactic applications, formulations are administered to a subject at risk of a disorder or condition as described above, in an amount sufficient to prevent or reduce the subsequent effects of the condition or one or more of its symptoms. An amount adequate to accomplish this is defined as a “prophylactically effective amount”. Effective amounts for each purpose will depend on the severity of the disease or injury as well as the weight and general state of the subject.

A subject for administration may be a human or non-human animal. The term “non-human animal” includes all vertebrates, e.g., mammals and non-mammals, such as non-human primates, sheep, dogs, cats, horses, cows, chickens, amphibians, reptiles, etc. Administration to humans is typical.

An antagonist, inhibitor of Ras-Raf-MEK-ERK signalling (e.g. a MEK or ERK inhibitor), or pharmaceutical composition of the invention may be administered via one or more routes of administration using one or more of a variety of methods known in the art. As will be appreciated by the skilled artisan, the route and/or mode of administration will vary depending upon the desired results. Examples of routes of administration for compounds or pharmaceutical compositions of the invention include intravenous, intramuscular, intradermal, intraocular, intraperitoneal, subcutaneous, spinal or other parenteral routes of administration, for example by injection or infusion. The phrase “parenteral administration” as used herein means modes of administration other than enteral and topical administration, usually by injection. Alternatively, antibody/modulatory agent or pharmaceutical composition of the invention can be administered via a non-parenteral route, such as a topical, epidermal or mucosal route of administration. The antibody/modulatory agent or pharmaceutical composition of the invention may be for oral administration.

A suitable dosage of an antibody/modulatory agent or pharmaceutical composition of the invention may be determined by a skilled medical practitioner. Actual dosage levels of the active ingredients in the pharmaceutical compositions of the present invention may be varied so as to obtain an amount of the active ingredient that is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A suitable dose may be, for example, in the range of from about 0.01 μg/kg to about 1000 mg/kg body weight, typically from about 0.1 μg/kg to about 100 mg/kg body weight, of the patient to be treated dependent on the conditions mentioned above. For example, a suitable dosage may be from about 1 μg/kg to about 10 mg/kg body weight per day or from about 10 g/kg to about 5 mg/kg body weight per day.

Dosage regimens may be adjusted to provide the optimum desired response (e.g., a therapeutic response). For example, a single dose may be administered, several divided doses may be administered over time or the dose may be proportionally reduced or increased as indicated by the exigencies of the therapeutic situation. The dose may be provided as multiple doses, for example taken at regular intervals, for example 2, 3 or 4 doses administered hourly. Multiple doses may be administered via the same or different routes and to the same or different locations. Alternatively, doses can be via a sustained release formulation, in which case less frequent administration is required. Dosage and frequency may vary depending on the half-life of the antagonist in the patient and the duration of treatment desired.

Typically polynucleotide or oligonucleotide inhibitors are administered in the range of 1 pg to 1 mg, preferably to 1 pg to 10 μg nucleic acid for particle mediated delivery and μg to 1 mg for other routes.

Dosage unit form as used herein refers to physically discrete units suited as unitary dosages for the subjects to be treated; each unit contains a predetermined quantity of active compound calculated to produce the desired therapeutic effect in association with the required pharmaceutical carrier.

As mentioned above, modulators/antibodies or pharmaceutical compositions of the invention may be co-administered with one or other more other therapeutic agents.

Examples of the techniques and protocols mentioned above can be found in Remington's Pharmaceutical Sciences, 20th Edition, 2000, pub. Lippincott, Williams & Wilkins.

Combined administration of two or more agents may be achieved in a number of different ways. Both may be administered together in a single composition, or they may be administered in separate compositions as part of a combined therapy. For example, the one may be administered before or separately, after or sequential, or concurrently or simultaneously with the other. For example, the anti-GREM1 antagonist may be administered before or separately, after or sequential, or concurrently or simultaneously with the inhibitor of Ras-Raf-MERK-ERK signalling. For example, the anti-GREM1 antagonist may be administered before or separately, after or sequential, or concurrently or simultaneously with the MEK inhibitor as described herein. In another aspect of the present invention, the anti-GREM1 antagonist may be administered before or separately, after or sequential, or concurrently or simultaneously with the ERK inhibitor as described herein.

### Compositions and Kits

Additionally provided is a composition or kit comprising an anti-GREM1 antagonist and an inhibitor of Ras-Raf-MEK-ERK signalling. An anti-GREM1 antagonist may be any anti-GREM1 antagonist as described herein. An inhibitor of Ras-Raf-MEK-ERK signalling may be any inhibitor of Ras-Raf-MEK-ERK signalling as described herein. For example, the present invention provides a composition or kit comprising an anti-GREM1 antagonist and a MEK inhibitor. In another aspect, the present invention provides a composition or kit comprising an anti-GREM1 antagonist and an ERK inhibitor. The composition or kit may be suitable for the treatment of pancreatic cancer. In a preferred embodiment, the MEK inhibitor is selumetinib (AZD6244), trametinib (GSK-1120212) or WX-554 (UCB-554), particularly as part of a composition or kit for treatment of pancreatic cancer. A preferred combination comprises an anti-GREM1 antagonist and selumetinib (AZD6244), trametinib (GSK-1120212) or WX-554 (UCB-554). In another preferred embodiment, the ERK inhibitor is ulixertinib.

A bispecific antibody combining an anti-GREM1 specificity and one of the other above specificities may be provided in a composition or kit as described herein. The anti-GREM1 antagonist in any of the above compositions and kits may preferably be an anti-GREM1 antibody.

### Additional Therapeutic Combinations

A combination therapy of the invention as described above may be used/administered in combination with a further therapeutic composition for treatment, for example as adjunct therapy. The other therapeutic compositions or treatments may for example be one or more of those discussed herein, and may be administered either simultaneously or sequentially with the composition of the invention.

GREM1 antagonists have particular utility in combination treatments, since they may be used to sensitise a cancer or tumour to a further anti-cancer agent, such as radiotherapy or surgery. The cancer may be resistant to the other anti-cancer agent or cancer therapy in the absence of the GREM1 antagonist.

Thus, an anti-GREM1 antagonist in combination with an inhibitor of Ras-Raf-MEK-ERK signalling may further be used in combination with any other cancer therapy or any other therapeutic agent for a cancer. The other cancer therapy may be selected from any known therapy for the relevant cancer, such as any known therapy for pancreatic cancer. The other cancer therapy may be a radiotherapy. Suitable radiotherapy treatments are described for example in Van Cutsem (and others) Annals of Oncology, 2014. Vol 25, Issue 3. The radiotherapy may be carried out before surgery on a cancer or after surgery on a cancer. The radiotherapy may be adjuvant radiotherapy. The radiotherapy may be carried out in combination with an inhibitor of Ras-Raf-MEK-ERK signalling as described herein. For example, a combination therapy comprising a GREM1 antagonist and selumetinib (AZD6244) may be used in combination with radiotherapy. In addition, a combination therapy comprising a GREM1 antagonist and trametinib (GSK-1120212) may be used in combination with radiotherapy. Alternatively, a combination therapy comprising a GREM1 antagonist and WX-554 (UCB-554) may be used in combination with radiotherapy. A combination therapy comprising a GREM1 antagonist and any MEK inhibitor as described herein may be used in combination with radiotherapy. Alternatively, a combination therapy comprising a GREM1 antagonist and any ERK inhibitor as described herein may be used in combination with radiotherapy. For example, a combination therapy comprising a GREM1 antagonist and ulixertinib may be used in combination with radiotherapy.

The cancer may be resistant to radiotherapy when not administered together with a combination therapy comprising a GREM1 antagonist as described herein.

As part of the above aspects, the invention provides an anti-GREM1 antagonist in combination with an inhibitor of Ras-Raf-MEK-ERK signalling for use in a method of treatment and/or prevention of cancer, such as pancreatic cancer, according to the invention, wherein the method further comprises separate, sequential or simultaneous administration of an additional anti-cancer agent. In a preferred embodiment, the invention provides an anti-GREM1 antagonist in combination with one or more MEK inhibitors (such as selumetinib (AZD6244), trametinib (GSK-1120212) and/or WX-554 (UCB-554)) for use in a method of treatment and/or prevention of cancer, such as pancreatic cancer, according to the invention, wherein the method further comprises separate, sequential or simultaneous administration of an additional anti-cancer agent.

As part of the above aspects, the invention provides an anti-GREM1 antagonist in combination with one or more Ras-Raf-MEK-ERK signalling inhibitors for use in a method of treatment and/or prevention of cancer, such as pancreatic cancer. For example, the invention may provide an anti-GREM1 antagonist in combination with the MEK inhibitor selumetinib (AZD6244) for use in a method of treatment and/or prevention of cancer, such as pancreatic cancer, according to the invention, wherein the method further comprises separate, sequential or simultaneous administration of an additional MEK inhibitor, such as trametinib (GSK-1120212), cobimetinib (GDC-0973), binimetinib (MEK162), CI-1040 (PD184352), Mirdametinib (PD0325901), TAK733, refametinib (RDEA119/Bay 86-9766), RO-5126766, RO5068760, Pimasertib (AS703026), AZD8330, GDC-0623, RO-4987655, WX-554 (UCB-554), HL-085, ARRY-300, ClnQ-03, G-573, PD184161, PD318088, PD98059, U0126 or SL327.

In an alternative embodiment, the invention may provide an anti-GREM1 antagonist in combination with the MEK inhibitor trametinib (GSK-1120212) for use in a method of treatment and/or prevention of cancer, such as pancreatic cancer, according to the invention, wherein the method further comprises separate, sequential or simultaneous administration of an additional MEK inhibitor, such as selumetinib (AZD6244), cobimetinib (GDC-0973), binimetinib (MEK162), CI-1040 (PD184352), Mirdametinib (PD0325901), TAK733, refametinib (RDEA119/Bay 86-9766), RO-5126766, RO5068760, Pimasertib (AS703026), AZD8330, GDC-0623, RO-4987655, WX-554 (UCB-554), HL-085, ARRY-300, ClnQ-03, G-573, PD184161, PD318088, PD98059, U0126 or SL327.

In yet a further alternative embodiment, the invention may provide an anti-GREM1 antagonist in combination with the MEK inhibitor WX-554 (UCB-554) for use in a method of treatment and/or prevention of cancer, such as pancreatic cancer, according to the invention, wherein the method further comprises separate, sequential or simultaneous administration of an additional MEK inhibitor, such as trametinib (GSK-1120212), selumetinib (AZD6244), cobimetinib (GDC-0973), binimetinib (MEK162), CI-1040 (PD184352), Mirdametinib (PD0325901), TAK733, refametinib (RDEA119/Bay 86-9766), RO-5126766, RO5068760, Pimasertib (AS703026), AZD8330, GDC-0623, RO-4987655, HL-085, ARRY-300, ClnQ-03, G-573, PD184161, PD318088, PD98059, U0126 or SL327.

In yet a further alternative embodiment, the invention may provide an anti-GREM1 antagonist in combination with the ERK inhibitor ulixertinib for use in a method of treatment and/or prevention of cancer, such as pancreatic cancer, according to the invention, wherein the method further comprises separate, sequential or simultaneous administration of an additional ERK inhibitor, such as ulixertinib, temuterkib, JSI-1187, ravoxertinib, ASN007 or MK-8353.

The invention may additionally provide an anti-GREM1 antagonist in combination with a MEK inhibitor and further in combination with an ERK inhibitor as described herein.

### Detection and Diagnosis

Based on the correlation between stromal GREM1 and cancer coupled with the finding that genes involved in the Ras-Raf-MEK-ERK pathway are upregulated following treatment with a GREM1 antagonist, the present invention also provides for additional means for the prediction of responsiveness of a cancer to a treatment. Such means may involve measuring Ras-Raf-MEK-ERK pathway signalling, stromal expression and/or epithelial expression of GREM1 in a sample obtained from a patient.

The invention provides a method for determining whether or not a patient having or suspected of having or being at risk of developing cancer is likely to respond to a combination treatment with a GREM1 antagonist and an inhibitor of Ras-Raf-MEK-ERK signalling, which method comprises measuring stromal and/or epithelial expression of GREM1 and Ras-Raf-MEK-ERK signalling in the patient, and thereby predicting whether or not the patient is likely to respond to treatment with the combination.

The invention additionally provides a method for determining whether or not a patient having or suspected of having or being as risk of developing cancer is likely to respond to a combination treatment with an anti-GREM1 antagonist and an inhibitor of Ras-Raf-MEK-ERK signalling, which method comprises measuring Ras-Raf-MEK-ERK pathway signalling in the patient following treatment/exposure with an anti-GREM1 antagonist, and thereby predicting whether or not the patient is likely to respond to treatment with the combination. For example, upregulated/induced Ras-Raf-MEK-ERK signalling in response to treatment with an anti-GREM1 antagonist may indicate that the patient is a good candidate for a combination therapy in accordance with the invention. The invention additionally provides a method for determining whether or not a patient having or suspected of having or being as risk of developing pancreatic cancer is likely to respond to a combination treatment with an anti-GREM1 antagonist and an inhibitor of Ras-Raf-MEK-ERK signalling, which method comprises measuring Ras-Raf-MEK-ERK pathway signalling in the patient following treatment/exposure with an anti-GREM1 antagonist, and thereby predicting whether or not the patient is likely to respond to treatment with the combination. For example, upregulated Ras-Raf-MEK-ERK signalling in response to treatment with an anti-GREM1 antagonist may indicate that the patient is a good candidate for a combination therapy in accordance with the invention. The invention additionally provides a method for determining whether or not a patient having or suspected of having or being at risk of developing pancreatic cancer is likely to respond to a combination treatment with a GREM1 antagonist and an inhibitor of Ras-Raf-MEK-ERK signalling, which method comprises measuring stromal and/or epithelial expression of GREM1 in the patient and determining whether the patient has a mutation in a RAS gene or a RAF gene, and thereby predicting whether or not the patient is likely to respond to treatment with the combination.

Predicted responsiveness in an individual to a given therapy means that the individual is expected to derive benefit, or a sufficient extent of benefit, from receiving the therapy. Predicted non-responsiveness in an individual to a therapy means that the individual is not expected to derive benefit, or a sufficient extent of benefit, from receiving the therapy. The method for predicting the response may be carried out before administration of the combination treatment with a GREM1 antagonist and the inhibitor of Ras-Raf-MEK-ERK signalling. The prediction may then be taken into account when selecting or recommending a suitable treatment for the individual. Alternatively, the method may be carried out after treatment with the therapy and used to monitor and predict the individual's response to treatment. Typically the method is for predicting whether or not the individual will have a primary response to treatment with the therapy, i.e. whether or not the individual will respond when first receiving the treatment. In some cases the method is for predicting secondary non-responsiveness, i.e. whether or not an individual who initially responds to treatment will later stop responding to treatment or will respond less well to the treatment.

In some cases, an overexpression of GREM1, as compared with a reference sample or reference level, indicates that the individual will respond to therapy with a combination treatment as described herein. Dysregulation of Ras-Raf-MEK-ERK signalling, for example up-regulation of Nras, Mapk1, and/or Myc, as compared with a reference sample or reference level, may also indicate that the individual will respond to therapy with a combination treatment as described herein. Ras-Raf-MEK-ERK signalling may be measured by examining phosphorylation levels, for example, using phosphorylation site-specific antibodies, such as phospho-specific antibodies to MEK and ERK. For example, increased phosphorylation of Map2kl/Map2k2/Erk1/Erk2 in response to treatment with an anti-GREM1 antagonist of the invention, as compared with a reference sample or reference level, indicates that the individual will respond to therapy with a combination treatment as described herein. A combination therapy comprising a GREM1 antagonist in combination with an inhibitor of Ras-Raf-MEK-ERK signalling, such as a MEK inhibitor or ERK inhibitor may then be selected or recommended, and may then further be administered to the individual.

In other cases, a decreased or normal level of GREM1, as compared with a reference sample or reference level, indicates that the individual will not respond to therapy with a GREM1 antagonist. Normal or decreased Ras-Raf-MEK-ERK signalling, as compared with a reference sample or reference level, may indicate that an individual will not respond to therapy with the inhibitor of Ras-Raf-MEK-ERK signalling. A combination therapy comprising a GREM1 antagonist and an inhibitor of Ras-Raf-MEK-ERK signalling is then not administered to the individual. Further, a therapeutic treatment other than a combination therapy described herein may be selected or recommended for treatment of the individual, and may then further be administered to the individual.

In yet other cases, a mutation in a RAS gene (such as KRAS, NRAS and/or HRAS) or a RAF gene (such as ARAF, BRAF and/or CRAF) indicates that the individual will respond to therapy with a combination treatment as described herein. A combination therapy comprising a GREM1 antagonist in combination with an inhibitor of Ras-Raf-MEK-ERK signalling may then be selected or recommended, and may then further be administered to the individual.

In all aspects of the invention, an individual having cancer (e.g. pancreatic cancer) or an individual suspected of having the disease or condition and/or an individual at risk of developing the disease or condition may be selected for treatment or identified. For example, the individual may not have been formally diagnosed but may be suspected of having the disease or condition because of the presence of one or more symptoms. The individual may be considered at risk of developing cancer if they have one or more risk factors associated with cancer and/or one or more predispositions which increase their susceptibility to cancer. Risk factors in relation to pancreatic cancer can include familial pancreatitis caused by mutations in the PRSS1 gene and inherited genetic mutations such as mutation or mutations in the GREM1 encoding gene, or any other mutation affecting expression of the GREM1 gene.

The following Examples illustrate the invention.

### EXAMPLES

### Materials and Methods

Anti-gremlin-1 antibody—Ab7326 mIgG1—APP.4405.IgG.mFc

Vehicle—Phosphate Buffered Saline pH 7.4 (provided by the Beatson Institute) Selumetinib (AZD6244)—provided by the Beatson Institute (purchased from SYNthesis Pharm), in 0.5% HPMC+0.1% Tween-80.

UCB-554 MEK inhibitor (UCB1366554-000, Batch ID B10476792, provided by UCB) in 10% DMSO+90% of 1% (w/v) methyl cellulose. Dosing was 10 mg/kg in 125 μl, p.o., q2d

Vehicle in Example 5 was 125 μl of 10% DMSO+90% of 1% (w/v) methyl cellulose, p.o., q2d

### Genetically Modified Mice

LSL-Kras^{G12D/+}; Trp53^{R172H/+}; Pdx1-Cre (KPC) mice have been described previously (Hingorani et al., 2005). Mice were generated by crossing mice bearing Pdx1-Cre, and conditional LSL-KrasG12D or LSL-Trp53R172H alleles (strains 01XJ6 and 01XL9, Mouse Models of Human Cancer Consortium [MMHCC], NCI-Frederick, Frederick, MD, USA). Mice on a mixed background were bred in-house at the CRUK Beatson Institute and maintained in conventional caging with environmental enrichment, access to standard chow and water ad libitum. Genotyping was performed by Transnetyx (Cordoba, TN, USA). Mice of both sexes were recruited onto study. All animal experiments were performed under a UK Home Office licence and approved by the University of Glasgow Animal Welfare and Ethical Review Board.

### Treatments for Examples 3 and 4

Mice were monitored 3 times weekly until a diagnosis of pancreatic cancer was made by abdominal palpation and confirmed by ultrasound imaging. Mice were randomised onto treatment arms and dosed with: 30 mg/kg Ab7326 mIgG1 s.c. twice weekly; or 30 mg/kg Ab7326 mIgG1 s.c. twice weekly and 25 mg/kg Selumetinib p.o., b.i.d. Historical data of mice treated with 25 mg/kg Selumetinib p.o., b.i.d. were made available. There were >12 mice per group. Mice were monitored daily and sacrificed either at 7 days post-initiation of treatment (4 mice per group) or when reaching ethical endpoint (>8 mice per group, symptoms include abdominal distension, cachexia, intermittent hunching or reduced mobility, piloerection, mild diarrhoea, anaemia). Statistical assessment of survival of these mice from start of treatment was carried out by Kaplan-Meier and Log-Rank analysis.

### Ultrasound Imaging

The VisualSonics Vevo 3100 preclinical imaging platform (FUJIFILM VisualSonics, Toronto, Canada) was used for high-resolution ultrasound imaging to confirm tumour diagnosis and for weekly monitoring of tumour progression. Anaesthesia was induced and maintained with a mixture of isoflurane and medical air. Tumour volume was calculated weekly for each mouse and plotted longitudinally.

### Sampling

Mice were culled by Schedule 1 method, as per Institutional guidelines. Post-mortem tumour burden was assessed by gross pathology and histology. Organs were removed and fixed in 10% buffered formalin. At sacrifice, where possible, a terminal blood sample was taken via cardiac puncture and processed for serum. The majority of the tumour was fixed in 10% buffered formalin for FFPE processing, and any remainder harvested into RNAlater® (Sigma-Aldrich) for RNA prep and/or flash frozen. Fixed tissues were paraffin embedded and 5 m sections placed on poly-L-lysine slides for IHC analysis.

### Histology and Immunohistochemistry

H&E and Picrosirius red staining was carried out on formalin-fixed, paraffin-embedded tissues as described previously. Immunohistochemistry was performed using standard protocols. Briefly, formalin-fixed paraffin-embedded sections were deparaffinized and rehydrated by passage through Xylene and a graded alcohol series. Endogenous peroxidase activity was inactivated by treatment with hydrogen peroxide, after which citrate buffer antigen retrieval was performed. Sections were blocked in serum, and then incubated with primary antibody. Sections were incubated in secondary antibody for 30 minutes and staining visualized with 3,3′-diaminobenzidine tetrahydrochloride. Primary antibodies used were anti-alpha-SMA (1A4, Sigma-Aldrich) 1:20,000), and anti-podoplanin (RTD4E10, AbCam 1:250).

### Treatments for Example 5

Mice were monitored 3 times weekly until a diagnosis of pancreatic cancer was made by abdominal palpation and confirmed by ultrasound imaging. Mice were randomised onto treatment arms (n=9-11 per group) and dosed with: 125 μl vehicle, p.o., q2d; 30 mg/kg Ab7326 mIgG1 s.c. twice weekly; 25 mg/kg Selumetinib p.o., b.i.d.; 30 mg/kg Ab7326 mIgG1 s.c. twice weekly and 25 mg/kg Selumetinib p.o., b.i.d.; UCB-554 10 mg/kg in 125 μl, p.o., q2d; or 30 mg/kg Ab7326 mIgG1 s.c. twice weekly and UCB-554 10 mg/kg in 125 μl, p.o., q2d. Mice were monitored daily and sacrificed when reaching ethical endpoint.

### Statistics for Example 5

Statistical assessment of survival of these mice from start of treatment was carried out using a Cox's proportional hazards model to calculate hazard ratios. Tumour volume data at day 7 data were analysed using a linear model suitable for a randomised design, fitting Treatment Tumour volume at day 0 was used as a covariate to increase the precision of the estimates.

### Example 1: Confirmation of Grem1 mRNA Expression in Pancreatic Cancer

In order to investigate whether gremlin-1 might play a role in pancreatic cancer, Grem1 mRNA expression, as determined by RNAseq analysis, was determined in a cohort of human pancreatic ductal adenocarcinoma (PDAC) patients. This confirmed that Grem1 mRNA was expressed in human PDAC, and that high expression was significantly associated with poor prognosis (FIG. 1). The results were consistent with a published study reporting a link between Grem1 expression and poor outcome in pancreatic cancer (Yu et al., 2018).

To determine whether grem1 mRNA was expressed in the KPC genetically engineered mouse model of pancreatic cancer, gene expression was examined in KPC tumours (and other autochthonous pancreatic cancer models) and compared to control KrasG12D-expressing normal pancreatic duct epithelium. The results indicate that grem1 mRNA expression was elevated in KPC tumours (and other murine pancreatic tumours) compared with control (Table 1)

TABLE 1

| | | KPC | | All mouse | |
|---|---|---|---|---|---|
| | | tumour vs. | | PDAC vs. | |
| | | pancreatic | | pancreatic | |
| Gene | Gene | duct cells | | duct cells | |
| Symbol | Title | (fold change) | p-value | (fold change) | p-value |
| grem1 | gremlin 1 | 5.5 | 4.0 × 10⁻³ | 6.4 | 4.1 × 10⁻³ |

### Example 2: Gene Expression Changes in KPC Mouse Model on Treatment with Ab7326 mIgG1

KPC mice with confirmed pancreatic tumours were established as described in Materials and Methods. The mice were enrolled into cohorts for treatment with the anti-gremlin antibody Ab7326 mIgG1 or vehicle control as shown in Table 2.

The mice were sacrificed at ethical end-point and the primary tumor tissues harvested into RNAlater® (Sigma-Aldrich) for RNA extraction. RNA samples were interrogated using the Nanostring nCounter gene expression technology using a panel of approximately 750 probes.

TABLE 2

| | | | | Median |
|---|---|---|---|---|
| | | | | survival |
| TREATMENT | Dose | Schedule | n= | (days) |
| Ab7326 mIgG1 | 30 mg/kg s.c. | twice weekly | 3 | 26 |
| Vehicle (PBS) | na | twice weekly | 3 | 20 |

Analysis of gene expression changes in the KPC mouse model upon exposure to Ab7326 mIgG1 showed upregulation of the MAPK/ERK(MEK) pathway and its canonical members.

Canonical members of the MAPK/ERK(MEK) pathway Mapk1, Nras and Myc were upregulated (FIG. 3). Due to insufficient power (3 samples per group, i.e. Ab7326 mIgG1 exposure and control) these genes lost their statistical significance after multiple hypothesis correction (FDR <5%). However, the box and whisker plots depicting the difference in gene expression distribution of these three genes clearly showed an upregulated expression when comparing the Ab7326 mIgG1 to the control arm (FIG. 3).

The findings were corroborated by pathway enrichment analysis (FIG. 4). Downregulation of KRAS signaling was enriched in the downregulated set of genes (p=0.03), while upregulation of KRAS signaling was associated with the upregulated set of differentially expressed genes (p=0.01). While these enriched pathways did not remain statistically significant after multiple hypothesis correction, the coherent pattern across up-and down-regulated effects was striking.

In addition, proliferation signals went up (G2M, E2F and MYC targets) when KPC mice were exposed to Ab7326 mIgG1. This was in line with the downstream effect of MAPK/ERK(MEK) pathway activation. In FIG. 4 −log 10(p) score is displayed and only pathways with p<0.05 are retained. No multiple hypothesis correction was performed. However, MYC_TARGETS, E2F_TARGETS and G2M_CHECKPOINT pathways remained significant after this correction (FDR <5%).

The findings supported the potential combination of Ab7326 mIgG1 with a MEK inhibitor for the treatment of pancreatic cancer. Furthermore, Ab7326 mIgG1 exposure may render pancreatic cancers more susceptible to this targeted treatment.

### Example 3: Treatment with a Combination of Ab7326 mIgG1 and Selumetinib Results in Slower Tumour Growth Compared to Treatment with Ab7326 mIgG1 as a Single Agent

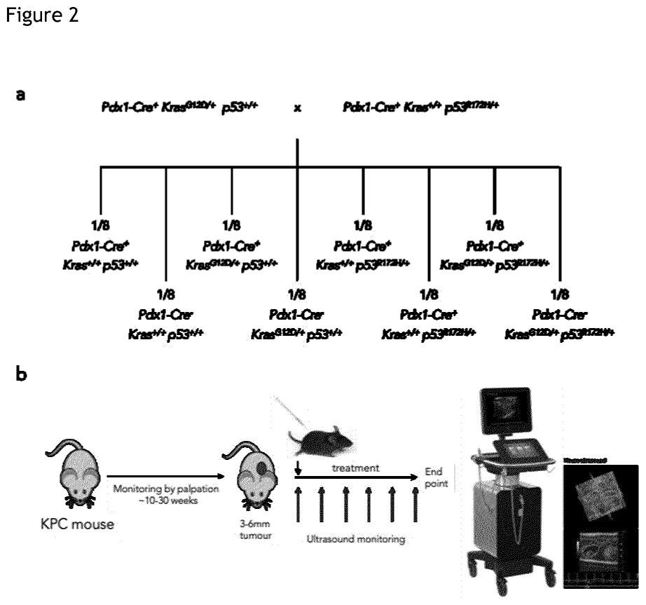

LSL-Kras^{G12D/+}; LSL-Trp53^{R172H/+}; Pdx1-Cre (KPC) mice have been described previously (Hingorani et al., 2005). These mice develop tumours that are similar to human pancreatic tumours in terms of histology and pathology, and are extremely aggressive, often metastatic and highly chemo-resistant, again mimicking human pancreatic cancer. A cohort of KPC mice was established and mice were monitored until they developed pancreatic cancer detectable by palpation. The breeding strategy required to generate these mice, and the experimental design is shown in FIG. 2.

Mice were monitored at least weekly by palpation until pancreatic tumours were detectable. At this time, high resolution ultrasound imaging was used to confirm the presence of pancreatic cancer, and mice were enrolled into cohorts for treatment with the anti-Gremlin1 antibody Ab7326 mIgG1, or Ab7326 mIgG1+Selumetinib in combination (detailed in Tables 3 and 4).

TABLE 3

| TREATMENT | Dose | Schedule | n= | Timepoint |
|---|---|---|---|---|
| Ab7326 mIgG1 | 30 mg/kg s.c. | twice weekly | 4 | 7 days |
| Ab7326 mIgG1 + | 30 mg/kg s.c. | twice weekly | 4 | 7 days |
| Selumetinib | 25 mg/kg p.o. | b.i.d. | | |

TABLE 4

| | | | | Median |
|---|---|---|---|---|
| TREATMENT | Dose | Schedule | n= | survival |
| Ab7326 mIgG1 | 30 mg/kg s.c. | twice weekly | 9 | 19 |
| Selumetinib | 25 mg/kg p.o. | b.i.d. | 8 | 17 |
| Ab7326 mIgG1 + | 30 mg/kg s.c. | twice weekly | 9 | 33 |
| Selumetinib | 25 mg/kg p.o. | b.i.d. | | |

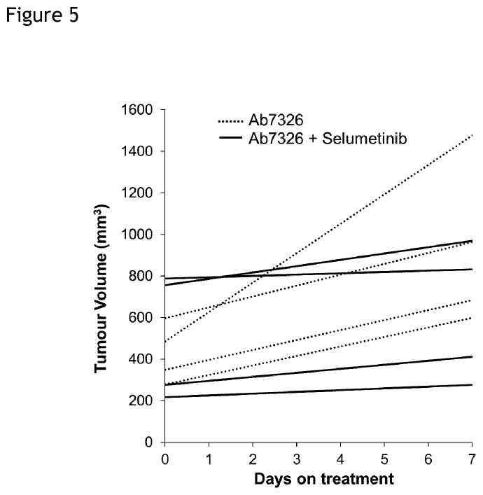

Four mice from each cohort received a follow up ultrasound scan after 1 week and were then sacrificed to generate samples for pharmacodynamic assessment (Table 3). Analysis of gross pathology did not reveal any differences in tumour burden or phenotype between different experimental conditions. Analysis of 3D tumour imaging by ultrasound revealed that tumour growth was slowed in mice treated with Ab7326 mIgG1+Selumetinib in combination (FIG. 5).

### Example 4: Treatment with a Combination of Ab7326 mIgG1 and Selumetinib Results in Slower Tumour Growth and Tumour Shrinkage with Prolonged Response Compared to Treatment with Ab7326 mIgG1 Alone, and a Significant Increase in Median Survival Compared to Treatment with Selumetinib and Ab7326 mIgG1 as Single Agents

For the survival cohort (Table 4), high resolution ultrasound imaging was performed weekly over the treatment period in order to monitor the tumour burden of each individual mouse on treatment. Mice remained on study and were closely monitored until ethical endpoint was reached, at which time they were sacrificed as per institutional guidelines. Clinical features displayed included abdominal distension, loss of body conditioning indicative of cachexia, reduced mobility and occasionally jaundice.

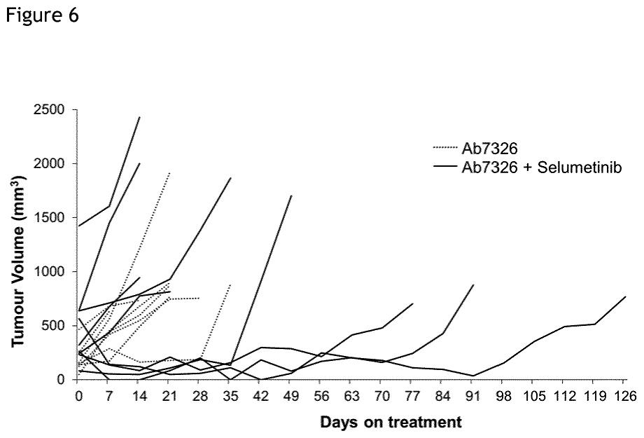

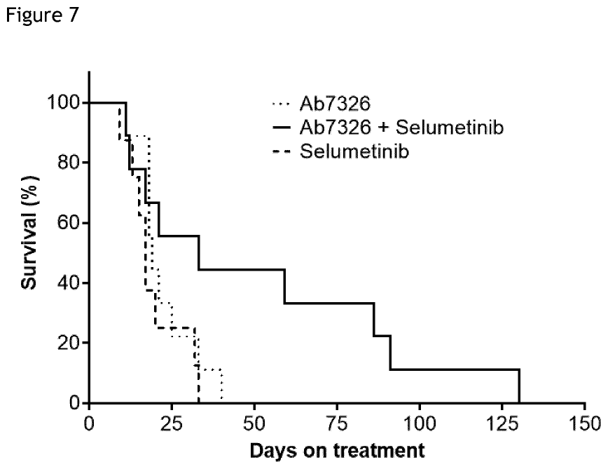

Analysis of 3D tumour imaging by ultrasound revealed that there was slower tumour growth and tumour shrinkage with prolonged response in several mice treated with the combination of Ab7326 mIgG1 and Selumetinib (FIG. 6). In addition, we observed that mice treated with the combination of Ab7326 mIgG1 and Selumetinib showed a significant increase in median survival (33 days) compared with mice treated with Selumetinib alone (17 days, Log Rank, p=0.050), and an increase in median survival compared with mice treated with Ab7326 alone (19 days, see FIG. 7, Table 5, and Table 6 for individual mouse data). Serum samples from both the pharmacodynamic assessment and survival studies were sent to UCB for analysis (Table 7).

TABLE 5

| | |
|---|---|
| Ab7326 + Selumetinib vs. Ab7326 | P = 0.100 |
| Ab7326 + Selumetinib vs. Selumetinib | P = 0.050 |
| Ab7326 + Selumetinib vs. Ab7326 | P = 0.061 |
| (1^{st} & 2^{nd} Ab7326 single | |
| agent cohorts combined) | |

TABLE 6

| | | | | Ab7326 + |
|---|---|---|---|---|
| Mouse ID | Days on Rx | Ab7326 | Selumetinib | Selumetinib |
| NKD207.1d | 11 | 1 | | |
| NKD203.2e | 18 | 1 | | |
| NKD193.7a | 18 | 1 | | |
| NKD203.6a | 18 | 1 | | |
| NKD192.9d | 19 | 1 | | |
| NKD211.1f | 21 | 1 | | |
| NKD211.3e | 25 | 1 | | |
| NKD210.1d | 33 | 1 | | |
| NKD206.2c | 40 | 1 | | |
| 160324 | 9 | | 1 | |
| NKD232.1b | 13 | | 1 | |
| 160323 | 15 | | 1 | |
| NKD214.9e | 17 | | 1 | |
| 130780 | 17 | | 1 | |
| 173159 | 20 | | 1 | |
| 148346 | 32 | | 1 | |
| NKD232.3d | 33 | | 1 | |
| NKD219.1c | 11 | | | 1 |
| NKD217.1f | 12 | | | 1 |
| NKD221.1e | 17 | | | 1 |
| NKD208.1d | 21 | | | 1 |
| NKD214.1e | 33 | | | 1 |
| NKD186.10d | 59 | | | 1 |
| NKD206.1g | 86 | | | 1 |
| NKD190.8e | 91 | | | 1 |
| NKD191.6e | 130 | | | 1 |

TABLE 7

| | | | |
|---|---|---|---|
| NKD191.1g | Ab7326 | 7 days | serum |
| NKD202.3g | Ab7326 | 7 days | serum |
| NKD201.6f | Ab7326 | 7 days | serum |
| NKD219.1d | Ab7326 | 7 days | serum |
| NKD203.2e | Ab7326 | endpoint | serum |
| NKD193.7a | Ab7326 | endpoint | serum |
| NKD192.9d | Ab7326 | endpoint | serum |
| NKD210.1d | Ab7326 | endpoint | serum |
| NKD211.1f | Ab7326 | endpoint | serum |
| NKD206.2c | Ab7326 | endpoint | serum |
| NKD202.1j | Ab7326 + Selumetinib | 7 days | serum |
| NKD203.3e | Ab7326 + Selumetinib | 7 days | serum |
| NKD203.5i | Ab7326 + Selumetinib | 7 days | serum |
| NKD209.2e | Ab7326 + Selumetinib | 7 days | serum |
| NKD186.10d | Ab7326 + Selumetinib | endpoint | serum |
| NKD208.1d | Ab7326 + Selumetinib | endpoint | serum |
| NKD214.1e | Ab7326 + Selumetinib | endpoint | serum |
| NKD221.1e | Ab7326 + Selumetinib | endpoint | serum |
| NKD219.1c | Ab7326 + Selumetinib | endpoint | serum |
| NKD217.1f | Ab7326 + Selumetinib | endpoint | serum |

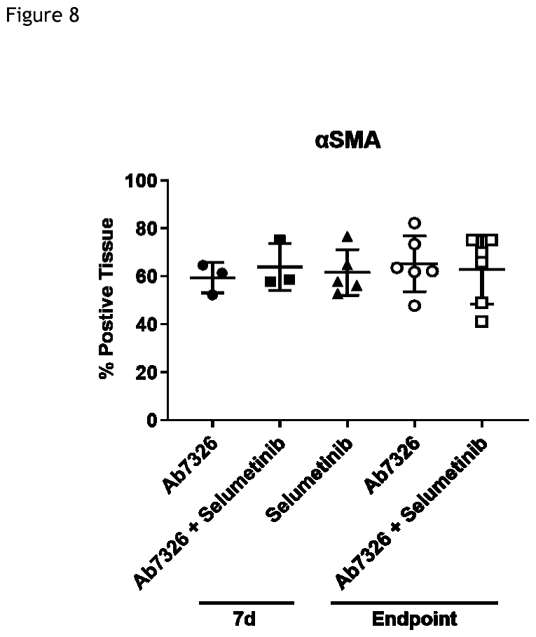

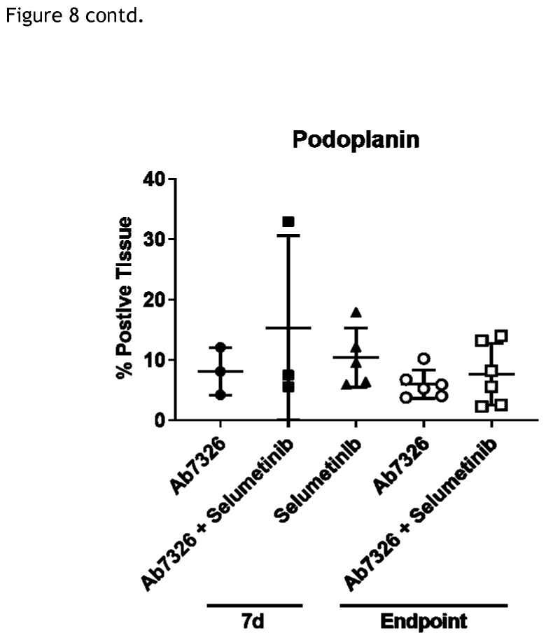

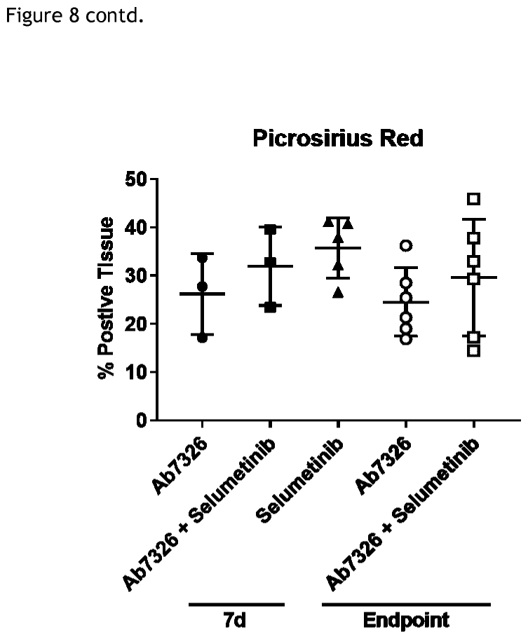

Following sacrifice of experimental mice, examination of gross pathology did not reveal any differences in tumour burden or phenotype between different experimental conditions. Immunohistochemical (IHC) analysis was performed on formalin-fixed paraffin-embedded tumour tissue to examine the tumour microenvironment to assess any changes in the number of alpha-SMA-positive and podoplanin-positive tumour-associated fibroblasts or in the quality or quantity of collagen I and III (as measured by picrosirius red staining). Staining was scored using HALO digital software, however, there was no significant impact on any of these parameters by any of the regimens tested (FIG. 8).

### Results

In summary, the results presented herein demonstrate that MAPK/ERK pathway genes are upregulated upon exposure with Ab7326 in a KPC mouse model. Moreover, Ab7326 mIgG1 can be given safely to KPC mice in combination with Selumetinib. A significant improvement in survival was observed when mice were treated with a combination of Ab7326 mIgG1 and Selumetinib compared to treatment with Selumetinib alone (see FIG. 7, Table 5, and data for individual mice in Table 6). Moreover, tumour shrinkage was observed in several mice treated with a combination of Ab7326 mIgG1 and Selumetinib, and responses were prolonged in these mice. This suggests that the combination of Ab7326 mIgG1 and Selumetinib represents a highly promising treatment for pancreatic cancer.

### Example 5: Treatment of KPC Mice with a Combination of Ab7326 mIgG1 with Either Selumetinib or UCB-554 MEK Inhibitors Results in Slower Tumour Growth and Prolonged Survival Compared to Treatment with Single Agents

To determine if the prolonged survival seen with combination of Ab7326 and Selumetinib in Example 3 is applicable to other MEK inhibitors, and to further increase the robustness of the study by increasing the numbers of KPC mice in each cohort, a study was run comparing Ab7326 in combination with either the MEK inhibitor Selumetinib or the MEK inhibitor UCB-554. KPC mice with confirmed pancreatic tumours were established as described in Materials and Methods, enrolled into cohorts for treatment as shown in Table 8 and monitored for tumour growth by ultrasound and ethical survival endpoints.

TABLE 8

| | | | | Median |
|---|---|---|---|---|
| TREATMENT | Dose | Schedule | n= | survival |
| Vehicle | 125 μl p.o. | every 2 days | 10 | 12 days |
| Ab7326 mIgG1 | 30 mg/kg s.c. | twice weekly | 10 | 9.5 days |
| Selumetinib | 25 mg/kg p.o. | b.i.d. | 9 | 22 days |
| Ab7326 mIgG1 + | 30 mg/kg s.c. | twice weekly | 10 | 29.5 days |
| Selumetinib | 25 mg/kg p.o. | b.i.d. | | |
| UCB-554 | 10 mg/kg p.o. | every 2 days | 10 | 21 days |
| Ab7326 mIgG1 + | 30 mg/kg s.c. | twice weekly | 11 | 32 days |
| UCB-554 | 10 mg/kg p.o. | every 2 days | | |

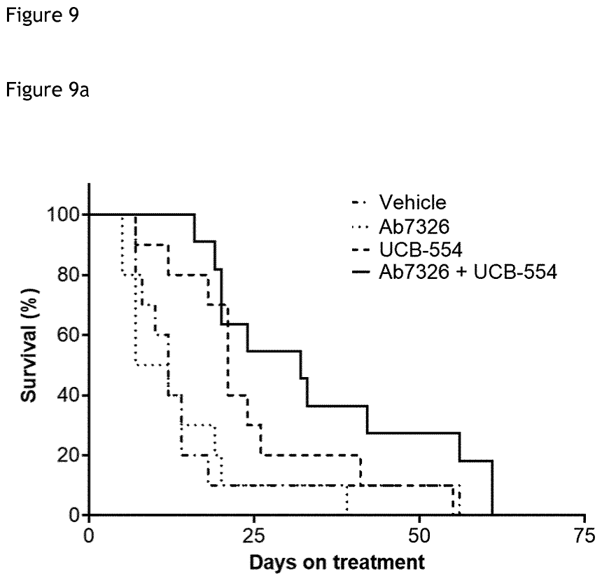

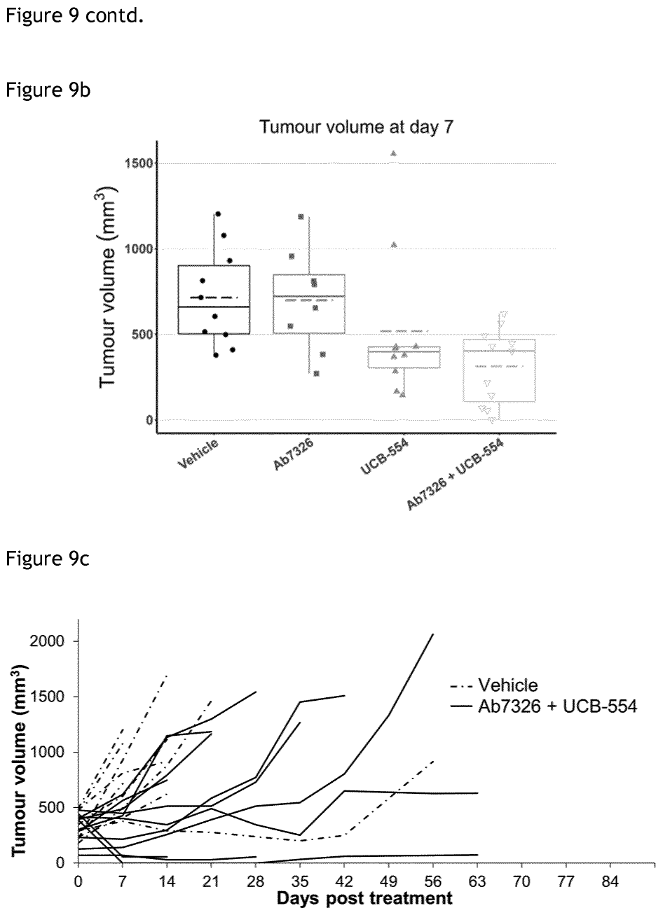

Treatment with the MEK inhibitor UCB-554 led to a moderate increase in survival of the KPC mice over vehicle only control or Ab7326 treatment (FIG. 9a and Table 8 median survival). However, combination treatment with Ab7326 and UCB-554 led to further improved survival of the KPC mice compared to UCB-554 alone (FIG. 9a and Table 8 median survival). A Cox's proportional hazards model was fitted to the survival data and the hazard ratios (HR) were estimated for the comparison of different treatment groups (Table 9). The hazard ratios for Ab7326+UCB-554 versus vehicle control (HR=3.57, p =0.005) or Ab7326 alone (HR=4.38, p=0.001) demonstrated a statistically significant improvement of survival with the combination. The hazard ratio for Ab7326+UCB-554 versus UCB-554 alone was also greater than one (HR=1.75) suggesting an improved survival rate with the combination over MEK inhibition alone but did not reach statistical significance (p=0.211).

TABLE 9
Summary of survival statistics for KPC mice
treated with Ab7326 and UCB-554 combinations

| Comparison | Hazard ratio | P value |
|---|---|---|
| Vehicle/Ab7326 | 0.82 | 0.656 |
| Vehicle/UCB-554 | 2.04 | 0.120 |
| Vehicle/Ab7326 + UCB-554 | 3.57 | 0.005 |
| Ab7326/UCB-554 | 2.50 | 0.043 |
| Ab7326/Ab7326 + UCB-554 | 4.38 | 0.001 |
| UCB-554/Ab7326 + UCB-554 | 1.75 | 0.211 |

The volume of the pancreatic tumours in control or treated KPC mice was measured over time using 3D tumour imaging by ultrasound. Comparison of the tumour volume at day 7 after start of treatment showed that, in agreement with the survival data, the tumour volume is reduced in the UCB-554 treated group compared to vehicle or Ab7326 treatment alone (FIG. 9b). Furthermore, the combination of Ab7326+UCB-554 had a statistically significant reduction in tumour volume at day 7 compared to UCB-554 alone (p=0.040).

Measurement of the tumour volumes over time of the mice in this cohort, until ethical endpoint, indicated that the combined treatment of Ab7326 and UCB-554 led to shrinkage or reduced tumour growth in a subset of mice compared to vehicle controls (FIG. 9c).

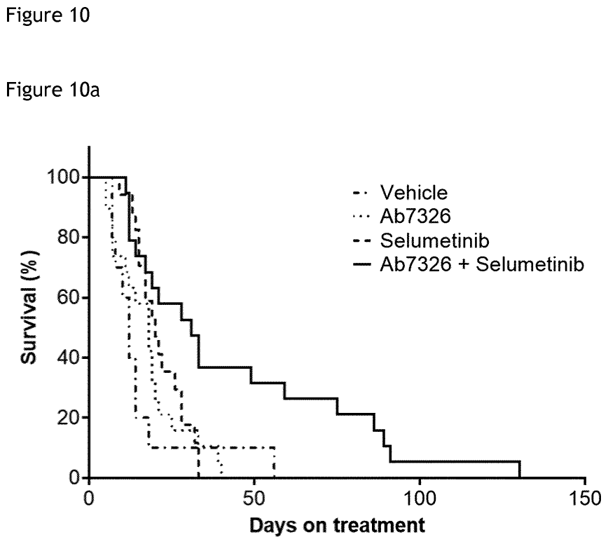

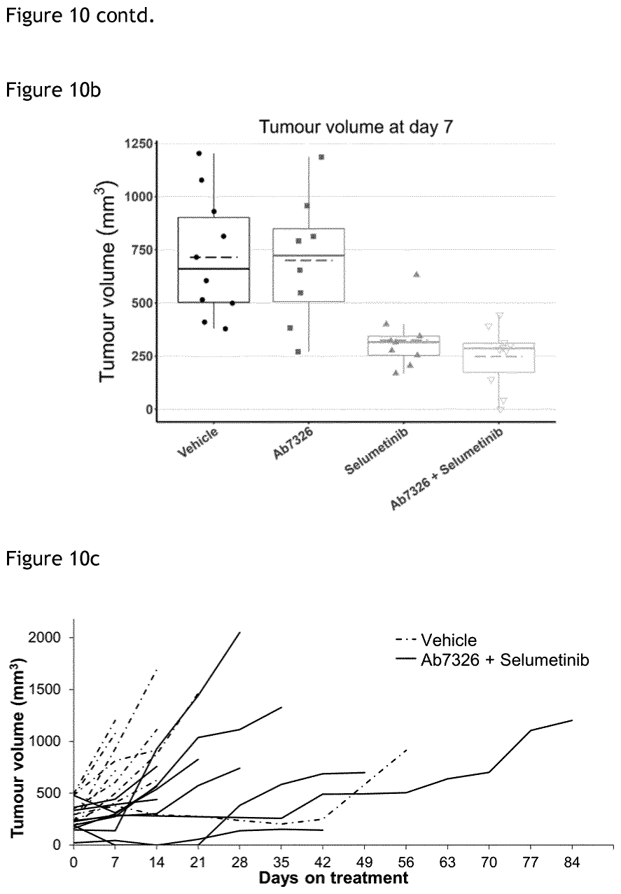

The KPC mouse survival data collected in the Selumetinib MEK inhibitor study shown in Example 3 (Table 4) was combined with the data from the Example 5 cohort (Table 8) to increase the robustness of the analysis by including greater numbers of animals. As shown in FIG. 10a and Table 10, combination of Ab7326 and Selumetinib significantly increased survival of KPC mice over vehicle alone (HR=5.16, p<0.001) or Ab7326 treatment (HR=3.82, p<0.001). Moreover, combination of Ab7326 and Selumetinib significantly increased survival over treatment with Selumetinib alone, with a hazard ratio of 2.92 (p=0.006) indicating there is a significant beneficial effect of combining inhibition of gremlin-1 with MEK pathway inhibition in this model of pancreatic cancer.

TABLE 10
Summary of survival statistics for KPC mice treated with Ab7326 and
Selumetinib combinations

| Comparison | Hazard ratio | P value |
|---|---|---|
| Vehicle/Ab7326 | 1.35 | 0.450 |
| Vehicle/Selumetinib | 1.77 | 0.166 |
| Vehicle/Ab7326 + Selumetinib | 5.16 | 0.000 |
| Ab7326/Selumetinib | 1.31 | 0.427 |
| Ab7326/Ab7326 + Selumetinib | 3.82 | 0.000 |
| Selumetinib/Ab7326 + | 2.92 | 0.006 |
| Selumetinib | | |

Comparison of the tumour volume at day 7 after start of treatment showed that, in agreement with the survival data, the mean tumour volume is reduced in the Selumetinib and the Selumetinib+Ab7326 treated groups compared to vehicle or Ab7326 treatment alone (FIG. 10b).

Measurement of the tumour volumes over time of the mice in this cohort, until ethical endpoint, indicated that the combined treatment of Ab7326 and Selumetinib led to shrinkage or reduced tumour growth in a subset of mice compared to vehicle controls (FIG. 10c).

In conclusion, these data demonstrate that the anti-gremlin-1 antibody Ab7326 dosed in combination with a MEK inhibitor such as Selumetinib or UCB-554 leads to reduced tumour growth and increased survival over treatment with individual agents in the KPC murine model of pancreatic cancer. This suggests that targeting gremlin-1 and MEK pharmacological pathways simultaneously may have additive or synergistic effects in the treatment of pancreatic cancer.

### Aspects

1. An anti-GREM1 antagonist for use in a method for the treatment or prevention of a cancer, wherein the method further comprises administering an inhibitor of Ras-Raf-MEK-ERK signalling.
2. The anti-GREM1 antagonist for use according to aspect 1, wherein the cancer is a solid cancer.
3. The anti-GREM1 antagonist for use according to aspect 1 or aspect 2, wherein the cancer has stromal GREM1 overexpression.
4. The anti-GREM1 antagonist for use according to any one of the preceding aspects, wherein the cancer is a cancer associated with Ras-Raf-MEK-ERK pathway dysregulation.
5. The anti-GREM1 antagonist for use according to any one of the preceding aspects, wherein the cancer displays Ras-Raf-MEK-ERK pathway dysregulation following exposure to an anti-GREM1 antagonist.
6. The anti-GREM1 antagonist for use according to any one of the preceding aspects, wherein the cancer contains a mutation in a Ras gene optionally KRAS, NRAS and/or HRAS.
7. The anti-GREM1 antagonist for use according to any one of the preceding aspects, wherein the cancer contains a mutation in a Raf gene; optionally ARAF, BRAF and/or CRAF.
8. The anti-GREM1 antagonist for use according to any one of the preceding aspects, wherein the cancer is a cancer that is poorly responsive, non-responsive or refractory to treatment with an inhibitor of Ras-Raf-MEK-ERK signalling.
9. The anti-GREM1 antagonist for use according to any one of the preceding aspects, wherein the cancer is selected from colorectal cancer, multiple myeloma, pancreatic cancer, bladder cancer, breast cancer, lung cancer, stomach cancer, duodenal cancer, oesophageal cancer, head and neck cancer, prostate cancer, glioma, endometrial cancer, liver cancer, spleen cancer, bone-resident cancer, melanoma and osteosarcoma.
10. The anti-GREM1 antagonist for use according to aspect 9, wherein the cancer is pancreatic cancer.
11. The anti-GREM1 antagonist for use according to aspect 10, wherein the pancreatic cancer is an exocrine pancreatic cancer.
12. The anti-GREM1 antagonist for use according to aspect 10 or aspect 11, wherein the pancreatic cancer is pancreatic ductal adenocarcinoma (PDAC).
13. The anti-GREM1 antagonist for use according to aspect 9, wherein the cancer is multiple myeloma or colorectal cancer; optionally KRAS/BRAF mutated colorectal cancer.
14. The anti-GREM1 antagonist for use according to aspect 9, wherein the cancer is lung cancer; optionally wherein the cancer is a non-small cell lung cancer (NSCLC).
15. The anti-GREM1 antagonist for use according to aspect 9, wherein the cancer is a melanoma; optionally a BRAF-mutated melanoma.
16. The anti-GREM1 antagonist for use according to any one of the preceding aspects, wherein the cancer has epithelial GREM1 overexpression.
17. The anti-GREM1 antagonist for use according to aspect 16, wherein the cancer is a GREM1-initiated cancer.
18. The anti-GREM1 antagonist for use according to any one of the preceding aspects, wherein the cancer is a disseminated cancer.
19. The anti-GREM1 antagonist for use according to any one of the preceding aspects, wherein the cancer is an established cancer.
20. The anti-GREM1 antagonist for use according to any one of the preceding aspects, wherein the antagonist is a peptide, a protein, an antibody, a polynucleotide, an oligonucleotide, an antisense RNA, a small interfering RNA (siRNA), a small molecule inhibitor or a small hairpin RNA (shRNA).
21. The anti-GREM1 antagonist for use according to aspect 20, wherein the antagonist is an antibody which binds to an epitope on Gremlin-1 comprising at least one residue selected from Ile131, Lys147, Lys148, Phe149, Thr150, Thr151, Arg169, Lys174 and Gln175, wherein the residue numbering is according to SEQ ID NO: 1.
22. The anti-GREM1 antagonist for use according to aspect 21, wherein the antibody binds an epitope comprising all of Ile131, Lys147, Lys148, Phe149, Thr150, Thr151, Arg169, Lys174 and Gln175.
23. The anti-GREM1 antagonist for use according to aspect 21 or 22, wherein Lys147, Lys148, Phe149, Thr150, Thr151, Arg169, Lys174 and Gln175 are located on the same Gremlin-1 monomer and Ile131 is located on the second Gremlin-1 monomer.
24. The anti-GREM1 antagonist for use according to aspect 20, wherein the antagonist is an anti-Gremlin-1 antibody which comprises heavy chain complementarity determining region (HCDR) sequences contained within a heavy chain variable region (HCVR) of SEQ ID NO: 10 or 12 and/or light chain complementarity determining region (LCDR) sequences contained within a light chain variable region (LCVR) of SEQ ID NO: 11 or 13.
25. The anti-GREM1 antagonist for use according to aspect 20, wherein the antagonist is an anti-Gremlin-1 antibody which comprises at least one HCDR sequence selected from SEQ ID NOs: 3, 4, 5 and 6 and/or at least one LCDR sequence selected from SEQ ID NOs: 7, 8 and 9.
26. The anti-GREM1 antagonist for use according to aspect 25, wherein the anti-Gremlin-1 antibody comprises a HCDR3 sequence of SEQ ID NO: 6.
27. The anti-GREM1 antagonist for use according to aspect 25 or 26, wherein the anti-Gremlin-1 antibody comprises an HCDR1/HCDR2/HCDR3 sequence combination selected from SEQ ID NOs: 4/5/6 or from SEQ ID NOs:3/5/6 and/or an LCDR1/LCDR2/LCDR3 sequence combination selected from SEQ ID NOs: 7/8/9.
28. The anti-GREM1 antagonist for use according to any one of aspects 25 to 27, wherein the anti-Gremlin-1 antibody comprises a HCDR1/HCDR2/HCDR3/LCDR1/LCDR2/LCDR3 sequence combination of SEQ ID NOs: 4/5/6/7/8/9 or SEQ ID NOs: 3/5/6/7/8/9.
29. The anti-GREM1 antagonist for use according to any one of aspects 25 to 28, wherein the anti-Gremlin-1 antibody comprises a heavy chain variable region (HCVR) sequence of SEQ ID NO: 10 or 12 and/or a light chain variable region (LCVR) sequence of SEQ ID NO: 11 or 13, or sequences which are at least 95% identical thereto.
30. The anti-GREM1 antagonist for use according to aspect 29, wherein the anti-Gremlin-1 antibody comprises a HCVR and LCVR sequence pair of SEQ ID NOs: 10/11 or 12/13 or sequences which are at least 95% identical thereto.
31. The anti-GREM1 antagonist for use according to aspect 30, wherein the anti-Gremlin-1 antibody comprises HCDR1/HCDR2/HCDR3/LCDR1/LCDR2/LCDR3 sequences consisting of SEQ ID NOs: 4/5/6/7/8/9 or SEQ ID NOs: 3/5/6/7/8/9 and the remainder of the HCVR and LCVR comprise at least 95% identity to SEQ ID NOs: 10, 11, 12 and/or 13 respectively.
32. The anti-GREM1 antagonist for use according to aspects 29-31, wherein the anti-Gremlin-1 antibody comprises a heavy chain of SEQ ID NO: 14, 16, 18, 22, 28, 30, 32 or 34 and/or a light chain of SEQ ID NO: 15, 17, 19, 23, 29, 31, 33 or 35, or sequences which are at least 95% identical thereto.
33. The anti-GREM1 antagonist for use according to aspect 32, wherein the anti-Gremlin-1 antibody comprises a heavy and light chain pair of SEQ ID NOs: 14/15, 16/17, 18/19, 22/23, 28/29 or 30/31, 32/33, 34/35, or sequences which are at least 95% identical thereto.
34. The anti-GREM1 antagonist for use according to aspect 33, wherein the HCDR1/HCDR2/HCDR3/LCDR1/LCDR2/LCDR3 sequences of the antibody consist of SEQ ID NOs: 4/5/6/7/8/9 or SEQ ID NOs: 3/5/6/7/8/9 and the remainder of the heavy and light chains comprise at least 95% identity to SEQ ID NOs: 14, 15, 16 and/or 17 respectively.
35. The anti-GREM1 antagonist for use according to aspect 20, wherein the antagonist is an antibody which competes for binding to Gremlin-1 with an antibody as defined in any one of aspects 22-32.
36. The anti-GREM1 antagonist for use according to aspect 20, wherein the antagonist is an antibody which binds the same epitope on Gremlin-1 as an antibody defined in any one of aspects 22-32.
37. The anti-GREM1 antagonist for use according to any one of aspects 20-36, wherein the antagonist antibody is a chimeric, human or humanised antibody.
38. The anti-GREM1 antagonist for use according to any one of aspects 20-37, wherein the antagonist antibody is a Fab, modified Fab, Fab′, modified Fab′, F(ab′)2, Fv, single domain antibody or an scFv.
39. The anti-GREM1 antagonist for use according to aspect 20, wherein the antagonist is a polynucleotide encoding an antibody as defined in any one of aspects 21-38, or an expression vector carrying said polynucleotide.
40. The anti-GREM1 antagonist for use according to any one of aspects 20-38, wherein the antagonist antibody is comprised in pharmaceutical composition further comprising a pharmaceutically acceptable adjuvant and/or carrier.
41. The anti-GREM1 antagonist for use according to any one of the preceding aspects wherein the inhibitor of Ras-Raf-MEK-ERK signalling downregulates Mapk1, Nras, Kras and/or Myc signalling.
42. The anti-GREM1 antagonist for use according to any one of the preceding aspects wherein the inhibitor of Ras-Raf-MEK-ERK signalling is a MEK inhibitor; optionally an allosteric MEK inhibitor.
43. The anti-GREM1 antagonist for use according to aspect 42 wherein the MEK inhibitor is a MEK1 and/or MEK2 inhibitor.
44. The anti-GREM1 antagonist for use according to aspect 42 or aspect 43, wherein the MEK inhibitor is selumetinib (AZD6244), trametinib (GSK-1120212), cobimetinib (GDC-0973), binimetinib (MEK162), CI-1040 (PD184352), Mirdametinib (PD0325901), TAK733, refametinib (RDEA119/Bay 86-9766), RO-5126766, RO5068760, Pimasertib (AS703026), AZD8330, GDC-0623, RO-4987655, WX-554 (UCB-554), HL-085, ARRY-300, ClnQ-03, G-573, PD184161, PD318088, PD98059, U0126 or SL327.
45. The anti-GREM1 antagonist for use according to aspect 42, wherein the MEK inhibitor is selected from the group consisting of:
   (a) N-(2-fluoro-4-iodophenyl)-3-(morpholin-4-ylcarbonyl)thieno[2,3-b]pyridin-2-amine;
   (b) N-(2-fluoro-4-iodophenyl)-3-[(4-methylpiperazin-1-yl)carbonyl]thieno[2,3-b]pyridin-2-amine;
   (c) [2-[((2-fluoro-4-iodophenyl)amino]thieno[2,3-b]pyridin-3-yl](pyrrolidin-1-yl)methanone;
   (d) (1-{2-[(2-fluoro-4-iodophenyl)amino]thieno[2,3-b]pyridine-3-carbonyl}piperidin-4-yl)-carbamic acid tert-butyl ester;
   (e) 2-[((2-fluoro-4-iodophenyl)amino]thieno[2,3-b]pyridine-3-carboxylic acid (4-aminopiperidin-1-yl)amide dihydro-chloride;
   (f) (1-{2-[(2-fluoro-4-iodophenyl)amino]thieno[2,3-b]pyridine-3-carbonyl}azetidin-3-ylmethyl)carbamic acid tert-butyl ester;
   (g) [3-((aminomethyl)azetidin-1-yl]-{2-[(2-fluoro-4-iodophenyl)amino]thieno[2,3-b]pyridin-3-yl}-methanone;
   (h) (1-{2-[(2-fluoro-4-iodophenyl)amino]thieno[2,3-b]pyridine-3-carbonyl}-(3R)-pyrrolidin-3-yl)carbamic acid tert-butyl ester;
   (i) (1-{2-[(2-fluoro-4-iodophenyl)amino]thieno[2,3-b]pyridine-3-carbonyl}-(3S)-pyrrolidin-3-yl)carbamic acid tert-butyl ester;
   (j) (1-{2-[(2-fluoro-4-iodophenyl)amino]thieno[2,3-b]pyridine-3-carbonyl}azetidin-3-yl)-carbamic acid tert-butyl ester;
   (k) [(3R)-3-aminopyrrolidin-1-yl]-{2-[(2-fluoro-4-iodophenyl)amino]thieno[2,3-b]pyridin-3-yl}-methanone;
   (l) [(3S)-3-aminopyrrolidin-1-yl]-{2-[(2-fluoro4-iodophenyl)amino]thieno[2,3-b]pyridin-3-yl}-methanone;
   (m) (3-aminoazetidin-1-yl)-{2-[(2-fluoro-4-iodophenyl)amino]thieno[2,3-b]pyridin-3-yl]-methanone;
   (n) {2-[((2-fluoro-4-iodophenyl)amino]thieno[2,3-b]pyridin-3-yl}-(3-hydroxyazetidin-1-yl)-methanone;
   (o) {2-[((2-fluoro-4-iodophenyl)amino]thieno[2,3-b]pyridin-3-yl}-[(3R)-3-hydroxypyrrolidin-1-yl]-methanone;
   (p) {2-[((2-fluoro-4-iodophenyl)amino]thieno[2,3-b]pyridin-3-yl}-[(3S)-3-hydroxypyrrolidin-1-yl]-methanone;
   (q) {2-[((2-fluoro-4-iodophenyl)amino]thieno[2,3-b]pyridin-3-yl}-[2-(hydroxymethyl)-piperidin-1-yl]-methanone;
   (r) {2-[((2-fluoro-4-iodophenyl)amino]thieno[2,3-b]pyridin-3-yl}-[(3S)-3-(hydroxymethyl)-morpholin-4-yl]-methanone;
   (s) 4-{2-[(2-fluoro-4-iodophenyl)amino]thieno[2,3-b]pyridine-3-carbonyl}-(3R)-3-(hydroxymethyl)piperazine-1-carboxylic acid tert-butyl ester;
   (t) 2-[4({2-[(2-fluoro-4-iodophenyl)amino]thieno[2,3-b]pyridin-3-yl}carbonyl)piperazin-1-yl]ethanol;
   (u) 3-((1,4-diazepan-1-ylcarbonyl)-N-(2-fluoro-4-iodophenyl)thieno[2,3-b]pyridin-2-amine;
   (v) N-(2-fluoro-4-iodophenyl)-3-(piperazin-1-ylcarbonyl)thieno[2,3-b]pyridin-2-amine;
   (w) ethyl [4-({2-[(2-fluoro-4-iodophenyl)amino]thieno[2,3-b]pyridin-3-yl}carbonyl)-piperazin-1-yl]acetate;
   (x) [4-({2-[(2-fluoro-4-iodophenyl)amino]thieno[2,3-b]pyridin-3-yl}carbonyl)piperazin-1-yl]acetic acid;
   (y) {2-[((2-fluoro-4-iodophenyl)amino]thieno[2,3-b]pyridin-3-yl}-[(2R)-2-(methoxymethyl)-pyrrolidin-1-yl]-methone; and
   (z) {2-[((2-fluoro-4-iodophenyl)amino]thieno[2,3-b]pyridin-3-yl}-[(2R)-2-(hydroxymethyl)-piperazin-1-yl]-methanone.
46. The anti-GREM1 antagonist for use according to aspect 42, wherein the MEK inhibitor is represented by formula (II), and pharmaceutically acceptable salts, solvates and N-oxides thereof:

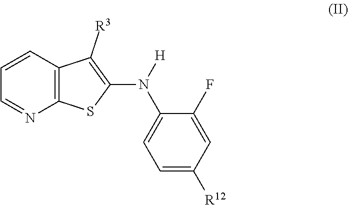

wherein
R¹² represents halogen;
R³ represents CONR^{b}R^{c}; and
R^{b} and R^{c}, when taken together with the nitrogen atom to which they are both attached, represent azetidin-1-yl, pyrrolidin-1-yl, piperidin-1-yl, morpholin-4-yl, thiomorpholin-4-yl, piperazin-1-yl, homopiperidin-l-yl, homo-morpholin-4-yl or homopiperazin-1-yl, any of which groups may be optionally substituted by one or more substituents selected from C₁₋₆ alkyl, C₁₋₆ alkoxy, hydroxy, hydroxy(C₁_6)akyl, amino-(C₁₋₆)alkyl, (amino)(hydroxy)(C₁₋₆)alkyl, halogen, oxo, C₂₋₆ akylcarbonyl, carboxy, C₂₋₆ alkoxycarbonyl, di(C₁₋₆)alhylhydrazinylcarbonyl, amino, C₁₋₆ alkylamino, di(C₁₋₆)alkyl-amino, C₂₋₆ alkylcarbonylamino, aminocarbonylamino, aminocarbonyl, C₁₋₆ alkylaminocarbonyl, di(C₁₋₆)alkylaminocarbonyl, aminosulfonyl, C₁₋₆ alkylsulfonyl, C₁₋₆ alkylaminocarbonyl (C₁₋₆)alkyl, C₁₋₆ alkoxy(C₁₋₆)alkyl, carboxy(C₁₋₆)alkyl, C₂₋₆ alkoxycarbonyl(C₁₋₆)alkyl, C₂₋₆ alkoxycarbonylamino and C₂₋₆ alkoxycarbonylamino-(C₁₋₆)alkyl.
47. The anti-GREM1 antagonist for use according to claim 46, wherein R¹² represents bromo or iodo.
48. The anti-GREM1 antagonist for use according to aspect 44, wherein the MEK inhibitor is selumetinib (AZD6244).
49. The anti-GREM1 antagonist for use according to aspect 44, wherein the MEK inhibitor is trametinib (GSK-1120212).
50. The anti-GREM1 antagonist for use according to aspect 44, wherein the MEK inhibitor is WX-554 (UCB-554).
51. The anti-GREM1 antagonist for use according to aspect 44, wherein the MEK inhibitor is selumetinib (AZD6244) and the cancer is pancreatic cancer; optionally PDAC.
52. The anti-GREM1 antagonist for use according to aspect 44, wherein the MEK inhibitor is trametinib (GSK-1120212) and the cancer is pancreatic cancer; optionally PDAC.
53. The anti-GREM1 antagonist for use according to aspect 44, wherein the MEK inhibitor is WX-554 (UCB-554) and the cancer is pancreatic cancer; optionally PDAC.
54. The anti-GREM1 antagonist for use according to any one of aspects 1 to 41, wherein the inhibitor of Ras-Raf-MEK-ERK signalling is an ERK inhibitor.
55. The anti-GREM1 antagonist for use according to aspect 54, wherein the ERK inhibitor is ulixertinib, temuterkib, JSI-1187, ravoxertinib, ASN007 or MK-8353.
56. The anti-GREM1 antagonist for use according to aspect 54 or 55, wherein the ERK inhibitor is ulixertinib.
57. The anti-GREM1 antagonist for use according to aspect 56, wherein the ERK inhibitor is ulixertinib and the cancer is pancreatic cancer; optionally PDAC.
58. The anti-GREM1 antagonist for use according to any one of the preceding aspects, wherein the method further comprises administering an additional anti-cancer agent.
59. An inhibitor of Ras-Raf-MEK-ERK signalling for use in a method for the treatment or prevention of cancer wherein the method further comprises administering an anti-GREM1 antagonist.
60. The inhibitor of Ras-Raf-MEK-ERK for use according to aspect 59, wherein said cancer, said antagonist and/or said method are as defined in any one of aspects 1-58.
61. The inhibitor of Ras-Raf-MEK-ERK signalling for use according to aspect 59 or 60, wherein the inhibitor of Ras-Raf-MEK-ERK signalling is a MEK inhibitor.
62. The inhibitor of Ras-Raf-MEK-ERK signalling for use according to aspect 61, wherein said MEK inhibitor is selumetinib (AZD6244).
63. The inhibitor of Ras-Raf-MEK-ERK signalling for use according to aspect 61, wherein said MEK inhibitor is trametinib (GSK-1120212).
64. The inhibitor of Ras-Raf-MEK-ERK signalling for use according to aspect 61, wherein said MEK inhibitor is WX-554 (UCB-554).
65. The inhibitor of Ras-Raf-MEK-ERK signalling for use according to aspect 59 or 60, wherein the inhibitor of Ras-Raf-MEK-ERK signalling is an ERK inhibitor.
66. The inhibitor of Ras-Raf-MEK-ERK signalling for use according to aspect 65, wherein said ERK inhibitor is ulixertinib.
67. A method of treating a cancer comprising administering a therapeutically effective amount of an anti-GREM1 antagonist in combination with a therapeutically effective amount of an inhibitor of Ras-Raf-MEK-ERK signalling to a subject in need thereof.
68. The method according to aspect 67, wherein said cancer, said antagonist and/or said method are as defined in any one of aspects 1-66.
69. A composition or kit comprising an anti-GREM1 antagonist and an inhibitor of Ras-Raf-MEK-ERK signalling.
70. The composition or kit according to aspect 69, wherein the anti-GREM1 antagonist is as defined in any one of aspects 20 to 39.
71. The composition or kit according to aspect 69 or 70, wherein the inhibitor of Ras-Raf-MEK-ERK signalling is as defined in any one of aspects 41 to 57.
72. A method for determining whether or not a patient having or suspected of having or being at risk of developing cancer is likely to respond to a combination treatment with a GREM1 antagonist and an inhibitor of Ras-Raf-MEK-ERK signalling, which method comprises measuring stromal expression of GREM1, expithelial expression of GREM1 and/or Ras-Raf-MEK-ERK pathway signalling in the patient, and thereby predicting whether or not the patient is likely to respond to treatment with the combination.
73. A method for determining whether or not a patient having or suspected of having or being at risk of developing cancer is likely to respond to a combination treatment with a GREM1 antagonist and an inhibitor of Ras-Raf-MEK-ERK signalling, which method comprises measuring stromal expression of GREM1 and/or epithelial expression of GREM1 in the patient and determining whether the patient has a mutation in a RAS gene or a RAF gene, and thereby predicting whether or not the patient is likely to respond to treatment with the combination.
74. A method for determining whether or not a patient having or suspected of having or being at risk of developing cancer is likely to respond to a combination treatment with a GREM1 antagonist and an inhibitor of Ras-Raf-MEK-ERK signalling, which method comprises measuring Ras-Raf-MEK-ERK pathway signalling in the patient in response to treatment with an anti-GREM1 antagonist, and thereby predicting whether or not the patient is likely to respond to treatment with the combination.

SEQUENCE LISTING
(Human Gremlin-1; Uniprot ID: 060565)
SEQ ID NO: 1
MSRTAYTVGALLLLLGTLLPAAEGKKKGSQGAIPPPDKAQHNDSEQTQSPQQPGS
RNRGRGQGRGTAMPGEEVLESSQEALHVTERKYLKRDWCKTQPLKQTIHEEGCNS
RTIINRFCYGQCNSFYIPRHIRKEEGSFQSCSFCKPKKFTTMMVTLNCPELQPPT
KKKRVTRVKQCRCISIDLD
(Human truncated Gremlin-1 used in crystallography with N-terminal
tag)
SEQ ID NO: 2
MGSSHHHHHHSSGENLYFQGSAMPGEEVLESSQEALHVTERKYLKRDWCKTQPLK
QTIHEEGCNSRTIINRFCYGQCNSFYIPRHIRKEEGSFQSCSFCKPKKFTTMMVT
LNCPELQPPTKKKRVTRVKQCRCISIDLD
(Ab7326 HCDR1 combined Kabat & Chothia)
SEQ ID NO: 3
GYTFTDYYMH
(Ab7326 HCDR1 Kabat)
SEQ ID NO: 4
DYYMH
(Ab7326 HCDR2 Kabat)
SEQ ID NO: 5
LVDPEDGETIYAEKFQG
(Ab7326 HCDR3 Kabat)
SEQ ID NO: 6
DARGSGSYYPNHFDY
(Ab7326 LCDR1 Kabat)
SEQ ID NO: 7
KSSQSVLYSSNNKNYLA
(Ab7326 LCDR2 Kabat)
SEQ ID NO: 8
WASTRES
(Ab7326 LCDR3 Kabat)
SEQ ID NO: 9
QQYYDTPT
(Ab7326 Heavy chain variable region variant 1)
SEQ ID NO: 10
QVQLVESGAEVKKPGATVKISCKVSGYTFTDYYMHWVQQAPGKGLEWMGLVDPED
GETIYAEKFQGRVTITADTSTDTAYMELSSLRSEDTAVYYCATDARGSGSYYPNH
FDYWGQGTLVTVSS
(Ab7326 Light chain variable region variant 1)
SEQ ID NO: 11
DIVMTQSPDSLAVSLGERATINCKSSQSVLYSSNNKNYLAWYQQKPGQPPKLLIY
WASTRESGVPDRFSGSGSGTDFTLTINSLQAEDVAVYFCQQYYDTPTFGQGTRLE
IK
(Ab7326 Heavy chain variable region variant 2)
SEQ ID NO: 12
QVQLVQSGAEVKKPGATVKISCKVSGYTFTDYYMHWVQQAPGKGLEWMGLVDPED
GETIYAEKFQGRVTITADTSTDTAYMELSSLRSEDTAVYYCATDARGSGSYYPNH
FDYWGQGTLVTVSS
(Ab7326 Light chain variable region variant 2)
SEQ ID NO: 13
DIVMTQTPDSLAVSLGERATINCKSSQSVLYSSNNKNYLAWYQQKPGQPPKLLIY
WASTRESGVPDRESGSGSGTDFTLTINSLQAEDVAVYFCQQYYDTPTFGQGTRLE
IK
(Mouse full length IgG1 heavy chain variant 1)
SEQ ID NO: 14
QVQLVESGAEVKKPGATVKISCKVSGYTFTDYYMHWVQQAPGKGLEWMGLVDPED
GETIYAEKFQGRVTITADTSTDTAYMELSSLRSEDTAVYYCATDARGSGSYYPNH
FDYWGQGTLVTVSSAKTTPPSVYPLAPGSAAQTNSMVTLGCLVKGYFPEPVTVTW
NSGSLSSGVHTFPAVLQSDLYTLSSSVTVPSSTWPSETVTCNVAHPASSTKVDKK
IVPRDCGCKPCICTVPEVSSVFIFPPKPKDVLTITLTPKVTCVVVDISKDDPEVQ
FSWFVDDVEVHTAQTQPREEQFNSTFRSVSELPIMHQDWLNGKEFKCRVNSAAFP
APIEKTISKTKGRPKAPQVYTIPPPKEQMAKDKVSLTCMITDFFPEDITVEWQWN
GQPAENYKNTQPIMDTDGSYFVYSKLNVQKSNWEAGNTFTCSVLHEGLHNHHTEK
SLSHSPGK
(Mouse full length IgG1 light chain variant 1)
SEQ ID NO: 15
DIVMTQSPDSLAVSLGERATINCKSSQSVLYSSNNKNYLAWYQQKPGQPPKLLIY
WASTRESGVPDRFSGSGSGTDFTLTINSLQAEDVAVYFCQQYYDTPTFGQGTRLE
IKRTDAAPTVSIFPPSSEQLTSGGASVVCFLNNFYPKDINVKWKIDGSERQNGVL
NSWTDQDSKDSTYSMSSTLTLTKDEYERHNSYTCEATHKTSTSPIVKSFNRNEC
(Human full length IgG1 heavy chain variant 2)
SEQ ID NO: 16
QVQLVQSGAEVKKPGATVKISCKVSGYTFTDYYMHWVQQAPGKGLEWMGLVDPED
GETIYAEKFQGRVTITADTSTDTAYMELSSLRSEDTAVYYCATDARGSGSYYPNH
FDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALH
NHYTQKSLSLSPGK
(Human full length IgG1 light chain variant 2)
SEQ ID NO: 17
DIVMTQTPDSLAVSLGERATINCKSSQSVLYSSNNKNYLAWYQQKPGQPPKLLIY
WASTRESGVPDRFSGSGSGTDFTLTINSLQAEDVAVYFCQQYYDTPTFGQGTRLE
IKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSENRGEC
(Fab heavy chain variant 1)
SEQ ID NO: 18
QVQLVESGAEVKKPGATVKISCKVSGYTFTDYYMHWVQQAPGKGLEWMGLVDPED
GETIYAEKFQGRVTITADTSTDTAYMELSSLRSEDTAVYYCATDARGSGSYYPNH
FDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDK
KVEPKSC
(Fab light chain variant 1)
SEQ ID NO: 19
DIVMTQSPDSLAVSLGERATINCKSSQSVLYSSNNKNYLAWYQQKPGQPPKLLIY
WASTRESGVPDRESGSGSGTDFTLTINSLQAEDVAVYFCQQYYDTPTFGQGTRLE
IKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
(Human truncated Gremlin-1 used in crystallography
without N-terminal tag)
SEQ ID NO: 20
AMPGEEVLESSQEALHVTERKYLKRDWCKTQPLKQTIHEEGCNSRTIINRFCYGQ
CNSFYIPRHIRKEEGSFQSCSFCKPKKFTTMMVTLNCPELQPPTKKKRVTRVKQC
RCISIDLD
(Mature Gremlin-1 sequence of SEQ ID NO: 1
lacking the signal peptide of amino acids 1-21)
SEQ ID NO: 21
KKKGSQGAIPPPDKAQHNDSEQTQSPQQPGSRNRGRGQGRGTAMPGEEVLESSQE
ALHVTERKYLKRDWCKTQPLKQTIHEEGCNSRTIINRFCYGQCNSFYIPRHIRKE
EGSFQSCSFCKPKKFTTMMVTLNCPELQPPTKKKRVTRVKQCRCISIDLD
(Human IgG4P heavy chain variant 1)
SEQ ID NO: 22
QVQLVESGAEVKKPGATVKISCKVSGYTFTDYYMHWVQQAPGKGLEWMGLVDPED
GETIYAEKFQGRVTITADTSTDTAYMELSSLRSEDTAVYYCATDARGSGSYYPNH
FDYWGQGTLVTVSSASTKGPSVFPLAPCSRSTSESTAALGCLVKDYFPEPVTVSW
NSGALTSGVHTFPAVLQSSGLYSLSSVVTVPSSSLGTKTYTCNVDHKPSNTKVDK
RVESKYGPPCPPCPAPEFLGGPSVFLFPPKPKDTLMISRTPEVTCVVVDVSQEDP
EVQFNWYVDGVEVHNAKTKPREEQFNSTYRVVSVLTVLHQDWLNGKEYKCKVSNK
GLPSSIEKTISKAKGQPREPQVYTLPPSQEEMTKNQVSLTCLVKGFYPSDIAVEW
ESNGQPENNYKTTPPVLDSDGSFFLYSRLTVDKSRWQEGNVFSCSVMHEALHNHY
TQKSLSLSLGK
(Human IgG4P light chain variant 1)
SEQ ID NO: 23
DIVMTQSPDSLAVSLGERATINCKSSQSVLYSSNNKNYLAWYQQKPGQPPKLLIY
WASTRESGVPDRFSGSGSGTDFTLTINSLQAEDVAVYFCQQYYDTPTFGQGTRLE
IKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
(Human IgG1 heavy chain DNA variant 1)
SEQ ID NO: 24
caagtgcaactggtggaatccggggccgaagtgaaaaagcccggagccactgtga
agatctcttgcaaagtgtccggctacaccttcaccgactattacatgcactgggt
ccagcaggcacctgggaagggccttgagtggatgggtctggtcgatcccgaggac
ggcgaaactatctacgccgagaagttccagggtcgcgtcaccatcaccgccgaca
cttccaccgacaccgcgtacatggagctgtccagcttgaggtccgaggacacagc
cgtgtactactgcgccacggatgctcggggaagcggcagctactacccgaaccac
ttcgactactggggacagggcactctcgtgactgtctcgagcgcttctacaaagg
gcccctccgtgttcccgctcgctccatcatcgaagtctaccagcggaggcactgc
ggctctcggttgcctcgtgaaggactacttcccggagccggtgaccgtgtcgtgg
aacagcggagccctgaccagcggggtgcacacctttccggccgtcttgcagtcaa
gcggcctttactccctgtcatcagtggtgactgtcccgtccagctcattgggaac
ccaaacctacatctgcaatgtgaatcacaaacctagcaacaccaaggttgacaag
aaagtcgagcccaaatcgtgtgacaagactcacacttgtccgccgtgcccggcac
ccgaactgctgggaggtcccagcgtctttctgttccctccaaagccgaaagacac
gctgatgatctcccgcaccccggaggtcacttgcgtggtcgtggacgtgtcacat
gaggacccagaggtgaagttcaattggtacgtggatggcgtcgaagtccacaatg
ccaaaactaagcccagagaagaacagtacaattcgacctaccgcgtcgtgtccgt
gctcacggtgttgcatcaggattggctgaacgggaaggaatacaagtgcaaagtg
tccaacaaggcgctgccggcaccgatcgagaaaactatctccaaagcgaagggac
agcctagggaacctcaagtctacacgctgccaccatcacgggatgaactgactaa
gaatcaagtctcactgacttgtctggtgaaggggttttaccctagcgacattgcc
gtggagtgggaatccaacggccagccagagaacaactacaagactacccctccag
tgctcgactcggatggatcgttcttcctttactcgaagctcaccgtggataagtc
ccggtggcagcagggaaacgtgttctcctgctcggtgatgcatgaagccctccat
aaccactatacccaaaagtcgctgtccctgtcgccgggaaag
(Human IgG1 light chain DNA variant 1)
SEQ ID NO: 25
gacattgtgatgacccagtcccccgattcgcttgcggtgtccctgggagaacggg
ccaccattaactgcaagagctcacagtccgtcctgtattcatcgaacaacaagaa
ttacctcgcatggtatcagcagaagcctggacagcctcccaagctgctcatctac
tgggctagcacccgcgaatccggggtgccggatagattctccggatcgggttcgg
gcactgacttcactctgactatcaactcactgcaagccgaggatgtcgcggtgta
cttctgtcagcagtactacgacaccccgacctttggacaaggcaccagactggag
attaagcgtacggtggccgctccctccgtgttcatcttcccaccctccgacgagc
agctgaagtccggcaccgcctccgtcgtgtgcctgctgaacaacttctacccccg
cgaggccaaggtgcagtggaaggtggacaacgccctgcagtccggcaactcccag
gaatccgtcaccgagcaggactccaaggacagcacctactccctgtcctccaccc
tgaccctgtccaaggccgactacgagaagcacaaggtgtacgcctgcgaagtgac
ccaccagggcctgtccagccccgtgaccaagtccttcaaccggggcgagtgc
(Human IgG4P heavy chain DNA variant 1)
SEQ ID NO: 26
caagtgcaactggtggaatccggggccgaagtgaaaaagcccggagccactgtga
agatctcttgcaaagtgtccggctacaccttcaccgactattacatgcactgggt
ccagcaggcacctgggaagggccttgagtggatgggtctggtcgatcccgaggac
ggcgaaactatctacgccgagaagttccagggtcgcgtcaccatcaccgccgaca
cttccaccgacaccgcgtacatggagctgtccagcttgaggtccgaggacacagc
cgtgtactactgcgccacggatgctcggggaagcggcagctactacccgaaccac
ttcgactactggggacagggcactctcgtgactgtctcgagcgcttctacaaagg
gcccctccgtgttccctctggccccttgctcccggtccacctccgagtctaccgc
cgctctgggctgcctggtcaaggactacttccccgagcccgtgacagtgtcctgg
aactctggcgccctgacctccggcgtgcacaccttccctgccgtgctgcagtcct
ccggcctgtactccctgtcctccgtcgtgaccgtgccctcctccagcctgggcac
caagacctacacctgtaacgtggaccacaagccctccaacaccaaggtggacaag
cgggtggaatctaagtacggccctccctgccccccctgccctgcccctgaatttc
tgggggaccttccgtgttcctgttccccccaaagcccaaggacaccctgatgatc
tcccggacccccgaagtgacctgcgtggtggtggacgtgtcccaggaagatcccg
aggtccagttcaattggtacgtggacggcgtggaagtgcacaatgccaagaccaa
gcccagagaggaacagttcaactccacctaccgggtggtgtccgtgctgaccgtg
ctgcaccaggactggctgaacggcaaagagtacaagtgcaaggtgtccaacaagg
gcctgccctccagcatcgaaaagaccatctccaaggccaagggccagccccgcga
gccccaggtgtacaccctgccccctagccaggaagagatgaccaagaaccaggtg
tccctgacctgtctggtcaagggcttctacccctccgacattgccgtggaatggg
agtccaacggccagcccgagaacaactacaagaccaccccccctgtgctggacag
cgacggctccttcttcctgtactctcggctgaccgtggacaagtcccggtggcag
gaaggcaacgtcttctcctgctccgtgatgcacgaggccctgcacaaccactaca
cccagaagtccctgtccctgagcctgggcaag
(Human IgG4P light chain DNA variant 1)
SEQ ID NO: 27
gacattgtgatgacccagtcccccgattcgcttgcggtgtccctgggagaacggg
ccaccattaactgcaagagctcacagtccgtcctgtattcatcgaacaacaagaa
ttacctcgcatggtatcagcagaagcctggacagcctcccaagctgctcatctac
tgggctagcacccgcgaatccggggtgccggatagattctccggatcgggttcgg
gcactgacttcactctgactatcaactcactgcaagccgaggatgtcgcggtgta
cttctgtcagcagtactacgacaccccgacctttggacaaggcaccagactggag
attaagcgtacggtggccgctccctccgtgttcatcttcccaccctccgacgagc
agctgaagtccggcaccgcctccgtcgtgtgcctgctgaacaacttctacccccg
cgaggccaaggtgcagtggaaggtggacaacgccctgcagtccggcaactcccag
gaatccgtcaccgagcaggactccaaggacagcacctactccctgtcctccaccc
tgaccctgtccaaggccgactacgagaagcacaaggtgtacgcctgcgaagtgac
ccaccagggcctgtccagccccgtgaccaagtccttcaaccggggcgagtgc
(Mouse full length IgG1 heavy chain variant 2)
SEQ ID NO: 28
QVQLVQSGAEVKKPGATVKISCKVSGYTFTDYYMHWVQQAPGKGLEWMGLVDPED
GETIYAEKFQGRVTITADTSTDTAYMELSSLRSEDTAVYYCATDARGSGSYYPNH
FDYWGQGTLVTVSSAKTTPPSVYPLAPGSAAQTNSMVTLGCLVKGYFPEPVTVTW
NSGSLSSGVHTFPAVLQSDLYTLSSSVTVPSSTWPSETVTCNVAHPASSTKVDKK
IVPRDCGCKPCICTVPEVSSVFIFPPKPKDVLTITLTPKVTCVVVDISKDDPEVQ
FSWFVDDVEVHTAQTQPREEQFNSTFRSVSELPIMHQDWLNGKEFKCRVNSAAFP
APIEKTISKTKGRPKAPQVYTIPPPKEQMAKDKVSLTCMITDFFPEDITVEWQWN
GQPAENYKNTQPIMDTDGSYFVYSKLNVQKSNWEAGNTFTCSVLHEGLHNHHTEK
SLSHSPGK
(Mouse full length IgG1 light chain variant 2)
SEQ ID NO: 29
DIVMTQTPDSLAVSLGERATINCKSSQSVLYSSNNKNYLAWYQQKPGQPPKLLIY
WASTRESGVPDRFSGSGSGTDFTLTINSLQAEDVAVYFCQQYYDTPTFGQGTRLE
IKRTDAAPTVSIFPPSSEQLTSGGASVVCFLNNFYPKDINVKWKIDGSERQNGVL
NSWTDQDSKDSTYSMSSTLTLTKDEYERHNSYTCEATHKTSTSPIVKSFNRNEC
(Human full length IgG1 heavy chain variant 1)
SEQ ID NO: 30
QVQLVESGAEVKKPGATVKISCKVSGYTFTDYYMHWVQQAPGKGLEWMGLVDPED
GETIYAEKFQGRVTITADTSTDTAYMELSSLRSEDTAVYYCATDARGSGSYYPNH
FDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALH
NHYTQKSLSLSPGK
(Human full length IgG1 light chain variant 1)
SEQ ID NO: 31
DIVMTQSPDSLAVSLGERATINCKSSQSVLYSSNNKNYLAWYQQKPGQPPKLLIY
WASTRESGVPDRESGSGSGTDFTLTINSLQAEDVAVYFCQQYYDTPTFGQGTRLE
IKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
(Fab heavy chain variant 2)
SEQ ID NO: 32
QVQLVQSGAEVKKPGATVKISCKVSGYTFTDYYMHWVQQAPGKGLEWMGLVDPED
GETIYAEKFQGRVTITADTSTDTAYMELSSLRSEDTAVYYCATDARGSGSYYPNH
FDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDK
KVEPKSC
(Fab light chain variant 2)
SEQ ID NO: 33
DIVMTQTPDSLAVSLGERATINCKSSQSVLYSSNNKNYLAWYQQKPGQPPKLLIY
WASTRESGVPDRFSGSGSGTDFTLTINSLQAEDVAVYFCQQYYDTPTFGQGTRLE
IKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
(Human IgG4P heavy chain variant 2)
SEQ ID NO: 34
QVQLVQSGAEVKKPGATVKISCKVSGYTFTDYYMHWVQQAPGKGLEWMGLVDPED
GETIYAEKFQGRVTITADTSTDTAYMELSSLRSEDTAVYYCATDARGSGSYYPNH
FDYWGQGTLVTVSSASTKGPSVFPLAPCSRSTSESTAALGCLVKDYFPEPVTVSW
NSGALTSGVHTFPAVLQSSGLYSLSSVVTVPSSSLGTKTYTCNVDHKPSNTKVDK
RVESKYGPPCPPCPAPEFLGGPSVFLFPPKPKDTLMISRTPEVTCVVVDVSQEDP
EVQFNWYVDGVEVHNAKTKPREEQFNSTYRVVSVLTVLHQDWLNGKEYKCKVSNK
GLPSSIEKTISKAKGQPREPQVYTLPPSQEEMTKNQVSLTCLVKGFYPSDIAVEW
ESNGQPENNYKTTPPVLDSDGSFFLYSRLTVDKSRWQEGNVFSCSVMHEALHNHY
TQKSLSLSLGK
(Human IgG4P light chain variant 2)
SEQ ID NO: 35
DIVMTQTPDSLAVSLGERATINCKSSQSVLYSSNNKNYLAWYQQKPGQPPKLLIY
WASTRESGVPDRFSGSGSGTDFTLTINSLQAEDVAVYFCQQYYDTPTFGQGTRLE
IKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
(Human Gremlin-1; full sequence)
SEQ ID NO: 36
actcggtgcgccttccgcggaccgggcgacccagtgcacggccgccgcgtcactc
tcggtcccgctgaccccgcgccgagccccggcggctctggccgcggccgcactca
gcgccacgcgtcgaaagcgcaggccccgaggacccgccgcactgacaggtgagcg
cggacgcacccggcagggatgtgagtgggcggagggaagagggccgcaaaccaac
ccaggacccgctcagttccacgcgcggcagccctccgtgcgcgcaggctcgggtg
cgttgttcgcgggggtgaattgtgaagaaccatcgcggggtccttcctgctgagg
ccgcggacaccgtgacctcgctgctctgggtctgcagggaaacgtaggaaaaaaa
gttgtcaggagcgggcaggatgacccccacatcccgtttccacctcccggaggcc
cccgaacacgctcctggtgctggtggcagcagcgcctggcagacgcgcccgctta
gcgagggcgcgaagtccaggccgccagagcgcaggagcatccggacctgctagtc
ggccgctgactgcgcggcgagttgccttgagagggtcccatgtgcttggggcgcc
gcgctgggtctgggggcgtcttggggcgcccattggagtccgcgggttggagcat
ccggagaatccatgatgtgtgcatttgccgatccccgaggtgagatggagactgg
caagggcagagccgctgtgttcagccacagcggaaaaccgaacggtgggtaatcc
gacagctgcggtgcggggcgcggccctggccgcggggtccagcgaacccgcagtg
ctcacaaggcagacaccacacgcgctcgcggaccggccacgcactcgcgggcgct
cgcttctctactccagcctcttccccgccccgcgcacgcccgagctgaatggtag
acgttctggcgccgggcagcggccaccggctggttcccacttccgcgcgcacccc
ttaaactgtgttctagaggccccagcctcgccttgcagcgcctcactagctcctg
aggactagggactggcggctgaggcgggttggcggctgcaacgagctgggcgtct
ttcgttctctctcgctgcctggctggctccgctggcccctccacagcttgcggag
caaggccatagcaggggagtgggaggtatattggggctgtcacctccttgctggc
cggagttatttgtagactacagactccggaagaacagacgcgccaccgctctcgc
ttggcattgccttcggatcgcagctcctccttgggggtgccccagcttggcgttt
atttgcctgcgccaggctctggcgacggtcaccgggccaggcggggagggacgga
cggcaggtgaccagcctctgctgtgaagaaattcctgcgcgcccggagctgtccc
taatgcattcccgggtcgaatccgtctactgccttcccctcctcgaccgactccg
aatctcggctcttatagacagaaatacagcctcagcgttaggggttaaaatcccc
ctcttaaacggtccgagggcagagaggtgaccaccgataggtaattggatctcct
gctggaaagagcaaatctgagcggtgtgcgcgtctgtttatgttccccttcgaga
tggtgccaggacacgaactgattaaaacaatctattgtgttaagtgggtcactag
ggttttaagctgtcccagggaccccagagtagtggcttccttctggctgtacaca
caagttaaataaatagcgtagaagaggttaagataaccccattctagggtgagga
gtcctctttcatccctagggcttccccctccccttttctctttttttggaaggag
ggggagcatgagagtcttgagggggggatgtacttttcaaagcaaggagggaaag
atcttaagaaaactatatattctcactgccccccaagccaagtctataacagtag
gtgatttgattactatctctggataaatggcactgtcaaattgttaatattaact
atttcagggatttttagcagggtagtggcagtatgtgtgcgtgtgtgtgtgtgtg
tctgtgtgtgtgtgtttaacctccaggtcattgtaggaattagagtcttttgtaa
actttgtaatttcacaggtttcctattttcttaaaagttcatttttagtgaaatg
ttttggtaacccacgctctgtaggaaatccaggttggctaatgcggtctttatgt
gagtagttacacagggaaggataaaaaccttttatgtcctacatctctgaatgag
ggctgcctaccctgtctttgaaactaagccgaagatgccttcagtctgaatggtc
aagtattaaaagtgataaaatgcaaagaaatttcatgccgcagacacctccccca
agaactgcttgttgacagcaaagctgtggaacatgttccacaacagagagtaaag
gacagccaggaaatataaaccttttatgtaaaggaaaggcaggtgggggacagtg
gttaggggaggtgactgcagcctctaaccaaaaggcaaccatcaggcaagtgcta
ccagcccgtgtcttcgatctgcaaggaattttctttagttttaacatatgctctt
agaaattcaaagtacaacaggaattcctgggacaagagaaatctttttattcaca
tgtgaacatgaagatacaaaatagataattattttatttatagcactcttcaaat
tgtattgcattagaaaacatatccattgacccactgttaaggacagcactgggtg
tcaataggacagtggttaaggacctgtgtttggggctagatagaattgggtttaa
actgctggctgggctgggcacagtggctcacaccggtaatcccagcactttggga
ggccaaggaggacggatcacctgaggtcgggagttcgacaccagcctgaccaaca
tggaaaaatcccgtctctactaaaaacacaaaattagccaggcatggtggtgcat
gcctgcaatcccagctacttgggaggctgaggcaggagaattgcttgaaaccggg
aggcggaggttgtggtgagcccagatagcgccattgcattccagcctgggcaaca
cgagtgaaaactccgtcaaaaaaacaaaacaaaacaaacaaacaaaaaaatgctg
gctttcccacttatgagctgtgtgaccttggacaaatttccaacttttctgagtg
tagattccctgattggtaaaaggaagatgatattatctacctcatattttgttat
gaaaaataaatgataaaattgggtcagaaatcagcataatgcctggcacagtaag
ggcttcaaaataaaaggtagctcttattattagtaatggtgttaggaaaagtagc
aatgttatacagaaccaggatatatcacagggcagttctgaaattaaatcctgaa
tcctggccgggtgaggtggctcacgcttgtaatcctaagcactttcggagactga
ggcaggcggatcacgaggtcaggagtttgagaccagcctggccaacacactgaaa
tcccgtctctactaaaaatacaaaaattagctgggtgtggtggcaggcgcctata
atctcagctacttgggaggctgaggcaggagaatcacttgagcccaggaggcgga
ggttgcagtgagctgatatcgtgccagtgcactccagcctgggtgacatctctta
aaaaaaaaaatcctgaataccacactacgcagtgactaacacatctttcactaca
gaacagaacctgtaacttggccgtctctcagcagtgctgctcagtgaacatttaa
taatttattactttctaactcgtttcttgttgacctcaagaattgtacatagtca
ttaactttcctaagaaaatctttgacaaacatagagctcctgagatatttcacaa
ccaggtggtctcctccctgtcctatgcaatgttgggccccagcctgatttagccg
acctggtcttcagacttgagaggctgtttagggttcttacaacacaaaggggatg
agactttatcctctacctgtgtgccaacaagggattcttcttatctccttggtgc
aacttgtctgaaaaagaaagtcaacacaattatcttcttaaaagttaaagatcaa
attaaaaataagctatagttttcccaaagatttagacctgagaaaaaggaataga
tctttctaaaacctggcctgcacttagagcatttgtagtcacttccactatttct
tatgctgagagaataatttgatgtcatgcctattgaatgtctttctaaagcttga
ttcatcaggaggaactgaccagaagtccatgcacagacatttggctttcactgta
attcctactcaaaactccctttcactgattatgagcaggttgcttagctaaaatg
agaaatacagcaagaagagggatggaaagagctggcttaaactttcccaaagaca
tcatgaacactgggaacaggtcatacatataccatttttatttctcagtcctcag
tattagaatattgtgttcctagaggctttgtgaaaattggaatacattgccatga
cctgcacaggatagaggtggttaatatggcttaccttgcttgtagatgtttactc
ttgatccctaagggaaactggaaggaaagcatgctgtagagaggacctgctttga
gagtatgtgtcctctgggacaagtggaatgaaagagaggatagaggcaaagagaa
aaaaaggtggagatggggtgagacttgctggtggacagttgcaaagaaaactcat
gatgagaataacatcacatttcttgaagaagctgaactgcattgatgggaagttg
aggcagagtttgaaaagaagatatactgtctgcaaaaaagaatcaacggagatgt
caggtatcttgatgcaccctgagtctatgagatgcagcttaatttaatttaatga
tatgagtaggccatttcataagtggtggaaattaccataaagtgtttgcagctaa
tgacggcgcatgaatgatgtcataggaacctaagtctggatgagctatggattga
atttttactatgggacacccttctacatgttgctgtggaaaagaattgtcctcaa
gaaatgtacatcttctgcatttcctctacatctctgatatttacaaagtgcacat
tatttggtgatactacaactgggatttcaagtgcacatcactactatttccttca
ctgaagaatcaagagcagtctgggggggggagagcttgagtgattgacaaggatg
tggcagagccctggttccactatgaattatagttcttaccctgcttactgtcact
catgtagagcagctagtgtgcctcagagcactgctgttctctgagaagctgaggt
ctcgattgacattcttgaagttggtgtttacttctctctgaataaacaaaggttg
gcaactcagacatcttacaataagtactaaagatttttgaagaataggtttttaa
aaaatgaaaaaacatttcactttccatagctaataaatcttattttgaggaaaat
gtacttttctttaaaaaaaaaaaaagcctgtctgtcactctagaccctttggctt
agaaggtaggcacactcacatagaaacagaaagtctgtccaaattaaaactgaaa
accacagttgactaattttgaatttatagctctgctgttggcttctgcgatagta
ttaatttcaatggcttcaattagaaaatgaaacccatagcattccatatgagaac
aggtaaaaagtcagggacatttggagttttccaagaaaaagaaagacaagtctta
ggaagctctctaggatggaaggaatttgccacactgagagttagacatccaaagg
atagcaattggctcttctgctcatgggcactggtgaaggcattttaaaatgcgaa
gaatggtacctctgtaaatcaatgaggttcataataatcatgcatttaccaaatt
tttataagcacctgccttgtgccaggcactgagggtaaggtgatgaataagccct
catcaactgtcagactagatatttactcaataacagatgtgaaaatgccaagaag
gaaaagttgagtataaagaaagccttaagttggttcagagaaataaaattgcatt
tttcggatagatgtttatggaatcggccttatgaggtaaacttgtcctatgcagt
gaacatacattcccagttagtctcagattggcctctgtgatgacaaactcagagg
gtcctggtctaggaggggtgaatttgtctggaggccattttcaggaggtatggag
gaagactggggcataggcctggggccatcccatgtactcctcctctgaaatgggg
agcaactgaattgtgttttattttagatcttcgtccaacttgaataccagaaatt
cgtgaaaccttctcaaattcacactatattttgagaccaggagaaggctccttga
gaaattgccacactgtcttatcctagtctctggaaaaattcagtcctgtattata
actgggcgtttctcataagtgctttttttttttctttttctttttataatacttt
aagttctgggatacatgtgcagaatgtgcaggtttgttacataggtatacacgtg
ccatggtggtttgctgcacccatcaacccatcatctacattaggtatttctccta
atgctaccccttccctagccccccacccctcgacaggccccagtgtttgatgttc
ccctccctgtgtccatgtgttctcattgttcaactcccacttatgagtgagagca
tgcggtgtttggttttctgttcctgtgttagtttgctgagaatgatggtttttgg
cttcatccacgtccctgcaaaggacatgaactcatcctttttatggctgcatagt
attccatggtgtatatgtgccacatttgctttatccagtctatcattgatgagca
cttgggttggctccaagtctttgctattatgactagtactgcgataaacatgtgt
atatgtgtctttatattagaatgatttataatcctttaggtctatacccagtaat
aggattcctgggtcaaatggtatttctggttctagatccttgaggaattgccaca
ctgtcttccacaacggttgaactaatttacactcccaccaacagtgtaaaagcat
tcctatttctccacatcctctccaacatctgttgtttcctgactctttaatgacg
gccattctaactggcgtgaaatggtatctcattgtggttttgatttgcatttctc
taatgaccaccgatgatgagctttttctcatatgtttgctggccccataaatgtc
ttcttttgagaaatgtctgttcatatcctttgcccactttttgatggggttgttt
gcttttttcttgtaaatttgtttgagttcctggtagattctggatattagccctt
tgtcagatggatagactgcaaaaattttctcccattctgtaggttgcctgttcac
tctgatggtagtttcttttgccatgcagaagctgtttagtttaattagatccaat
ttgtcagttttggcttttgttgccattgcttttggtgttttagtcatgaagtctt
tgcccatgcctatgtcctgaattgtattgccctggttttcttctaggatttttat
ggttttaggtcttacatttaagtctttaatccatcttgaattaatttttgtataa
ggtgtaaggaaagggtccagtttcagttttctgcgtatggctagccagttttccc
aacgttatttattaaatagggaatcctttccccattgcttgtttttgtcaggttt
gtcaaagatcagacggttgtagatgtgtggtgttatttctgaggcctctgttctg
ttccattggtctgtatatctgctttgataccagtaccgtgctgttttggttactg
tagccttgtagtacagtttgaagtcaggtagcgtgatgctttgttctttttgctt
aggattgtcttggctatatgggctcttttttggttctatatgaaatttaaagtag
ttttttcatagacatctatagaactctccaccccaaatcaacagaatatacattc
ttctcagtatcgcatcacacttattctaaaatgaccacataattagaagtaaaac
actcctcagcaaatgcaaaaaacagaaatcctaacagtctgtcagaccccagtgc
aatcaaattagaactcaggattaagaaacttactgaaaaccacacaactgcatgg
aaactgaacaacctgctcctgaatgactactgggtaaataacaaaattaaggcag
aaataaataggttctttgaaagaaatgagaacaaagacacaatataccagaatct
ctgggacacagctaaagcagtgtttagagggaaattgatagcactaaatgcccac
aggagaaagcgggacagatctaaaatcgacaccctaatatcacaattaaaagaac
tagagaaacaagagcaaacaaattcaaaagctagcagaagacaagaaataactaa
gatcagagcagaactgaaggagatagagacacaaaaaacccttcaaaaaatcaat
gaatccaggagctgtttttttttttttgaaaagattaatgaaatagactgctagc
caaactaataaaggagaaaagagagaagaatcaaatagacaatagacacaataaa
aagtgataaaggggatatcaccactgatcccacagaaatacaaactaccatcaga
gaatactataaacacctctacgcaaataaactagaaaattgggaagaaatggata
cattcctgaacgcatacaccctcccaagactaaaccaggaagaagttgaatccct
gaatagaccaatagcaagttctgaaattgaggcagtaattagtagcctaccaacc
aaaaaaagcccaggaccagacagattcacagccgaagtctaccagaggtacaaag
aggagctagtaccattccttctgaaactattccaaacaatagaaaaagaaggact
cctccctaactcattttatgaggccagcatcatcctgataccaaaacctggcaga
gacacaacataaaggaaattcaggccaatatccctgatgaacattgatgtgaaaa
tcctcaataaaatactggcagcacatcaaaaagcttacctgccatgatcaagttg
gcttcatccctgggatgcaaggctggttcaacatacgcaaatcaataaacgtaat
caatcacatacacagaaccaatgacaaaaaccacatgattatctcaatagatgca
gaaaaggccttcgataaaattcaacaccccttcaatgctaagaactctcaataaa
ctaggtattgatgaaacatatctcaaaataataagagttatttatgacaaaccca
cagccagtatcatactgaatgggcaaaagctggaagcattccctttgaaaaccgc
cacaaggatgacttctctcaccactcctattcaacatactattggaagttctggc
cagggcaagcaggcaagacaaataaataaagggtattcaaataggaagagaggaa
gtcaaattgtctctgtttgcagatgacatgattgtatatttagaaaaccccatca
tctcagcccaaaatctccttaagctgataagcaacttcagcaaagtctcaggata
caaaatcaatgtgcaaaaatcacaagcattcctatacaccaataatagacaaaca
gccaaatcatgagtgaactcccattcacaagtgctacaaagaataaaatacctag
gaatccaacttacaagggatgtgaaggacctcttcaaggagaactacagaccact
gctcaaggtaataagagaggacacaaacaaatggaaaaacattccatgctcatgg
ataggaagaatcaatattatgaaaatggccatactgcccaaaacaatttatagat
tcaatgccatccccatcaagctaccactgacttccttcacagaattagaaaaaaa
ctactttaaatttcatatggaactgaaaaaagagccggtatagccaagacgatcc
taagcaaaaagaaaaaagctggaagcatcatgctacctgacttcaaactatacta
caaggctacagtaacaaaaacagcatggtatagtataagtgcttttttttaaaag
acaaagtaaagtaatttttttgttgttggggtaaaacaaaagctctgcataaaga
gcagggatgttgtaacatacactgaccaaaggtgggaaacctacagttggagcag
aagctgaatgtcacattatcagctccgaacttataatggtctaaaagtactaggt
taatgttggaaagatggtgccatttaaagatatcttaaattcaatatttaattat
tttaatttgacattatcctagagttgaatggtgtttacttaccatgtgctgttca
ttagaaaatctagatcctacactgcctttgcgcaaggtagttgctctaataatac
caacctgtccagttttggtgggagaaataatgttactgttaagttgcacttagtg
gttattatgtgtaatactggtattccaaagagagaaggaaaatgtttgctacaca
gctgtgttcttaggttcagaaaaccacaggagtgggacaggagaaccttcaggat
tcaggtccgattgttgtgatggccgcaggagggagactgtgaatttgaaactgca
tccattgaaaagaaaatccctccacactttaataattctctccgtgccccatggc
agcaagtgcttataggccttgctactcaaagcttaataaaaaggcagacctgctg
aatcataatctgcatcttaacaatatccccagatattgtctgcactttttttttt
ttttctttctttctttcttcttttttttgagacagagtctcgctcagtcacccag
gctggagtgcagtggcgcgatctcggctcactgcaagctctgactcccgggttca
cgccattctcctgccttagactcccgagtagctggcactacaggcgcccgccact
acgcccggctaatttttttgtatttttagtagagatggggtttcaccgtgttagc
caggatggtctcgatctcctgacctcgtgatccgccgcccatctgggcctcccaa
agtgctgggattacaggcgtgagccactgcgcccggccaattgtctgcactttca
agtctaagaagcactgtcccaggaggaaaatctttttagtctgaggcttctctct
tgcactctcctttttaaaaatatgctgctccttctccacctttccctcttcttcc
gccttttctgtctgcctttactacctcccctgaacattcaacttgtagaagagtt
cccctttctctgaattgcattcttcacttcattcattcttttctctctctgtcta
tggtttcctattttttgtcggttttccctcacctcacctccttgttattttttgc
cattgttcacatatcactgccctttcagacccatatctagcttctgacccatcca
ctaatccattgccactaatttattcagcatgcccatgccatttattaatgtaaat
atttgcacatacttgttctatcatgcccatttttctaccttttaaattgtatata
cacagacacatgtgaatgacatatttcactatttaaaaggtagaaaaatgtatat
cgtggtacaaagtgatacattgagtatctgtgcccagtttcaagatgagaataag
tgagaggtgaagcctcatgagtcacgtcacctacacgctactgtctttcattcca
tctgcagtactgccacacatttggttagttctccaattgctgtcacattgacatc
gagttggatctgaacagcgtacctggggagacgaagtattggtatctttgcttaa
atggagtgatttactgagagacaaagtgacatctttaagatcagatagcaagtta
ctgcaccaggagcgcgacctttcctgttttgttgttttccctctgtcaaatgcct
tctggtctccatgaatctctccttccacatatgataaaaatgcaaagtctcataa
gcattcacagctcaggaagcctcaggctagttggggagaaaagactgggaggttt
ccggaggaatgaagtcctctgagcagagaggttaattcatcttgctgtaaaacaa
aatagaaaataagttcccctcaataagtgaacgtaatacaaagacaaatgtggtt
ggtgccagaggccaggaagaagttcttgtgagaataggtgcagagaagagctagg
ccctggctgagtttaaaccttgacttagtcactgtgggactctgggtgagttact
ccatggattgatggctgggtcatggagataatagtacctaattcatacaggtact
gtgagaagtaaatggaatatttcacgttaagtgtttaacggtgcgtttaaatgct
aggtgctattattattaatttttaaattaactttgccatgttttgtgtcttcccc
tctctgtgcttcctttctttagtatgagccgcacagcctacacggtgggagccct
gcttctcctcttggggaccctgctgccggctgctgaagggaaaaagaaagggtcc
caaggtgccatccccccgccagacaaggcccagcacaatgactcagagcagactc
agtcgccccagcagcctggctccaggaaccgggggggggccaagggcggggcact
gccatgcccggggaggaggtgctggagtccagccaagaggccctgcatgtgacgg
agcgcaaatacctgaagcgagactggtgcaaaacccagccgcttaagcagaccat
ccacgaggaaggctgcaacagtcgcaccatcatcaaccgcttctgttacggccag
tgcaactctttctacatccccaggcacatccggaaggaggaaggttcctttcagt
cctgctccttctgcaagcccaagaaattcactaccatgatggtcacactcaactg
ccctgaactacagccacctaccaagaagaagagagtcacacgtgtgaagcagtgt
cgttgcatatccatcgatttggattaagccaaatccaggtgcacccagcatgtcc
taggaatgcagccccaggaagtcccagacctaaaacaaccagattcttacttggc
ttaaacctagaggccagaagaacccccagctgcctcctggcaggagcctgcttgt
gcgtagttcgtgtgcatgagtgtggatgggtgcctgtgggtgtttttagacacca
gagaaaacacagtctctgctagagagcactccctattttgtaaacatatctgctt
taatggggatgtaccagaaacccacctcaccccggctcacatctaaagggggggg
ccgtggtctggttctgactttgtgtttttgtgccctcctggggaccagaatctcc
tttcggaatgaatgttcatggaagaggctcctctgagggcaagagacctgtttta
gtgctgcattcgacatggaaaagtccttttaacctgtgcttgcatcctcctttcc
tcctcctcctcacaatccatctcttcttaagttgatagtgactatgtcagtctaa
tctcttgtttgccaaggttcctaaattaattcacttaaccatgatgcaaatgttt
ttcattttgtgaagaccctccagactctgggagaggctggtgtgggcaaggacaa
gcaggatagtggagtgagaaagggagggtggagggtgaggccaaatcaggtccag
caaaagtcagtagggacattgcagaagcttgaaaggccaataccagaacacaggc
tgatgcttctgagaaagtcttttcctagtatttaacagaacccaagtgaacagag
gagaaatgagattgccagaaagtgattaactttggccgttgcaatctgctcaaac
ctaacaccaaactgaaaacataaatactgaccactcctatgttcggacccaagca
agttagctaaaccaaaccaactcctctgctttgtccctcaggtggaaaagagagg
tagtttagaactctctgcataggggtgggaattaatcaaaaaccgcagaggctga
aattcctaatacctttcctttatcgtggttatagtcagctcatttccattccact
atttcccataatgcttctgagagccactaacttgattgataaagatcctgcctct
gctgagtgtacctgacagtagtctaagatgagagagtttagggactactctgttt
tagcaagagatattttgggggtctttttgttttaactattgtcaggagattgggc
taaagagaagacgacgagagtaaggaaataaagggaattgcctctggctagagag
tagttaggtgttaatacctggtagagatgtaagggatatgacctccctttcttta
tgtgctcactgaggatctgaggggaccctgttaggagagcatagcatcatgatgt
attagctgttcatctgctactggttggatggacataactattgtaactattcagt
atttactggtaggcactgtcctctgattaaacttggcctactggcaatggctact
taggattgatctaagggccaaagtgcagggtgggtgaactttattgtactttgga
tttggttaacctgttttcttcaagcctgaggttttatatacaaactccctgaata
ctctttttgccttgtatcttctcagcctcctagccaagtcctatgtaatatggaa
aacaaacactgcagacttgagattcagttgccgatcaaggctctggcattcagag
aacccttgcaactcgagaagctgtttttatttcgtttttgttttgatccagtgct
ctcccatctaacaactaaacaggagccatttcaaggcgggagatattttaaacac
ccaaaatgttgggtctgattttcaaacttttaaactcactactgatgattctcac
gctaggcgaatttgtccaaacacatagtgtgtgtgttttgtatacactgtatgac
cccaccccaaatctttgtattgtccacattctccaacaataaagcacagagtgga
tttaattaagcacacaaatgctaaggcagaattttgagggtgggagagaagaaaa
gggaaagaagctgaaaatgtaaaaccacaccagggaggaaaaatgacattcagaa
ccagcaaacactgaatttctcttgttgttttaactctgccacaagaatgcaattt
cgttaacggagatgacttaagttggcagcagtaatcttcttttaggagcttgtac
cacagtcttgcacataagtgcagatttggctcaagtaaagagaatttcctcaaca
ctaacttcactgggataatcagcagcgtaactaccctaaaagcatatcactagcc
aaagagggaaatatctgttcttcttactgtgcctatattaagactagtacaaatg
tggtgtgtcttccaactttcattgaaaatgccatatctataccatattttattcg
agtcactgatgatgtaatgatatattttttcattattatagtagaatatttttat
ggcaagatatttgtggtcttgatcatacctattaaaataatgccaaacaccaaat
atgaattttatgatgtacactttgtgcttggcattaaaagaaaaaaacacacatc
ctggaagtctgtaagttgttttttgttactgtaggtcttcaaagttaagagtgta
agtgaaaaatctggaggagaggataatttccactgtgtggaatgtgaatagttaa
atgaaaagttatggttatttaatgtaattattacttcaaatcctttggtcactgt
gatttcaagcatgttttctttttctcctttatatgactttctctgagttgggcaa
agaagaagctgacacaccgtatgttgttagagtcttttatctggtcaggggaaac
aaaatcttgacccagctgaacatgtcttcctgagtcagtgcctgaatctttattt
tttaaattgaatgttccttaaaggttaacatttctaaagcaatattaagaaagac
tttaaatgttattttggaagacttacgatgcatgtatacaaacgaatagcagata
atgatgactagttcacacataaagtccttttaaggagaaaatctaaaatgaaaag
tggataaacagaacatttataagtgatcagttaatgcctaagagtgaaagtagtt
ctattgacattcctcaagatatttaatatcaactgcattatgtattatgtctgct
taaatcatttaaaaacggcaaagaattatatagactatgaggtaccttgctgtgt
aggaggatgaaaggggagttgatagtctcataaaactaatttggcttcaagtttc
atgaatctgtaactagaatttaattttcaccccaataatgttctatatagccttt
gctaaagagcaactaataaattaaacctattctttctgtg
(Human Gremlin-1; coding sequence)
SEQ ID NO: 37
Atgagccgcacagcctacacggtgggagccctgcttctcctcttggggaccctgc
tgccggctgctgaagggaaaaagaaagggtcccaaggtgccatccccccgccaga
caaggcccagcacaatgactcagagcagactcagtcgccccagcagcctggctcc
aggaaccgggggcggggccaagggcggggcactgccatgcccggggaggaggtgc
tggagtccagccaagaggccctgcatgtgacggagcgcaaatacctgaagcgaga
ctggtgcaaaacccagccgcttaagcagaccatccacgaggaaggctgcaacagt
cgcaccatcatcaaccgcttctgttacggccagtgcaactctttctacatcccca
ggcacatccggaaggaggaaggttcctttcagtcctgctccttctgcaagcccaa
gaaattcactaccatgatggtcacactcaactgccctgaactacagccacctacc
aagaagaagagagtcacacgtgtgaagcagtgtcgttgcatatccatcgatttgg
attaa

### REFERENCES

Hingorani, S. R., L. Wang, A. S. Multani, C. Combs, T. B. Deramaudt, R. H. Hruban, et al. Trp53R172H and KrasGl2D cooperate to promote chromosomal instability and widely metastatic pancreatic ductal adenocarcinoma in mice. Cancer Cell 2005; 7:469-83.
Kobayashi, Y., S. O. Lim and H. Yamaguchi. Oncogenic signaling pathways associated with immune evasion and resistance to immune checkpoint inhibitors in cancer. Semin Cancer Biol; 2020; 65:51-64.
Kozar, I., C. Margue, S. Rothengatter, et al. Many ways to resistance: How melanoma cells evade targeted therapies. Biochim Biophys Acta Rev Cancer; 2019; 1871(2):313-322.
Martinelli, E., F. Morgillo, T. Troiani, et al. Cancer resistance to therapies against the EGFR-RAS-RAF pathway: The role of MEK. Cancer Treat Rev; 2017; 53:61-69.
Yu, Y., L. Cheng, B. Yan, et al. Overexpression of Gremlin 1 by sonic hedgehog signaling promotes pancreatic cancer progression. Int J Oncol; 2018; 53(6):2445-2457).

1. An anti-GREM1 antagonist for use in a method for the treatment or prevention of a cancer, wherein the method further comprises administering an inhibitor of Ras-Raf-MEK-ERK signalling.

2. The anti-GREM1 antagonist for use according to claim 1, wherein:
(a) the cancer is a solid cancer.
(b) the cancer has stromal GREM1 overexpression;
(c) the cancer is a cancer associated with Ras-Raf-MEK-ERK pathway dysregulation;
(d) the cancer displays Ras-Raf-MEK-ERK pathway dysregulation following exposure to an anti-GREM1 antagonist;
(e) the cancer contains a mutation in a Ras gene optionally KRAS, NRAS and/or HRAS;
(f) the cancer contains a mutation in a Raf gene; optionally ARAF, BRAF and/or CRAF;
(g) the cancer is a cancer that is poorly responsive, non-responsive or refractory to treatment with an inhibitor of Ras-Raf-MEK-ERK signalling; and/or
(h) the cancer is selected from colorectal cancer, multiple myeloma, pancreatic cancer, bladder cancer, breast cancer, lung cancer, stomach cancer, duodenal cancer, oesophageal cancer, head and neck cancer, prostate cancer, glioma, endometrial cancer, liver cancer, spleen cancer, bone-resident cancer, melanoma and osteosarcoma.

3. The anti-GREM1 antagonist for use according to claim 2 (h), wherein:
(a) the cancer is pancreatic cancer; optionally wherein:
(i) the pancreatic cancer is an exocrine pancreatic cancer; and/or
(ii) the pancreatic cancer is pancreatic ductal adenocarcinoma (PDAC);
(b) the cancer is multiple myeloma or colorectal cancer; optionally KRAS/BRAF mutated colorectal cancer;
(c) the cancer is lung cancer; optionally wherein the cancer is a non-small cell lung cancer (NSCLC); or
(d) the cancer is a melanoma; optionally a BRAF-mutated melanoma.

4. The anti-GREM1 antagonist for use according to any one of the preceding claims, wherein:
(a) the cancer has epithelial GREM1 overexpression; optionally wherein the cancer is a GREM1-initiated cancer;
(b) the cancer is a disseminated cancer; and/or
(c) the cancer is an established cancer.

5. The anti-GREM1 antagonist for use according to any one of the preceding claims, wherein the antagonist is a peptide, a protein, an antibody, a polynucleotide, an oligonucleotide, an antisense RNA, a small interfering RNA (siRNA), a small molecule inhibitor or a small hairpin RNA (shRNA).

6. The anti-GREM1 antagonist for use according to claim 5, wherein:
(a) the antagonist is an antibody which binds to an epitope on Gremlin-1 comprising at least one residue selected from Ile131, Lys147, Lys148, Phe149, Thr150, Thr151, Arg169, Lys174 and Gln175, wherein the residue numbering is according to SEQ ID NO: 1; optionally wherein:
(i) the antibody binds an epitope comprising all of Ile131, Lys147, Lys148, Phe149, Thr150, Thr151, Arg169, Lys174 and Gln175; and/or
(ii) Lys147, Lys148, Phe149, Thr150, Thr151, Arg169, Lys174 and Gln175 are located on the same Gremlin-1 monomer and Ile131 is located on the second Gremlin-1 monomer;
(b) the antagonist is an anti-Gremlin-1 antibody which comprises heavy chain complementarity determining region (HCDR) sequences contained within a heavy chain variable region (HCVR) of SEQ ID NO: 10 or 12 and/or light chain complementarity determining region (LCDR) sequences contained within a light chain variable region (LCVR) of SEQ ID NO: 11 or 13; or
(c) the antagonist is an anti-Gremlin-1 antibody which comprises at least one HCDR sequence selected from SEQ ID NOs: 3, 4, 5 and 6 and/or at least one LCDR sequence selected from SEQ ID NOs: 7, 8 and 9; optionally wherein:
(i) the anti-Gremlin-1 antibody comprises a HCDR3 sequence of SEQ ID NO: 6;
(ii) the anti-Gremlin-1 antibody comprises an HCDR1/HCDR2/HCDR3 sequence combination selected from SEQ ID NOs: 4/5/6 or from SEQ ID NOs:3/5/6 and/or an LCDR1/LCDR2/LCDR3 sequence combination selected from SEQ ID NOs: 7/8/9;
(iii) the anti-Gremlin-1 antibody comprises a HCDR1/HCDR2/HCDR3/LCDR1/LCDR2/LCDR3 sequence combination of SEQ ID NOs: 4/5/6/7/8/9 or SEQ ID NOs: 3/5/6/7/8/9; and/or
(iv) the anti-Gremlin-1 antibody comprises a heavy chain variable region (HCVR) sequence of SEQ ID NO: 10 or 12 and/or a light chain variable region (LCVR) sequence of SEQ ID NO: 11 or 13, or sequences which are at least 95% identical thereto.

7. The anti-GREM1 antagonist for use according to claim 6(c)(iv), wherein:
(a) the anti-Gremlin-1 antibody comprises a HCVR and LCVR sequence pair of SEQ ID NOs: 10/11 or 12/13 or sequences which are at least 95% identical thereto; optionally wherein the anti-Gremlin-1 antibody comprises HCDR1/HCDR2/HCDR3/LCDR1/LCDR2/LCDR3 sequences consisting of SEQ ID NOs: 4/5/6/7/8/9 or SEQ ID NOs: 3/5/6/7/8/9 and the remainder of the HCVR and LCVR comprise at least 95% identity to SEQ ID NOs: 10, 11, 12 and/or 13 respectively; and/or
(b) the anti-Gremlin-1 antibody comprises a heavy chain of SEQ ID NO: 14, 16, 18, 22, 28, 30, 32 or 34 and/or a light chain of SEQ ID NO: 15, 17, 19, 23, 29, 31, 33 or 35, or sequences which are at least 95% identical thereto.

8. The anti-GREM1 antagonist for use according to claim 7(b), wherein the anti-Gremlin-1 antibody comprises a heavy and light chain pair of SEQ ID NOs: 14/15, 16/17, 18/19, 22/23, 28/29 or 30/31, 32/33, 34/35, or sequences which are at least 95% identical thereto; optionally wherein the HCDR1/HCDR2/HCDR3/LCDR1/LCDR2/LCDR3 sequences of the antibody consist of SEQ ID NOs: 4/5/6/7/8/9 or SEQ ID NOs: 3/5/6/7/8/9 and the remainder of the heavy and light chains comprise at least 95% identity to SEQ ID NOs: 14, 15, 16 and/or 17 respectively.

9. The anti-GREM1 antagonist for use according to claim 5, wherein:
(a) the antagonist is an antibody which competes for binding to Gremlin-1 with an antibody as defined in any one of claims 6(a)(i)-7; or
(b) the antagonist is an antibody which binds the same epitope on Gremlin-1 as an antibody defined in any one of claims 6(a)(i)-7.

10. The anti-GREM1 antagonist for use according to any one of claims 5-9, wherein:
(a) the antagonist antibody is a chimeric, human or humanised antibody; and/or
(b) the antagonist antibody is a Fab, modified Fab, Fab′, modified Fab′, F(ab′)₂, Fv, single domain antibody or an scFv.

11. The anti-GREM1 antagonist for use according to claim 5, wherein the antagonist is a polynucleotide encoding an antibody as defined in any one of claims 6-10, or an expression vector carrying said polynucleotide.

12. The anti-GREM1 antagonist for use according to any one of claims 5-10, wherein the antagonist antibody is comprised in pharmaceutical composition further comprising a pharmaceutically acceptable adjuvant and/or carrier.

13. The anti-GREM1 antagonist for use according to any one of the preceding claims wherein:
(a) the inhibitor of Ras-Raf-MEK-ERK signalling downregulates Mapk1, Nras, Kras and/or Myc signalling; and/or
(b) the inhibitor of Ras-Raf-MEK-ERK signalling is a MEK inhibitor; optionally an allosteric MEK inhibitor.

14. The anti-GREM1 antagonist for use according to claim 13(b) wherein:
(a) the MEK inhibitor is a MEK1 and/or MEK2 inhibitor; and/or
(b) the MEK inhibitor is selumetinib (AZD6244), trametinib (GSK-1120212), cobimetinib (GDC-0973), binimetinib (MEK162), CI-1040 (PD184352), Mirdametinib (PD0325901), TAK733, refametinib (RDEA119/Bay 86-9766), RO-5126766, RO5068760, Pimasertib (AS703026), AZD8330, GDC-0623, RO-4987655, WX-554 (UCB-554), HL-085, ARRY-300, ClnQ-03, G-573, PD184161, PD318088, PD98059, U0126 or SL327; optionally wherein:
(i) the MEK inhibitor is selumetinib (AZD6244);
(ii) the MEK inhibitor is trametinib (GSK-1120212);
(iii) the MEK inhibitor is WX-554 (UCB-554);
(iv) the MEK inhibitor is selumetinib (AZD6244) and the cancer is pancreatic cancer; optionally PDAC; or
(v) the MEK inhibitor is trametinib (GSK-1120212) and the cancer is pancreatic cancer; optionally PDAC;
(vi) the MEK inhibitor is WX-554 (UCB-554) and the cancer is pancreatic cancer; optionally PDAC.

15. The anti-GREM1 antagonist for use according to claim 13(b), wherein the MEK inhibitor is selected from the group consisting of:
(a) N-(2-fluoro-4-iodophenyl)-3-(morpholin-4-ylcarbonyl)thieno[2,3-b]pyridin-2-amine;
(b) N-(2-fluoro-4-iodophenyl)-3-[(4-methylpiperazin-1-yl)carbonyl]thieno[2,3-b]pyridin-2-amine;
(c) [2-[((2-fluoro-4-iodophenyl)amino]thieno[2,3-b]pyridin-3-yl](pyrrolidin-1-yl)methanone;
(d) (1-{2-[(2-fluoro-4-iodophenyl)amino]thieno[2,3-b]pyridine-3-carbonyl}piperidin-4-yl)-carbamic acid tert-butyl ester;
(e) 2-[((2-fluoro-4-iodophenyl)amino]thieno[2,3-b]pyridine-3-carboxylic acid (4-aminopiperidin-1-yl)amide dihydro-chloride;
(f) (1-{2-[(2-fluoro-4-iodophenyl)amino]thieno[2,3-b]pyridine-3-carbonyl}azetidin-3-ylmethyl)carbamic acid tert-butyl ester;
(g) [3-((aminomethyl)azetidin-1-yl]-{2-[(2-fluoro-4-iodophenyl)amino]thieno[2,3-b]pyridin-3-yl}-methanone;
(h) (1-{2-[(2-fluoro-4-iodophenyl)amino]thieno[2,3-b]pyridine-3-carbonyl}-(3R)-pyrrolidin-3-yl)carbamic acid tert-butyl ester;
(i) (1-{2-[(2-fluoro-4-iodophenyl)amino]thieno[2,3-b]pyridine-3-carbonyl}-(3S)-pyrrolidin-3-yl)carbamic acid tert-butyl ester;
(j) (1-{2-[(2-fluoro-4-iodophenyl)amino]thieno[2,3-b]pyridine-3-carbonyl}azetidin-3-yl)-carbamic acid tert-butyl ester;
(k) [(3R)-3-aminopyrrolidin-1-yl]-{2-[(2-fluoro-4-iodophenyl)amino]thieno[2,3-b]pyridin-3-yl}-methanone;
(l) [(3S)-3-aminopyrrolidin-1-yl]-{2-[(2-fluoro4-iodophenyl)amino]thieno[2,3-b]pyridin-3-yl}-methanone;
(m) (3-aminoazetidin-1-yl)-{2-[(2-fluoro-4-iodophenyl)amino]thieno[2,3-b]pyridin-3-yl]-methanone;
(n) {2-[((2-fluoro-4-iodophenyl)amino]thieno[2,3-b]pyridin-3-yl}-(3-hydroxyazetidin-1-yl)-methanone;
(o) {2-[((2-fluoro-4-iodophenyl)amino]thieno[2,3-b]pyridin-3-yl}-[(3R)-3-hydroxypyrrolidin-1-yl]-methanone;
(p) {2-[((2-fluoro-4-iodophenyl)amino]thieno[2,3-b]pyridin-3-yl}-[(3S)-3-hydroxypyrrolidin-1-yl]-methanone;
(q) {2-[((2-fluoro-4-iodophenyl)amino]thieno[2,3-b]pyridin-3-yl}-[2-(hydroxymethyl)-piperidin-1-yl]-methanone;
(r) {2-[((2-fluoro-4-iodophenyl)amino]thieno[2,3-b]pyridin-3-yl}-[(3S)-3-(hydroxymethyl)-morpholin-4-yl]-methanone;
(s) 4-{2-[(2-fluoro-4-iodophenyl)amino]thieno[2,3-b]pyridine-3-carbonyl}-(3R)-3-(hydroxymethyl)piperazine-1-carboxylic acid tert-butyl ester;
(t) 2-[4({2-[(2-fluoro-4-iodophenyl)amino]thieno[2,3-b]pyridin-3-yl}carbonyl)piperazin-1-yl]ethanol;
(u) 3-((1,4-diazepan-1-ylcarbonyl)-N-(2-fluoro-4-iodophenyl)thieno[2,3-b]pyridin-2-amine;
(v) N-(2-fluoro-4-iodophenyl)-3-(piperazin-1-ylcarbonyl)thieno[2,3-b]pyridin-2-amine;
(w) ethyl [4-({2-[(2-fluoro-4-iodophenyl)amino]thieno[2,3-b]pyridin-3-yl}carbonyl)-piperazin-1-yl]acetate;
(x) [4-({2-[(2-fluoro-4-iodophenyl)amino]thieno[2,3-b]pyridin-3-yl}carbonyl)piperazin-1-yl]acetic acid;
(y) {2-[((2-fluoro-4-iodophenyl)amino]thieno[2,3-b]pyridin-3-yl}-[(2R)-2-(methoxymethyl)-pyrrolidin-1-yl]-methone; and
(z) {2-[((2-fluoro-4-iodophenyl)amino]thieno[2,3-b]pyridin-3-yl}-[(2R)-2-(hydroxymethyl)-piperazin-1-yl]-methanone.

16. The anti-GREM1 antagonist for use according to claim 13(b), wherein the MEK inhibitor is represented by formula (II), and pharmaceutically acceptable salts, solvates and N-oxides thereof: 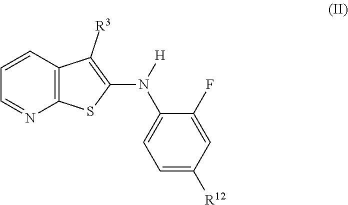
wherein
R¹² represents halogen;
R³ represents CONR^{b}R^{c}; and
R^{b} and R^{c}, when taken together with the nitrogen atom to which they are both attached, represent azetidin-1-yl, pyrrolidin-1-yl, piperidin-1-yl, morpholin-4-yl, thiomorpholin-4-yl, piperazin-1-yl, homopiperidin-l-yl, homo-morpholin-4-yl or homopiperazin-1-yl, any of which groups may be optionally substituted by one or more substituents selected from C₁₋₆ alkyl, C₁₋₆ alkoxy, hydroxy, hydroxy(C₁₋₆)akyl, amino-(C₁₋₆)alkyl, (amino)(hydroxy)(C₁₋₆)alkyl, halogen, oxo, C₂-6 akylcarbonyl, carboxy, C₂₋₆ alkoxycarbonyl, di(C₁₋₆)alhylhydrazinylcarbonyl, amino, C₁₋₆ alkylamino, di(C₁₋₆)alkyl-amino, C₂₋₆ alkylcarbonylamino, aminocarbonylamino, aminocarbonyl, C₁₋₆ alkylaminocarbonyl, di(C₁₋₆)alkylaminocarbonyl, aminosulfonyl, C₁₋₆ alkylsulfonyl, C₁₋₆ alkylaminocarbonyl (C₁₋₆)alkyl, C₁₋₆ alkoxy(C₁₋₆)alkyl, carboxy(C₁₋₆)alkyl, C₂₋₆ alkoxycarbonyl(C₁₋₆)alkyl, C₂₋₆ alkoxycarbonylamino and C₂₋₆ alkoxycarbonylamino-(C₁₋₆)alkyl;
optionally wherein R¹² represents bromo or iodo.

17. The anti-GREM1 antagonist for use according to any one of claims 1-13(a), wherein the inhibitor of Ras-Raf-MEK-ERK signalling is an ERK inhibitor; optionally wherein:
(a) the ERK inhibitor is ulixertinib, temuterkib, JSI-1187, ravoxertinib ASN007 or MK-8353; and/or
(b) the ERK inhibitor is ulixertinib; further optionally wherein the ERK inhibitor is ulixertinib and the cancer is pancreatic cancer; optionally PDAC.

18. The anti-GREM1 antagonist for use according to any one of the preceding claims, wherein the method further comprises administering an additional anti-cancer agent.

19. An inhibitor of Ras-Raf-MEK-ERK signalling for use in a method for the treatment or prevention of cancer wherein the method comprises further administering an anti-GREM1 antagonist.

20. The inhibitor of Ras-Raf-MEK-ERK signalling for use according to claim 19, wherein said cancer, said antagonist and/or said method are as defined in any one of claims 1-18.

21. The inhibitor of Ras-Raf-MEK-ERK signalling for use according to claim 19 or claim 20, wherein:
(a) the inhibitor of Ras-Raf-MEK-ERK signalling is a MEK inhibitor; optionally wherein:
(i) the inhibitor is selumetinib (AZD6244);
(ii) the inhibitor is trametinib (GSK-1120212); or
(iii) the inhibitor is WX-554 (UCB-554); or
(b) the inhibitor of Ras-Raf-MEK-ERK signalling is an ERK inhibitor; optionally wherein said ERK inhibitor is ulixertinib.

22. A method of treating a cancer comprising administering a therapeutically effective amount of an anti-GREM1 antagonist in combination with a therapeutically effective amount of an inhibitor of Ras-Raf-MEK-ERK signalling to a subject in need thereof.

23. The method according to claim 22, wherein said cancer, said antagonist and/or said method are as defined in any one of claims 1-21.

24. A composition or kit comprising an anti-GREM1 antagonist and an inhibitor of Ras-Raf-MEK-ERK signalling.

25. The composition or kit according to claim 24, wherein:
(a) the anti-GREM1 antagonist is as defined in any one of claims 5 to 11; and/or
(b) the the inhibitor of Ras-Raf-MEK-ERK signalling is as defined in any one of claims 13(a) to 17.

26. A method for determining:
(a) whether or not a patient having or suspected of having or being at risk of developing cancer is likely to respond to a combination treatment with a GREM1 antagonist and an inhibitor of Ras-Raf-MEK-ERK signalling, which method comprises measuring stromal expression of GREM1, epithelial expression of GREM1 and/or Ras-Raf-MEK-ERK pathway signalling in the patient, and thereby predicting whether or not the patient is likely to respond to treatment with the combination; or
(b) whether or not a patient having or suspected of having or being at risk of developing cancer is likely to respond to a combination treatment with a GREM1 antagonist and an inhibitor of Ras-Raf-MEK-ERK signalling, which method comprises measuring stromal expression of GREM1 and/or epithelial expression of GREM1 in the patient and determining whether the patient has a mutation in a RAS gene or a RAF gene, and thereby predicting whether or not the patient is likely to respond to treatment with the combination; or
(c) whether or not a patient having or suspected of having or being at risk of developing cancer is likely to respond to a combination treatment with a GREM1 antagonist and an inhibitor of Ras-Raf-MEK-ERK signalling, which method comprises measuring an Ras-Raf-MEK-ERK signalling pathway signalling in the patient in response to treatment with an anti-GREM1 antagonist, and thereby predicting whether or not the patient is likely to respond to treatment with the combination.


SEQUENCE LISTING (37 sequences)

SEQ ID NO: 1 - protein, 184 aa, Homo sapiens
MSRTAYTVGALLLLLGTLLPAAEGKKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 2 - protein, 139 aa, synthetic construct
MGSSHHHHHHSSGENLYFQGSAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 3 - protein, 10 aa, synthetic construct
GYTFTDYYMH

SEQ ID NO: 4 - protein, 5 aa, synthetic construct
DYYMH

SEQ ID NO: 5 - protein, 17 aa, synthetic construct
LVDPEDGETIYAEKFQG

SEQ ID NO: 6 - protein, 15 aa, synthetic construct
DARGSGSYYPNHFDY

SEQ ID NO: 7 - protein, 17 aa, synthetic construct
KSSQSVLYSSNNKNYLA

SEQ ID NO: 8 - protein, 7 aa, synthetic construct
WASTRES

SEQ ID NO: 9 - protein, 8 aa, synthetic construct
QQYYDTPT

SEQ ID NO: 10 - protein, 124 aa, synthetic construct
QVQLVESGAEVKKPGATVKISCKVSGYTFTDYYMHWVQQAPGKGLEWMGLVDPEDGETIYAEKFQGRVTITADTSTDTAYMELSSLRSEDTAVYYCATDARGSGSYYPNHFDYWGQGTLVTVSS

SEQ ID NO: 11 - protein, 112 aa, synthetic construct
DIVMTQSPDSLAVSLGERATINCKSSQSVLYSSNNKNYLAWYQQKPGQPPKLLIYWASTRESGVPDRFSGSGSGTDFTLTINSLQAEDVAVYFCQQYYDTPTFGQGTRLEIK

SEQ ID NO: 12 - protein, 124 aa, synthetic construct
QVQLVQSGAEVKKPGATVKISCKVSGYTFTDYYMHWVQQAPGKGLEWMGLVDPEDGETIYAEKFQGRVTITADTSTDTAYMELSSLRSEDTAVYYCATDARGSGSYYPNHFDYWGQGTLVTVSS

SEQ ID NO: 13 - protein, 112 aa, synthetic construct
DIVMTQTPDSLAVSLGERATINCKSSQSVLYSSNNKNYLAWYQQKPGQPPKLLIYWASTRESGVPDRFSGSGSGTDFTLTINSLQAEDVAVYFCQQYYDTPTFGQGTRLEIK

SEQ ID NO: 14 - protein, 448 aa, synthetic construct
QVQLVESGAEVKKPGATVKISCKVSGYTFTDYYMHWVQQAPGKGLEWMGLVDPEDGETIYAEKFQGRVTITADTSTDTAYMELSSLRSEDTAVYYCATDARGSGSYYPNHFDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 15 - protein, 219 aa, synthetic construct
DIVMTQSPDSLAVSLGERATINCKSSQSVLYSSNNKNYLAWYQQKPGQPPKLLIYWASTRESGVPDRFSGSGSGTDFTLTINSLQAEDVAVYFCQQYYDTPTFGQGTRLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 16 - protein, 454 aa, synthetic construct
QVQLVQSGAEVKKPGATVKISCKVSGYTFTDYYMHWVQQAPGKGLEWMGLVDPEDGETIYAEKFQGRVTITADTSTDTAYMELSSLRSEDTAVYYCATDARGSGSYYPNHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 219 aa, synthetic construct
DIVMTQTPDSLAVSLGERATINCKSSQSVLYSSNNKNYLAWYQQKPGQPPKLLIYWASTRESGVPDRFSGSGSGTDFTLTINSLQAEDVAVYFCQQYYDTPTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 227 aa, synthetic construct
QVQLVESGAEVKKPGATVKISCKVSGYTFTDYYMHWVQQAPGKGLEWMGLVDPEDGETIYAEKFQGRVTITADTSTDTAYMELSSLRSEDTAVYYCATDARGSGSYYPNHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 19 - protein, 219 aa, synthetic construct
DIVMTQSPDSLAVSLGERATINCKSSQSVLYSSNNKNYLAWYQQKPGQPPKLLIYWASTRESGVPDRFSGSGSGTDFTLTINSLQAEDVAVYFCQQYYDTPTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 118 aa, synthetic construct
AMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 21 - protein, 160 aa, synthetic construct
KKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 22 - protein, 451 aa, synthetic construct
QVQLVESGAEVKKPGATVKISCKVSGYTFTDYYMHWVQQAPGKGLEWMGLVDPEDGETIYAEKFQGRVTITADTSTDTAYMELSSLRSEDTAVYYCATDARGSGSYYPNHFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 219 aa, synthetic construct; identical to SEQ ID NO: 19

SEQ ID NO: 24 - DNA, 1362 bp, synthetic construct
  source 1..1362: mol_type = other DNA
caagtgcaactggtggaatccggggccgaagtgaaaaagcccggagccactgtgaagatctcttgcaaagtgtccggctacaccttcaccgactattacatgcactgggtccagcaggcacctgggaagggccttgagtggatgggtctggtcgatcccgaggacggcgaaactatctacgccgagaagttccagggtcgcgtcaccatcaccgccgacacttccaccgacaccgcgtacatggagctgtccagcttgaggtccgaggacacagccgtgtactactgcgccacggatgctcggggaagcggcagctactacccgaaccacttcgactactggggacagggcactctcgtgactgtctcgagcgcttctacaaagggcccctccgtgttcccgctcgctccatcatcgaagtctaccagcggaggcactgcggctctcggttgcctcgtgaaggactacttcccggagccggtgaccgtgtcgtggaacagcggagccctgaccagcggggtgcacacctttccggccgtcttgcagtcaagcggcctttactccctgtcatcagtggtgactgtcccgtccagctcattgggaacccaaacctacatctgcaatgtgaatcacaaacctagcaacaccaaggttgacaagaaagtcgagcccaaatcgtgtgacaagactcacacttgtccgccgtgcccggcacccgaactgctgggaggtcccagcgtctttctgttccctccaaagccgaaagacacgctgatgatctcccgcaccccggaggtcacttgcgtggtcgtggacgtgtcacatgaggacccagaggtgaagttcaattggtacgtggatggcgtcgaagtccacaatgccaaaactaagcccagagaagaacagtacaattcgacctaccgcgtcgtgtccgtgctcacggtgttgcatcaggattggctgaacgggaaggaatacaagtgcaaagtgtccaacaaggcgctgccggcaccgatcgagaaaactatctccaaagcgaagggacagcctagggaacctcaagtctacacgctgccaccatcacgggatgaactgactaagaatcaagtctcactgacttgtctggtgaaggggttttaccctagcgacattgccgtggagtgggaatccaacggccagccagagaacaactacaagactacccctccagtgctcgactcggatggatcgttcttcctttactcgaagctcaccgtggataagtcccggtggcagcagggaaacgtgttctcctgctcggtgatgcatgaagccctccataaccactatacccaaaagtcgctgtccctgtcgccgggaaag

SEQ ID NO: 25 - DNA, 657 bp, synthetic construct
  source 1..657: mol_type = other DNA
gacattgtgatgacccagtcccccgattcgcttgcggtgtccctgggagaacgggccaccattaactgcaagagctcacagtccgtcctgtattcatcgaacaacaagaattacctcgcatggtatcagcagaagcctggacagcctcccaagctgctcatctactgggctagcacccgcgaatccggggtgccggatagattctccggatcgggttcgggcactgacttcactctgactatcaactcactgcaagccgaggatgtcgcggtgtacttctgtcagcagtactacgacaccccgacctttggacaaggcaccagactggagattaagcgtacggtggccgctccctccgtgttcatcttcccaccctccgacgagcagctgaagtccggcaccgcctccgtcgtgtgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgc

SEQ ID NO: 26 - DNA, 1353 bp, synthetic construct
  source 1..1353: mol_type = other DNA
caagtgcaactggtggaatccggggccgaagtgaaaaagcccggagccactgtgaagatctcttgcaaagtgtccggctacaccttcaccgactattacatgcactgggtccagcaggcacctgggaagggccttgagtggatgggtctggtcgatcccgaggacggcgaaactatctacgccgagaagttccagggtcgcgtcaccatcaccgccgacacttccaccgacaccgcgtacatggagctgtccagcttgaggtccgaggacacagccgtgtactactgcgccacggatgctcggggaagcggcagctactacccgaaccacttcgactactggggacagggcactctcgtgactgtctcgagcgcttctacaaagggcccctccgtgttccctctggccccttgctcccggtccacctccgagtctaccgccgctctgggctgcctggtcaaggactacttccccgagcccgtgacagtgtcctggaactctggcgccctgacctccggcgtgcacaccttccctgccgtgctgcagtcctccggcctgtactccctgtcctccgtcgtgaccgtgccctcctccagcctgggcaccaagacctacacctgtaacgtggaccacaagccctccaacaccaaggtggacaagcgggtggaatctaagtacggccctccctgccccccctgccctgcccctgaatttctgggcggaccttccgtgttcctgttccccccaaagcccaaggacaccctgatgatctcccggacccccgaagtgacctgcgtggtggtggacgtgtcccaggaagatcccgaggtccagttcaattggtacgtggacggcgtggaagtgcacaatgccaagaccaagcccagagaggaacagttcaactccacctaccgggtggtgtccgtgctgaccgtgctgcaccaggactggctgaacggcaaagagtacaagtgcaaggtgtccaacaagggcctgccctccagcatcgaaaagaccatctccaaggccaagggccagccccgcgagccccaggtgtacaccctgccccctagccaggaagagatgaccaagaaccaggtgtccctgacctgtctggtcaagggcttctacccctccgacattgccgtggaatgggagtccaacggccagcccgagaacaactacaagaccaccccccctgtgctggacagcgacggctccttcttcctgtactctcggctgaccgtggacaagtcccggtggcaggaaggcaacgtcttctcctgctccgtgatgcacgaggccctgcacaaccactacacccagaagtccctgtccctgagcctgggcaag

SEQ ID NO: 27 - DNA, 657 bp, synthetic construct; identical to SEQ ID NO: 25
  source 1..657: mol_type = other DNA

SEQ ID NO: 28 - protein, 448 aa, synthetic construct
QVQLVQSGAEVKKPGATVKISCKVSGYTFTDYYMHWVQQAPGKGLEWMGLVDPEDGETIYAEKFQGRVTITADTSTDTAYMELSSLRSEDTAVYYCATDARGSGSYYPNHFDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 29 - protein, 219 aa, synthetic construct
DIVMTQTPDSLAVSLGERATINCKSSQSVLYSSNNKNYLAWYQQKPGQPPKLLIYWASTRESGVPDRFSGSGSGTDFTLTINSLQAEDVAVYFCQQYYDTPTFGQGTRLEIKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 30 - protein, 454 aa, synthetic construct
QVQLVESGAEVKKPGATVKISCKVSGYTFTDYYMHWVQQAPGKGLEWMGLVDPEDGETIYAEKFQGRVTITADTSTDTAYMELSSLRSEDTAVYYCATDARGSGSYYPNHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 219 aa, synthetic construct; identical to SEQ ID NO: 19

SEQ ID NO: 32 - protein, 227 aa, synthetic construct
QVQLVQSGAEVKKPGATVKISCKVSGYTFTDYYMHWVQQAPGKGLEWMGLVDPEDGETIYAEKFQGRVTITADTSTDTAYMELSSLRSEDTAVYYCATDARGSGSYYPNHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 33 - protein, 219 aa, synthetic construct; identical to SEQ ID NO: 17

SEQ ID NO: 34 - protein, 451 aa, synthetic construct
QVQLVQSGAEVKKPGATVKISCKVSGYTFTDYYMHWVQQAPGKGLEWMGLVDPEDGETIYAEKFQGRVTITADTSTDTAYMELSSLRSEDTAVYYCATDARGSGSYYPNHFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 219 aa, synthetic construct; identical to SEQ ID NO: 17

SEQ ID NO: 36 - DNA, 16654 bp, Homo sapiens
  source 1..16654: mol_type = genomic DNA
actcggtgcgccttccgcggaccgggcgacccagtgcacggccgccgcgtcactctcggtcccgctgaccccgcgccgagccccggcggctctggccgcggccgcactcagcgccacgcgtcgaaagcgcaggccccgaggacccgccgcactgacaggtgagcgcggacgcacccggcagggatgtgagtgggcggagggaagagggccgcaaaccaacccaggacccgctcagttccacgcgcggcagccctccgtgcgcgcaggctcgggtgcgttgttcgcgggggtgaattgtgaagaaccatcgcggggtccttcctgctgaggccgcggacaccgtgacctcgctgctctgggtctgcagggaaacgtaggaaaaaaagttgtcaggagcgggcaggatgacccccacatcccgtttccacctcccggaggcccccgaacacgctcctggtgctggtggcagcagcgcctggcagacgcgcccgcttagcgagggcgcgaagtccaggccgccagagcgcaggagcatccggacctgctagtcggccgctgactgcgcggcgagttgccttgagagggtcccatgtgcttggggcgccgcgctgggtctgggggcgtcttggggcgcccattggagtccgcgggttggagcatccggagaatccatgatgtgtgcatttgccgatccccgaggtgagatggagactggcaagggcagagccgctgtgttcagccacagcggaaaaccgaacggtgggtaatccgacagctgcggtgcggggcgcggccctggccgcggggtccagcgaacccgcagtgctcacaaggcagacaccacacgcgctcgcggaccggccacgcactcgcgggcgctcgcttctctactccagcctcttccccgccccgcgcacgcccgagctgaatggtagacgttctggcgccgggcagcggccaccggctggttcccacttccgcgcgcaccccttaaactgtgttctagaggccccagcctcgccttgcagcgcctcactagctcctgaggactagggactggcggctgaggcgggttggcggctgcaacgagctgggcgtctttcgttctctctcgctgcctggctggctccgctggcccctccacagcttgcggagcaaggccatagcaggggagtgggaggtatattggggctgtcacctccttgctggccggagttatttgtagactacagactccggaagaacagacgcgccaccgctctcgcttggcattgccttcggatcgcagctcctccttgggggtgccccagcttggcgtttatttgcctgcgccaggctctggcgacggtcaccgggccaggcggggagggacggacggcaggtgaccagcctctgctgtgaagaaattcctgcgcgcccggagctgtccctaatgcattcccgggtcgaatccgtctactgccttcccctcctcgaccgactccgaatctcggctcttatagacagaaatacagcctcagcgttaggggttaaaatccccctcttaaacggtccgagggcagagaggtgaccaccgataggtaattggatctcctgctggaaagagcaaatctgagcggtgtgcgcgtctgtttatgttccccttcgagatggtgccaggacacgaactgattaaaacaatctattgtgttaagtgggtcactagggttttaagctgtcccagggaccccagagtagtggcttccttctggctgtacacacaagttaaataaatagcgtagaagaggttaagataaccccattctagggtgaggagtcctctttcatccctagggcttccccctccccttttctctttttttggaaggagggggagcatgagagtcttgagggggggatgtacttttcaaagcaaggagggaaagatcttaagaaaactatatattctcactgccccccaagccaagtctataacagtaggtgatttgattactatctctggataaatggcactgtcaaattgttaatattaactatttcagggatttttagcagggtagtggcagtatgtgtgcgtgtgtgtgtgtgtgtctgtgtgtgtgtgtttaacctccaggtcattgtaggaattagagtcttttgtaaactttgtaatttcacaggtttcctattttcttaaaagttcatttttagtgaaatgttttggtaacccacgctctgtaggaaatccaggttggctaatgcggtctttatgtgagtagttacacagggaaggataaaaaccttttatgtcctacatctctgaatgagggctgcctaccctgtctttgaaactaagccgaagatgccttcagtctgaatggtcaagtattaaaagtgataaaatgcaaagaaatttcatgccgcagacacctcccccaagaactgcttgttgacagcaaagctgtggaacatgttccacaacagagagtaaaggacagccaggaaatataaaccttttatgtaaaggaaaggcaggtgggggacagtggttaggggaggtgactgcagcctctaaccaaaaggcaaccatcaggcaagtgctaccagcccgtgtcttcgatctgcaaggaattttctttagttttaacatatgctcttagaaattcaaagtacaacaggaattcctgggacaagagaaatctttttattcacatgtgaacatgaagatacaaaatagataattattttatttatagcactcttcaaattgtattgcattagaaaacatatccattgacccactgttaaggacagcactgggtgtcaataggacagtggttaaggacctgtgtttggggctagatagaattgggtttaaactgctggctgggctgggcacagtggctcacaccggtaatcccagcactttgggaggccaaggaggacggatcacctgaggtcgggagttcgacaccagcctgaccaacatggaaaaatcccgtctctactaaaaacacaaaattagccaggcatggtggtgcatgcctgcaatcccagctacttgggaggctgaggcaggagaattgcttgaaaccgggaggcggaggttgtggtgagcccagatagcgccattgcattccagcctgggcaacacgagtgaaaactccgtcaaaaaaacaaaacaaaacaaacaaacaaaaaaatgctggctttcccacttatgagctgtgtgaccttggacaaatttccaacttttctgagtgtagattccctgattggtaaaaggaagatgatattatctacctcatattttgttatgaaaaataaatgataaaattgggtcagaaatcagcataatgcctggcacagtaagggcttcaaaataaaaggtagctcttattattagtaatggtgttaggaaaagtagcaatgttatacagaaccaggatatatcacagggcagttctgaaattaaatcctgaatcctggccgggtgaggtggctcacgcttgtaatcctaagcactttcggagactgaggcaggcggatcacgaggtcaggagtttgagaccagcctggccaacacactgaaatcccgtctctactaaaaatacaaaaattagctgggtgtggtggcaggcgcctataatctcagctacttgggaggctgaggcaggagaatcacttgagcccaggaggcggaggttgcagtgagctgatatcgtgccagtgcactccagcctgggtgacatctcttaaaaaaaaaaatcctgaataccacactacgcagtgactaacacatctttcactacagaacagaacctgtaacttggccgtctctcagcagtgctgctcagtgaacatttaataatttattactttctaactcgtttcttgttgacctcaagaattgtacatagtcattaactttcctaagaaaatctttgacaaacatagagctcctgagatatttcacaaccaggtggtctcctccctgtcctatgcaatgttgggccccagcctgatttagccgacctggtcttcagacttgagaggctgtttagggttcttacaacacaaaggggatgagactttatcctctacctgtgtgccaacaagggattcttcttatctccttggtgcaacttgtctgaaaaagaaagtcaacacaattatcttcttaaaagttaaagatcaaattaaaaataagctatagttttcccaaagatttagacctgagaaaaaggaatagatctttctaaaacctggcctgcacttagagcatttgtagtcacttccactatttcttatgctgagagaataatttgatgtcatgcctattgaatgtctttctaaagcttgattcatcaggaggaactgaccagaagtccatgcacagacatttggctttcactgtaattcctactcaaaactccctttcactgattatgagcaggttgcttagctaaaatgagaaatacagcaagaagagggatggaaagagctggcttaaactttcccaaagacatcatgaacactgggaacaggtcatacatataccatttttatttctcagtcctcagtattagaatattgtgttcctagaggctttgtgaaaattggaatacattgccatgacctgcacaggatagaggtggttaatatggcttaccttgcttgtagatgtttactcttgatccctaagggaaactggaaggaaagcatgctgtagagaggacctgctttgagagtatgtgtcctctgggacaagtggaatgaaagagaggatagaggcaaagagaaaaaaaggtggagatggggtgagacttgctggtggacagttgcaaagaaaactcatgatgagaataacatcacatttcttgaagaagctgaactgcattgatgggaagttgaggcagagtttgaaaagaagatatactgtctgcaaaaaagaatcaacggagatgtcaggtatcttgatgcaccctgagtctatgagatgcagcttaatttaatttaatgatatgagtaggccatttcataagtggtggaaattaccataaagtgtttgcagctaatgacggcgcatgaatgatgtcataggaacctaagtctggatgagctatggattgaatttttactatgggacacccttctacatgttgctgtggaaaagaattgtcctcaagaaatgtacatcttctgcatttcctctacatctctgatatttacaaagtgcacattatttggtgatactacaactgggatttcaagtgcacatcactactatttccttcactgaagaatcaagagcagtctgggggtggggagagcttgagtgattgacaaggatgtggcagagccctggttccactatgaattatagttcttaccctgcttactgtcactcatgtagagcagctagtgtgcctcagagcactgctgttctctgagaagctgaggtctcgattgacattcttgaagttggtgtttacttctctctgaataaacaaaggttggcaactcagacatcttacaataagtactaaagatttttgaagaataggtttttaaaaaatgaaaaaacatttcactttccatagctaataaatcttattttgaggaaaatgtacttttctttaaaaaaaaaaaaagcctgtctgtcactctagaccctttggcttagaaggtaggcacactcacatagaaacagaaagtctgtccaaattaaaactgaaaaccacagttgactaattttgaatttatagctctgctgttggcttctgcgatagtattaatttcaatggcttcaattagaaaatgaaacccatagcattccatatgagaacaggtaaaaagtcagggacatttggagttttccaagaaaaagaaagacaagtcttaggaagctctctaggatggaaggaatttgccacactgagagttagacatccaaaggatagcaattggctcttctgctcatgggcactggtgaaggcattttaaaatgcgaagaatggtacctctgtaaatcaatgaggttcataataatcatgcatttaccaaatttttataagcacctgccttgtgccaggcactgagggtaaggtgatgaataagccctcatcaactgtcagactagatatttactcaataacagatgtgaaaatgccaagaaggaaaagttgagtataaagaaagccttaagttggttcagagaaataaaattgcatttttcggatagatgtttatggaatcggccttatgaggtaaacttgtcctatgcagtgaacatacattcccagttagtctcagattggcctctgtgatgacaaactcagagggtcctggtctaggaggggtgaatttgtctggaggccattttcaggaggtatggaggaagactggggcataggcctggggccatcccatgtactcctcctctgaaatggggagcaactgaattgtgttttattttagatcttcgtccaacttgaataccagaaattcgtgaaaccttctcaaattcacactatattttgagaccaggagaaggctccttgagaaattgccacactgtcttatcctagtctctggaaaaattcagtcctgtattataactgggcgtttctcataagtgctttttttttttctttttctttttataatactttaagttctgggatacatgtgcagaatgtgcaggtttgttacataggtatacacgtgccatggtggtttgctgcacccatcaacccatcatctacattaggtatttctcctaatgctaccccttccctagccccccacccctcgacaggccccagtgtttgatgttcccctccctgtgtccatgtgttctcattgttcaactcccacttatgagtgagagcatgcggtgtttggttttctgttcctgtgttagtttgctgagaatgatggtttttggcttcatccacgtccctgcaaaggacatgaactcatcctttttatggctgcatagtattccatggtgtatatgtgccacatttgctttatccagtctatcattgatgagcacttgggttggctccaagtctttgctattatgactagtactgcgataaacatgtgtatatgtgtctttatattagaatgatttataatcctttaggtctatacccagtaataggattcctgggtcaaatggtatttctggttctagatccttgaggaattgccacactgtcttccacaacggttgaactaatttacactcccaccaacagtgtaaaagcattcctatttctccacatcctctccaacatctgttgtttcctgactctttaatgacggccattctaactggcgtgaaatggtatctcattgtggttttgatttgcatttctctaatgaccaccgatgatgagctttttctcatatgtttgctggccccataaatgtcttcttttgagaaatgtctgttcatatcctttgcccactttttgatggggttgtttgcttttttcttgtaaatttgtttgagttcctggtagattctggatattagccctttgtcagatggatagactgcaaaaattttctcccattctgtaggttgcctgttcactctgatggtagtttcttttgccatgcagaagctgtttagtttaattagatccaatttgtcagttttggcttttgttgccattgcttttggtgttttagtcatgaagtctttgcccatgcctatgtcctgaattgtattgccctggttttcttctaggatttttatggttttaggtcttacatttaagtctttaatccatcttgaattaatttttgtataaggtgtaaggaaagggtccagtttcagttttctgcgtatggctagccagttttcccaacgttatttattaaatagggaatcctttccccattgcttgtttttgtcaggtttgtcaaagatcagacggttgtagatgtgtggtgttatttctgaggcctctgttctgttccattggtctgtatatctgctttgataccagtaccgtgctgttttggttactgtagccttgtagtacagtttgaagtcaggtagcgtgatgctttgttctttttgcttaggattgtcttggctatatgggctcttttttggttctatatgaaatttaaagtagttttttcatagacatctatagaactctccaccccaaatcaacagaatatacattcttctcagtatcgcatcacacttattctaaaatgaccacataattagaagtaaaacactcctcagcaaatgcaaaaaacagaaatcctaacagtctgtcagaccccagtgcaatcaaattagaactcaggattaagaaacttactgaaaaccacacaactgcatggaaactgaacaacctgctcctgaatgactactgggtaaataacaaaattaaggcagaaataaataggttctttgaaagaaatgagaacaaagacacaatataccagaatctctgggacacagctaaagcagtgtttagagggaaattgatagcactaaatgcccacaggagaaagcgggacagatctaaaatcgacaccctaatatcacaattaaaagaactagagaaacaagagcaaacaaattcaaaagctagcagaagacaagaaataactaagatcagagcagaactgaaggagatagagacacaaaaaacccttcaaaaaatcaatgaatccaggagctgtttttttttttttgaaaagattaatgaaatagactgctagccaaactaataaaggagaaaagagagaagaatcaaatagacaatagacacaataaaaagtgataaaggggatatcaccactgatcccacagaaatacaaactaccatcagagaatactataaacacctctacgcaaataaactagaaaattgggaagaaatggatacattcctgaacgcatacaccctcccaagactaaaccaggaagaagttgaatccctgaatagaccaatagcaagttctgaaattgaggcagtaattagtagcctaccaaccaaaaaaagcccaggaccagacagattcacagccgaagtctaccagaggtacaaagaggagctagtaccattccttctgaaactattccaaacaatagaaaaagaaggactcctccctaactcattttatgaggccagcatcatcctgataccaaaacctggcagagacacaacataaaggaaattcaggccaatatccctgatgaacattgatgtgaaaatcctcaataaaatactggcagcacatcaaaaagcttacctgccatgatcaagttggcttcatccctgggatgcaaggctggttcaacatacgcaaatcaataaacgtaatcaatcacatacacagaaccaatgacaaaaaccacatgattatctcaatagatgcagaaaaggccttcgataaaattcaacaccccttcaatgctaagaactctcaataaactaggtattgatgaaacatatctcaaaataataagagttatttatgacaaacccacagccagtatcatactgaatgggcaaaagctggaagcattccctttgaaaaccgccacaaggatgacttctctcaccactcctattcaacatactattggaagttctggccagggcaagcaggcaagacaaataaataaagggtattcaaataggaagagaggaagtcaaattgtctctgtttgcagatgacatgattgtatatttagaaaaccccatcatctcagcccaaaatctccttaagctgataagcaacttcagcaaagtctcaggatacaaaatcaatgtgcaaaaatcacaagcattcctatacaccaataatagacaaacagccaaatcatgagtgaactcccattcacaagtgctacaaagaataaaatacctaggaatccaacttacaagggatgtgaaggacctcttcaaggagaactacagaccactgctcaaggtaataagagaggacacaaacaaatggaaaaacattccatgctcatggataggaagaatcaatattatgaaaatggccatactgcccaaaacaatttatagattcaatgccatccccatcaagctaccactgacttccttcacagaattagaaaaaaactactttaaatttcatatggaactgaaaaaagagccggtatagccaagacgatcctaagcaaaaagaaaaaagctggaagcatcatgctacctgacttcaaactatactacaaggctacagtaacaaaaacagcatggtatagtataagtgcttttttttaaaagacaaagtaaagtaatttttttgttgttggggtaaaacaaaagctctgcataaagagcagggatgttgtaacatacactgaccaaaggtgggaaacctacagttggagcagaagctgaatgtcacattatcagctccgaacttataatggtctaaaagtactaggttaatgttggaaagatggtgccatttaaagatatcttaaattcaatatttaattattttaatttgacattatcctagagttgaatggtgtttacttaccatgtgctgttcattagaaaatctagatcctacactgcctttgcgcaaggtagttgctctaataataccaacctgtccagttttggtgggagaaataatgttactgttaagttgcacttagtggttattatgtgtaatactggtattccaaagagagaaggaaaatgtttgctacacagctgtgttcttaggttcagaaaaccacaggagtgggacaggagaaccttcaggattcaggtccgattgttgtgatggccgcaggagggagactgtgaatttgaaactgcatccattgaaaagaaaatccctccacactttaataattctctccgtgccccatggcagcaagtgcttataggccttgctactcaaagcttaataaaaaggcagacctgctgaatcataatctgcatcttaacaatatccccagatattgtctgcactttctttttttttttctttctttctttcttcttttttttgagacagagtctcgctcagtcacccaggctggagtgcagtggcgcgatctcggctcactgcaagctctgactcccgggttcacgccattctcctgccttagactcccgagtagctggcactacaggcgcccgccactacgcccggctaatttttttgtatttttagtagagatggggtttcaccgtgttagccaggatggtctcgatctcctgacctcgtgatccgccgcccatctgggcctcccaaagtgctgggattacaggcgtgagccactgcgcccggccaattgtctgcactttcaagtctaagaagcactgtcccaggaggaaaatctttttagtctgaggcttctctcttgcactctcctttttaaaaatatgctgctccttctccacctttccctcttcttccgccttttctgtctgcctttactacctcccctgaacattcaacttgtagaagagttcccctttctctgaattgcattcttcacttcattcattcttttctctctctgtctatggtttcctattttttgtcggttttccctcacctcacctccttgttattttttgccattgttcacatatcactgccctttcagacccatatctagcttctgacccatccactaatccattgccactaatttattcagcatgcccatgccatttattaatgtaaatatttgcacatacttgttctatcatgcccatttttctaccttttaaattgtatatacacagacacatgtgaatgacatatttcactatttaaaaggtagaaaaatgtatatcgtggtacaaagtgatacattgagtatctgtgcccagtttcaagatgagaataagtgagaggtgaagcctcatgagtcacgtcacctacacgctactgtctttcattccatctgcagtactgccacacatttggttagttctccaattgctgtcacattgacatcgagttggatctgaacagcgtacctggggagacgaagtattggtatctttgcttaaatggagtgatttactgagagacaaagtgacatctttaagatcagatagcaagttactgcaccaggagcgcgacctttcctgttttgttgttttccctctgtcaaatgccttctggtctccatgaatctctccttccacatatgataaaaatgcaaagtctcataagcattcacagctcaggaagcctcaggctagttggggagaaaagactgggaggtttccggaggaatgaagtcctctgagcagagaggttaattcatcttgctgtaaaacaaaatagaaaataagttcccctcaataagtgaacgtaatacaaagacaaatgtggttggtgccagaggccaggaagaagttcttgtgagaataggtgcagagaagagctaggccctggctgagtttaaaccttgacttagtcactgtgggactctgggtgagttactccatggattgatggctgggtcatggagataatagtacctaattcatacaggtactgtgagaagtaaatggaatatttcacgttaagtgtttaacggtgcgtttaaatgctaggtgctattattattaatttttaaattaactttgccatgttttgtgtcttcccctctctgtgcttcctttctttagtatgagccgcacagcctacacggtgggagccctgcttctcctcttggggaccctgctgccggctgctgaagggaaaaagaaagggtcccaaggtgccatccccccgccagacaaggcccagcacaatgactcagagcagactcagtcgccccagcagcctggctccaggaaccgggggcggggccaagggcggggcactgccatgcccggggaggaggtgctggagtccagccaagaggccctgcatgtgacggagcgcaaatacctgaagcgagactggtgcaaaacccagccgcttaagcagaccatccacgaggaaggctgcaacagtcgcaccatcatcaaccgcttctgttacggccagtgcaactctttctacatccccaggcacatccggaaggaggaaggttcctttcagtcctgctccttctgcaagcccaagaaattcactaccatgatggtcacactcaactgccctgaactacagccacctaccaagaagaagagagtcacacgtgtgaagcagtgtcgttgcatatccatcgatttggattaagccaaatccaggtgcacccagcatgtcctaggaatgcagccccaggaagtcccagacctaaaacaaccagattcttacttggcttaaacctagaggccagaagaacccccagctgcctcctggcaggagcctgcttgtgcgtagttcgtgtgcatgagtgtggatgggtgcctgtgggtgtttttagacaccagagaaaacacagtctctgctagagagcactccctattttgtaaacatatctgctttaatggggatgtaccagaaacccacctcaccccggctcacatctaaaggggcggggccgtggtctggttctgactttgtgtttttgtgccctcctggggaccagaatctcctttcggaatgaatgttcatggaagaggctcctctgagggcaagagacctgttttagtgctgcattcgacatggaaaagtccttttaacctgtgcttgcatcctcctttcctcctcctcctcacaatccatctcttcttaagttgatagtgactatgtcagtctaatctcttgtttgccaaggttcctaaattaattcacttaaccatgatgcaaatgtttttcattttgtgaagaccctccagactctgggagaggctggtgtgggcaaggacaagcaggatagtggagtgagaaagggagggtggagggtgaggccaaatcaggtccagcaaaagtcagtagggacattgcagaagcttgaaaggccaataccagaacacaggctgatgcttctgagaaagtcttttcctagtatttaacagaacccaagtgaacagaggagaaatgagattgccagaaagtgattaactttggccgttgcaatctgctcaaacctaacaccaaactgaaaacataaatactgaccactcctatgttcggacccaagcaagttagctaaaccaaaccaactcctctgctttgtccctcaggtggaaaagagaggtagtttagaactctctgcataggggtgggaattaatcaaaaaccgcagaggctgaaattcctaatacctttcctttatcgtggttatagtcagctcatttccattccactatttcccataatgcttctgagagccactaacttgattgataaagatcctgcctctgctgagtgtacctgacagtagtctaagatgagagagtttagggactactctgttttagcaagagatattttgggggtctttttgttttaactattgtcaggagattgggctaaagagaagacgacgagagtaaggaaataaagggaattgcctctggctagagagtagttaggtgttaatacctggtagagatgtaagggatatgacctccctttctttatgtgctcactgaggatctgaggggaccctgttaggagagcatagcatcatgatgtattagctgttcatctgctactggttggatggacataactattgtaactattcagtatttactggtaggcactgtcctctgattaaacttggcctactggcaatggctacttaggattgatctaagggccaaagtgcagggtgggtgaactttattgtactttggatttggttaacctgttttcttcaagcctgaggttttatatacaaactccctgaatactctttttgccttgtatcttctcagcctcctagccaagtcctatgtaatatggaaaacaaacactgcagacttgagattcagttgccgatcaaggctctggcattcagagaacccttgcaactcgagaagctgtttttatttcgtttttgttttgatccagtgctctcccatctaacaactaaacaggagccatttcaaggcgggagatattttaaacacccaaaatgttgggtctgattttcaaacttttaaactcactactgatgattctcacgctaggcgaatttgtccaaacacatagtgtgtgtgttttgtatacactgtatgaccccaccccaaatctttgtattgtccacattctccaacaataaagcacagagtggatttaattaagcacacaaatgctaaggcagaattttgagggtgggagagaagaaaagggaaagaagctgaaaatgtaaaaccacaccagggaggaaaaatgacattcagaaccagcaaacactgaatttctcttgttgttttaactctgccacaagaatgcaatttcgttaacggagatgacttaagttggcagcagtaatcttcttttaggagcttgtaccacagtcttgcacataagtgcagatttggctcaagtaaagagaatttcctcaacactaacttcactgggataatcagcagcgtaactaccctaaaagcatatcactagccaaagagggaaatatctgttcttcttactgtgcctatattaagactagtacaaatgtggtgtgtcttccaactttcattgaaaatgccatatctataccatattttattcgagtcactgatgatgtaatgatatattttttcattattatagtagaatatttttatggcaagatatttgtggtcttgatcatacctattaaaataatgccaaacaccaaatatgaattttatgatgtacactttgtgcttggcattaaaagaaaaaaacacacatcctggaagtctgtaagttgttttttgttactgtaggtcttcaaagttaagagtgtaagtgaaaaatctggaggagaggataatttccactgtgtggaatgtgaatagttaaatgaaaagttatggttatttaatgtaattattacttcaaatcctttggtcactgtgatttcaagcatgttttctttttctcctttatatgactttctctgagttgggcaaagaagaagctgacacaccgtatgttgttagagtcttttatctggtcaggggaaacaaaatcttgacccagctgaacatgtcttcctgagtcagtgcctgaatctttattttttaaattgaatgttccttaaaggttaacatttctaaagcaatattaagaaagactttaaatgttattttggaagacttacgatgcatgtatacaaacgaatagcagataatgatgactagttcacacataaagtccttttaaggagaaaatctaaaatgaaaagtggataaacagaacatttataagtgatcagttaatgcctaagagtgaaagtagttctattgacattcctcaagatatttaatatcaactgcattatgtattatgtctgcttaaatcatttaaaaacggcaaagaattatatagactatgaggtaccttgctgtgtaggaggatgaaaggggagttgatagtctcataaaactaatttggcttcaagtttcatgaatctgtaactagaatttaattttcaccccaataatgttctatatagcctttgctaaagagcaactaataaattaaacctattctttctgtg

SEQ ID NO: 37 - DNA, 555 bp, Homo sapiens
  source 1..555: mol_type = genomic DNA
atgagccgcacagcctacacggtgggagccctgcttctcctcttggggaccctgctgccggctgctgaagggaaaaagaaagggtcccaaggtgccatccccccgccagacaaggcccagcacaatgactcagagcagactcagtcgccccagcagcctggctccaggaaccgggggcggggccaagggcggggcactgccatgcccggggaggaggtgctggagtccagccaagaggccctgcatgtgacggagcgcaaatacctgaagcgagactggtgcaaaacccagccgcttaagcagaccatccacgaggaaggctgcaacagtcgcaccatcatcaaccgcttctgttacggccagtgcaactctttctacatccccaggcacatccggaaggaggaaggttcctttcagtcctgctccttctgcaagcccaagaaattcactaccatgatggtcacactcaactgccctgaactacagccacctaccaagaagaagagagtcacacgtgtgaagcagtgtcgttgcatatccatcgatttggattaa